SEQUENCE LISTING (87 sequences)

SEQ ID NO: 1 - protein, 5 aa, synthetic construct
  REGION 1..5: note = Description of Artificial Sequence: Synthetic peptide
SYWMH

SEQ ID NO: 2 - protein, 5 aa, synthetic construct
  REGION 1..5: note = Description of Artificial Sequence: Synthetic peptide
NYGLH

SEQ ID NO: 3 - protein, 17 aa, synthetic construct
  REGION 1..17: note = Description of Artificial Sequence: Synthetic peptide
EIDPSDSYKDYNQKFKD

SEQ ID NO: 4 - protein, 16 aa, synthetic construct
  REGION 1..16: note = Description of Artificial Sequence: Synthetic peptide
VIWSGGSTDYNAAFIS

SEQ ID NO: 5 - protein, 9 aa, synthetic construct
  REGION 1..9: note = Description of Artificial Sequence: Synthetic peptide
AITTTPFDF

SEQ ID NO: 6 - protein, 14 aa, synthetic construct
  REGION 1..14: note = Description of Artificial Sequence: Synthetic peptide
GGIYYANHYYAMDY

SEQ ID NO: 7 - protein, 11 aa, synthetic construct
  REGION 1..11: note = Description of Artificial Sequence: Synthetic peptide
RASQSISNNLH

SEQ ID NO: 8 - protein, 16 aa, synthetic construct
  REGION 1..16: note = Description of Artificial Sequence: Synthetic peptide
KSSQSLLNSGNQKNYM

SEQ ID NO: 9 - protein, 7 aa, synthetic construct
  REGION 1..7: note = Description of Artificial Sequence: Synthetic peptide
YASQSIS

SEQ ID NO: 10 - protein, 7 aa, synthetic construct
  REGION 1..7: note = Description of Artificial Sequence: Synthetic peptide
GASTRES

SEQ ID NO: 11 - protein, 9 aa, synthetic construct
  REGION 1..9: note = Description of Artificial Sequence: Synthetic peptide
QQSNTWPYT

SEQ ID NO: 12 - protein, 9 aa, synthetic construct
  REGION 1..9: note = Description of Artificial Sequence: Synthetic peptide
QNDHSYPLT

SEQ ID NO: 13 - protein, 243 aa, synthetic construct
  REGION 1..243: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - DNA, 729 bp, synthetic construct
  misc_feature 1..729: note = Description of Artificial Sequence: Synthetic polynucleotide
  source 1..729: mol_type = other DNA
caggtccaattggtacagagcggcgtcgaagtaaagaagcctggagccagcgttaaagtttcttgcaaggcttcaggatatactttcactaactactatatgtactgggtacggcaggctccagggcaagggttggagtggatgggagggatcaatccttctaacggcggcactaactttaacgaaaaatttaaaaatagggtgaccctcacaactgactcaagtacgactacagcatacatggaactcaaatctctccaattcgatgacacggctgtctattattgcgcgagaagagactatcgcttcgatatggggtttgattattgggggcaaggtactacggttaccgtcagctccgggggtggcggctccggcggcggtgggtcaggtggaggagggtctgacattcagatgacgcaatccccaagctctctgtccgcgtcagtgggcgaccgagttacaatcacatgccgcgcttctcaagatgtgtcaaccgctgtcgcctggtaccaacagaagcctgggaaggcccctaagcttctcatctactcagcttcttttctgtactcaggggtaccgtctagattctcaggatccggtagtgggacggacttcacattgaccataagttccttgcagcctgaggatttcgctacatattattgccaacagtacctttaccatcctgccacttttggccagggtactaaggtcgagatcaaacgg

SEQ ID NO: 15 - protein, 244 aa, synthetic construct
  REGION 1..244: note = Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAGGGGSGGGGSGGGGSEIVLTQSPATLSLSPGERATLSCRASKGVSTSGYSYLHWYQQKPGEAPRLLIYLASYLESGVPARFSGSGSGTDFTLTISSLEPEDFAVYYCQHSRDLPLTFGGGTKVEIK

SEQ ID NO: 16 - DNA, 732 bp, synthetic construct
  misc_feature 1..732: note = Description of Artificial Sequence: Synthetic polynucleotide
  source 1..732: mol_type = other DNA
gaagttcagttggtcgagtcaggaggaggcctggtgcaacccgggggctcactccggttgtcctgtgctgcttcaggatttacgttttctgactcatggatacattgggtgcgccaagccccgggcaaggggctggaatgggtggcctggatctctccgtatgggggttccacctactatgctgattcagtaaaaggacggttcactataagcgcggatacaagtaagaatactgcctatcttcaaatgaattctcttcgcgccgaggatacagcggtatattattgcgctagacgacattggccagggggctttgactattgggggcagggtactcttgtgaccgttagtgcgggaggtggtggcagcggtggaggcggctccgggggtggtggttcagaaattgtcctgactcaatcccctgccacattgagtttgagcccaggagagagagcaactctgtcatgccgggcgtcaaaaggtgtcagtacgtcaggctactcctatcttcattggtatcagcagaaaccgggagaagcgccgcgccttctcatatacctggctagttaccttgagagtggcgtcccggcccggtttagtgggagtgggtctgggactgattttacgctgacaatcagcagtcttgagccagaggacttcgcggtttactattgccaacattcacgcgatttgcccctcaccttcggcggtggaacgaaggttgaaataaaa

SEQ ID NO: 17 - protein, 6 aa, synthetic construct
  REGION 1..6: note = Description of Artificial Sequence: Synthetic 6xHis tag
HHHHHH

SEQ ID NO: 18 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 19 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 20 - DNA, 54 bp, synthetic construct
  misc_feature 1..54: note = Description of Artificial Sequence: Synthetic oligonucleotide
  source 1..54: mol_type = other DNA
atgggatggagctctatcatcctcttcttggtagcaacagctacaggtgtccac

SEQ ID NO: 21 - protein, 19 aa, synthetic construct
  REGION 1..19: note = Description of Artificial Sequence: Synthetic peptide
MGWSCIILFLVATATGVHS

SEQ ID NO: 22 - protein, 19 aa, synthetic construct
  REGION 1..19: note = Description of Artificial Sequence: Synthetic peptide
MDWIWRILFLVGAATGAHS

SEQ ID NO: 23 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 24 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 25 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 26 - protein, 95 aa, Homo sapiens
MCCTKSLLLAALMSVLLLHLCGESEASNFDCCLGYTDRILHPKFIVGFTRQLANEGCDINAIIFHTKKKLSVCANPKQTWVKYIVRLLSKKVKNM

SEQ ID NO: 27 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 28 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 29 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 30 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 31 - protein, 133 aa, Homo sapiens [sequence omitted]

SEQ ID NO: 32 - protein, 120 aa, Homo sapiens
MKVSEAALSLLVLILIITSASRSQPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 33 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 34 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 35 - protein, 952 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 36 - DNA, 354 bp, synthetic construct
  misc_feature 1..354: note = Description of Artificial Sequence: Synthetic polynucleotide
  source 1..354: mol_type = other DNA
caggtccaactgcagcagcctggggctgagcttgtgaagcctggggcttcattgaagctgtcctgcaagtcttccgggtacaccttcaccagctactggatgcactgggtgaggcagaggcctggacatggccttgagtggatcggagagattgatccttctgacagttataaagactacaatcagaagttcaaggacaaggccacattgactgtggacagatcctccaacacagcctacatgcacctcagcagcctgacatctgatgactctgcggtctattattgtgcaagagcgattacgacgaccccctttgacttctggggccaaggcaccactctcacagtctcctca

SEQ ID NO: 37 - DNA, 324 bp, synthetic construct
  misc_feature 1..324: note = Description of Artificial Sequence: Synthetic polynucleotide
  source 1..324: mol_type = other DNA
gatattgtgctaactcagtctccagccaccctgtctgtgactccaggagatagcgtcagtctttcctgcagggccagccagagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggagtgtatttctgtcaacagagtaacacctggccgtacacgttcggaggggggaccaagctggaaataaaacgg

SEQ ID NO: 38 - DNA, 369 bp, synthetic construct
  misc_feature 1..369: note = Description of Artificial Sequence: Synthetic polynucleotide
  source 1..369: mol_type = other DNA
caggtgcagctgaagcagtcaggacctggcctagtgcagccctcacagagcctgtccatcacctgcacagtctctggtttctcattaactaactatggtttacactgggttcgccagtctccaggaaagggcctggagtggctgggagtgatatggagtggtggaagcacagactataatgcagctttcatatccagactgagcatcagcaaggacaactccaagagccaagttttctttaaaatgaacagtctgcaggctgatgacacagccatatactactgtgccagaaaaggagggatctactatgctaaccattactatgctatggactactggggtcaaggaacctcagtcaccgtctcctca

SEQ ID NO: 39 - DNA, 342 bp, synthetic construct
  misc_feature 1..342: note = Description of Artificial Sequence: Synthetic polynucleotide
  source 1..342: mol_type = other DNA
gacattgtgatgacacagtctccatcctccctgagtgtgtcagcaggagagaaggtcactatgagctgcaagtccagtcagagtctgttaaacagtggaaatcaaaagaactatatggcctggtatcagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccgctcacgttcggtgctgggaccaagctggagctgaaacgg

SEQ ID NO: 40 - DNA, 48 bp, synthetic construct
  misc_feature 1..48: note = Description of Artificial Sequence: Synthetic oligonucleotide
  source 1..48: mol_type = other DNA
ctcgagcccaaatcttgtgacaaaactcacacatgcccaccgtgcccg

SEQ ID NO: 41 - protein, 16 aa, synthetic construct
  REGION 1..16: note = Description of Artificial Sequence: Synthetic peptide
LEPKSCDKTHTCPPCP

SEQ ID NO: 42 - DNA, 81 bp, synthetic construct
  misc_feature 1..81: note = Description of Artificial Sequence: Synthetic oligonucleotide
  source 1..81: mol_type = other DNA
ttttgggtgctggtggtggttggtggagtcctggcttgctatagcttgctagtaacagtggcctttattattttctgggtg

SEQ ID NO: 43 - protein, 27 aa, synthetic construct
  REGION 1..27: note = Description of Artificial Sequence: Synthetic peptide
FWVLVVVGGVLACYSLLVTVAFIIFWV

SEQ ID NO: 44 - DNA, 126 bp, synthetic construct
  misc_feature 1..126: note = Description of Artificial Sequence: Synthetic polynucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 45 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 46 - DNA, 123 bp, synthetic construct
  misc_feature 1..123: note = Description of Artificial Sequence: Synthetic polynucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 47 - protein, 41 aa, synthetic construct
  REGION 1..41: note = Description of Artificial Sequence: Synthetic polypeptide
RSKRSRLLHSDYMNMTPRRPGPTRKHYQPYAPPRDFAAYRS

SEQ ID NO: 48 - DNA, 339 bp, synthetic construct
  misc_feature 1..339: note = Description of Artificial Sequence: Synthetic polynucleotide
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 49 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 15 aa, synthetic construct
  REGION 1..15: note = Description of Artificial Sequence: Synthetic peptide
GGGGSGGGGSGGGGS

SEQ ID NO: 51 - protein, 5 aa, synthetic construct
  REGION 1..5: note = Description of Artificial Sequence: Synthetic peptide
GGGGS

SEQ ID NO: 52 - protein, 10 aa, synthetic construct
  REGION 1..10: note = Description of Artificial Sequence: Synthetic peptide
EQKLISEEDL

SEQ ID NO: 53 - protein, 51 aa, Homo sapiens
PAKPTTTPAPRPPTPAPTIASQPLSLRPEACRPAAGGAVHTRGLDFACDIY

SEQ ID NO: 54 - protein, 49 aa, Mus sp.
KVNSTTTKPVLRTPSPVHPTGTSQPQRPEDCRPRGSVKGTGLDFACDIY

SEQ ID NO: 55 - protein, 51 aa, Felis sp.
PVKPTTTPAPRPPTQAPITTSQRVSLRPGTCQPSAGSTVEASGLDLSCDIY

SEQ ID NO: 56 - protein, 21 aa, Homo sapiens
IYIWAPLAGTCGVLLLSLVIT

SEQ ID NO: 57 - protein, 21 aa, Mus sp.
IWAPLAGICVALLLSLIITLI

SEQ ID NO: 58 - protein, 21 aa, Rattus sp.
IWAPLAGICAVLLLSLVITLI

SEQ ID NO: 59 - DNA, 105 bp, synthetic construct
  misc_feature 1..105: note = Description of Artificial Sequence: Synthetic polynucleotide
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 60 - protein, 35 aa, synthetic construct
  REGION 1..35: note = Description of Artificial Sequence: Synthetic polypeptide
TKKKYSSSVHDPNGEYMFMRAVNTAKKSRLTDVTL

SEQ ID NO: 61 - DNA, 108 bp, synthetic construct
  misc_feature 1..108: note = Description of Artificial Sequence: Synthetic polynucleotide
  source 1..108: mol_type = other DNA
agggaccagaggctgccccccgatgcccacaagccccctgggggaggcagtttccggacccccatccaagaggagcaggccgacgcccactccaccctggccaagatc

SEQ ID NO: 62 - protein, 36 aa, synthetic construct
  REGION 1..36: note = Description of Artificial Sequence: Synthetic polypeptide
RDQRLPPDAHKPPGGGSFRTPIQEEQADAHSTLAKI

SEQ ID NO: 63 - protein, 220 aa, synthetic construct
  REGION 1..220: note = Description of Artificial Sequence: Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 64 - protein, 8 aa, synthetic construct
  REGION 1..8: note = Description of Artificial Sequence: Synthetic peptide
  VARIANT 2: note = Val or Ile
  VARIANT 4: note = Any amino acid
DXEXNPGP

SEQ ID NO: 65 - protein, 118 aa, synthetic construct
  REGION 1..118: note = Description of Artificial Sequence: Synthetic polypeptide
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 66 - protein, 123 aa, synthetic construct
  REGION 1..123: note = Description of Artificial Sequence: Synthetic polypeptide
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSS

SEQ ID NO: 67 - protein, 108 aa, synthetic construct
  REGION 1..108: note = Description of Artificial Sequence: Synthetic polypeptide
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 68 - protein, 114 aa, synthetic construct
  REGION 1..114: note = Description of Artificial Sequence: Synthetic polypeptide
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 69 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 70 - protein, 330 aa, Homo sapiens [sequence omitted]

SEQ ID NO: 71 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 72 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 73 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 74 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 76 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 77 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 576 bp, synthetic construct
  misc_feature 1..576: note = Description of Artificial Sequence: Synthetic polynucleotide
  source 1..576: mol_type = other DNA
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 79 - DNA, 70 bp, synthetic construct
  misc_feature 1..70: note = Description of Artificial Sequence: Synthetic oligonucleotide
  source 1..70: mol_type = other DNA
gcaaatgggcggtaggcgtgtacggtgggaggtttatataagcagagctcgtttagtgaaccgtcagatc

SEQ ID NO: 80 - DNA, 153 bp, synthetic construct
  misc_feature 1..153: note = Description of Artificial Sequence: Synthetic polynucleotide
  source 1..153: mol_type = other DNA
aactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatc

SEQ ID NO: 81 - DNA, 546 bp, synthetic construct
  misc_feature 1..546: note = Description of Artificial Sequence: Synthetic polynucleotide
  source 1..546: mol_type = other DNA
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 82 - DNA, 1138 bp, synthetic construct
  misc_feature 1..1138: note = Description of Artificial Sequence: Synthetic polynucleotide
  source 1..1138: mol_type = other DNA
atgggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttata

SEQ ID NO: 83 - protein, 379 aa, synthetic construct
  REGION 1..379: note = Description of Artificial Sequence: Synthetic polypeptide
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIY

SEQ ID NO: 84 - DNA, 57 bp, Thosea asigna virus
  source 1..57: mol_type = unassigned DNA
gccgagggcagaggaagtcttctaacatgcggtgacgtggaggagaatcccggccct

SEQ ID NO: 85 - protein, 19 aa, Thosea asigna virus
AEGRGSLLTCGDVEENPGP

SEQ ID NO: 86 - protein, 18 aa, synthetic construct
  REGION 1..18: note = Description of Artificial Sequence: Synthetic peptide
MGWSSIILFLVATATGVH

SEQ ID NO: 87 - DNA, 30 bp, synthetic construct
  misc_feature 1..30: note = Description of Artificial Sequence: Synthetic oligonucleotide
  source 1..30: mol_type = other DNA
gagcagaagctgatcagcgaggaggacctg

(19) United States
(12) Patent Application Publication
(10) Pub. No.: US 2025/0179194 A1
(43) Pub. Date: Jun. 5, 2025

(54) COMPOSITIONS AND METHODS FOR IMMUNOTHERAPY TARGETING FLT3, PD-1, AND/OR PD-L1

(71) Applicant: Cytoimmune Therapeutics, Inc., Wilmington, DE (US)

(72) Inventors: Jianhua Yu, Wilmington, DE (US); Michael Caligiuri, Wilmington, DE (US)

(73) Assignee: Cytoimmune Therapeutics, Inc., Wilmington, DE (US)

(21) Appl. No.: 18/909,750

(22) Filed: Oct. 8, 2024

Related U.S. Application Data
(63) Continuation of application No. 17/257,553, filed on Dec. 31, 2020, now abandoned.
(60) Provisional application No. 62/693,977, filed on Jul. 4, 2018.

(51) Int. Cl. C07K 16/28; A61K 38/00; A61K 39/00; A61K 40/11; A61K 40/15; A61K 40/31; A61K 40/42; A61P 35/02; C07K 14/705; C07K 14/725; C12N 5/0783; C12N 15/86

(52) U.S. Cl. CPC C07K 16/2863; A61K 40/11; A61K 40/15; A61K 40/31; A61K 40/421; A61K 40/4214; A61P 35/02; C07K 14/7051; C07K 14/70517; C07K 14/70521; C07K 14/70578; C07K 16/2818; C07K 16/2827; C12N 5/0646; C12N 15/86; A61K 38/00; A61K 2039/507; A61K 2239/31; A61K 2239/38; A61K 2239/48; C07K 2317/31; C07K 2317/565; C07K 2317/622; C07K 2317/76; C07K 2319/02; C07K 2319/03; C07K 2319/30; C07K 2319/33; C12N 2510/00; C12N 2740/15043; C12N 2740/15071

(57) ABSTRACT

CAR cells targeting FLT3 antigens in combination with a secreted anti-PD-1 and anti-PD-L1 antibodies or anti-PD-1-anti-PD-L1 bispecific antibodies are described as a new method of cancer treatment. It is proposed that these combination therapies are safe and effective in patients and can be used to treat human tumors and cancer.

### CROSS-REFERENCE TO RELATED APPLICATIONS

This application is a continuation of U.S. application Ser. No. 17/257,553, filed Dec. 31, 2020, which is a national stage entry under U.S.C. § 371 of International Application No. PCT/US2019/040654, filed Jul. 3, 2019, which in turn claims priority under 35 U.S.C. § 119(e) to U.S. Provisional Application No. 62/693,977, filed Jul. 4, 2018, the content of each of which is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 29, 2025, is named 113086-0168_SL.xml and is 115,332 bytes in size.

### TECHNICAL FIELD

The present disclosure relates generally to the field of human immunology, specifically immunotherapy.

### BACKGROUND

Acute myeloid leukemia (AML) is a common hematological cancer. It is estimated that there will be 21,450 new cases predicted in 2019, see cancer.org/cancer/acute-myeloid-leukemia/about/key-statistics.htm, last accessed on Jul. 2, 2019. Though the disease responds to the induction and consolidation chemotherapy, relapse inevitably occurs in a majority of patients. The treatment protocol for AML patients over 65 years of age is especially unsuccessful with over 75% of patients dying of their disease within 5 years; thus, treatment has not been optimized. Therefore, novel treatment methods for this disease should be a priority. This disclosure provides such novel methods, compositions for use therein and related advantages as well.

### SUMMARY

Despite the heterogeneity of AML in general, many AML types share common FLT3 expression, which is present in around 80% of patients, with high surface density expression in approximately 20% of AML patients. In addition, as high as 80% of AML patient blasts could may express the ligand for PD-1, called PD-L1, while PD-1 surface expression can be found on a majority of T-cells from AML patients.

Chimeric antigen receptor (CAR) therapy is one of the most successful cellular immune therapies for lymphoid malignancies. A CAR is a genetically engineered immune surface receptor containing the Fab portion of an antibody directed against tumor antigen(s) and linked to intracellular domains of activation molecules such as CD28 and CD3ζ. Preclinical models of CARs expressed on T cells and natural killer (NK) cells directed against AML-associated antigens show promising treatment effects. Since NK-cells, but not T-cells, do not cause graft-versus-host disease (GVHD), CAR NK cells can be an important alternative to CAR T cells. In a recent study on anti-CD19 CAR T-cells, 30% of B-ALL patients did not respond to the CAR T therapy because of the alternatively spliced CD19 isoforms and compromised anti-CD19 CAR epitope. T cells lack “natural killing”, i.e., they require a CAR trigger to kill the tumor cells. NK cells possess spontaneous cytotoxicity and therefore can kill certain tumor targets such as AML even in the absence of a CAR being triggered. Therefore, the intrinsic cytolytic mechanisms of NK-cells can provide a secondary defense against the escape of cancer from CAR therapy. Understanding how CAR functions in other immune cells such as NK-cells will be impactful and will help create alternative or complementary methods in designing the next generation of CAR T- and CAR NK-cell therapy.

Checkpoint inhibitors such anti-PD-1 Pembrolizumab (Keytruda) and anti-PD-L1 Atezolizunab (Tecentriq) and others are being actively investigated in many clinical trials. However, some toxicities have hindered the further development. For example, a recent phase III clinical trial of Pembrolizumab has been halted because of the general toxicity. This could relate to massive T cell activation and a systemic autoimmune effect. Local delivery of an anti-PD-1 antibody, or an anti-PD-L1 antibody might be preferable. Likewise, the treatment of AML with CARs directed against CD123 or CD33 have yet to demonstrate any clinical success. Without being bound by theory, Applicant hypothesize that a CAR targeting FLT3 expressed on AML blasts will have therapeutic benefits to FLT3(+) AML patients with a better safety profile.

Moreover, the FDA-approved anti-PD-1 and anti-PD-L1 monoclonal antibodies (mAbs) are given systemically and bind to all cells expressing either PD-1(+) or PD-L1. The systemic intravenous infusion of these mAbs involves infusion of large doses and have been associated with mild, moderate and severe adverse effects including fatigue, pyrexia (fever), chills, and “infusion reactions” each requiring medical attention. Infusion reactions are infrequent but may result a serious loss in blood pressure requiring fluid resuscitation along with medications. Additional well-documented toxicities that range from mild to severe that are associated with the administration of these mAbs include dermatologic, gastrointestinal, endocrine, liver and lung toxicities. See, e.g., Naidoo et al. (2015) Annals of Oncology, Volume 26, Issue 12, Pages 2375-2391. The CAR T or NK cell of this disclosure eliminates the need for multiple separate intravenous infusions of the anti-PD-1 or anti-PD-L1 mAbs by engineering the CAR T or NK cell to secrete the anti-PD-1 or anti-PD-L1 mAbs once the CAR T or NK cells are infused inside the body. Accordingly, the CAR T or NK cell of this disclosure eliminates the need for large pharmacologic loading doses of the anti-PD-1 and/or anti-PD-L1 mAbs to be infused into the blood stream by separate, repeated administration. Based on the pharmacology and toxicity data published to date, it is reasonable to assume that this invention will avoid all PD-1(+) T or NK cells from becoming immediately saturated with anti-PD-1 mAb, very likely reducing some or all of the adverse events noted above. In addition, (1) the release of the antibody would only come from the CAR T or NK cells as they expand, so release of the anti-PD-1 or anti-PD-L1 mAbs into the bloodstream would be gradual; (2) given that the CAR homes the CAR T or NK cell to the tumor, the release of the anti-PD-1 or anti-PD-L1 mAbs would be more confined to the tumor microenvironment versus multiple massive intravenous systemic administrations as is currently FDA approved, and would therefore localize it's anti-tumor effect more to the relevant T or NK cells in the tumor microenvironment. Additionally, this approach would result in huge cost savings (approximately six figure cost savings) to the patient as there would be no need for anti-PD-1 or anti-PD-L1 mAb infusions.

Unexpectedly, it has recently been reported that human natural killer cells from cancer patients express PD-L1 and that anti-PD-L1 antibody bound to NK cells was unexpectedly found to increase the NK cell killing of the tumor cell. Thus, this disclosure, whereby infected NK cells express both a CAR and a secreted anti-PD-L1 could significantly enhance NK killing of the tumor cell locally at the tumor microenvironment.

To that end, disclosed herein is/are one or more vectors or isolated polynucleotides comprising, or alternatively consisting essentially of, or further consisting of, a polynucleotide encoding a nucleic acid sequence of a chimeric antigen receptor (CAR) comprising: (a) an antigen binding domain of an FLT3 antibody; (b) a hinge domain; (c) a transmembrane domain; (d) and an intracellular domain; and a polynucleotide encoding a nucleic acid sequence of an antibody comprising an antigen binding domain that recognizes and binds PD-1 and/or PD-L1. In some embodiments, a contiguous polynucleotide or a single vector further comprises, or further consists essentially of, or yet further consists, of both the polynucleotide encoding the nucleic acid sequence of a CAR comprising: (a) an antigen binding domain of an FLT3 antibody; (b) a hinge domain; (c) a transmembrane domain; (d) and an intracellular domain; and the polynucleotide encoding the nucleic acid sequence of an antibody comprising an antigen binding domain that recognizes and binds PD-1 and/or PD-L1. In other embodiments, provided herein is an isolated nucleic acid or a vector comprising, or alternatively consisting essentially of, or yet further consisting of:
a. a polynucleotide encoding a chimeric antigen receptor (CAR) comprising, or alternatively consisting essentially of, or yet further consisting of: (a) an antigen binding domain of an FLT3 antibody; (b) a hinge domain; (c) a transmembrane domain; (d) and an intracellular domain; and
b. a polynucleotide encoding an antibody or antigen binding fragment thereof comprising, or alternatively consisting essentially of, or yet further consisting of an antigen binding domain that recognizes and binds PD-1 and/or PD-L1.

In further embodiments, the vector may be polycistronic, optionally bicistronic, and/or each of the polynucleotides may be operatively linked to regulatory polynucleotide sequences, such as enhancer elements and/or promoter elements.

An example of a contiguous polynucleotide of this disclosure is shown in FIG. 1. In some embodiments, the disclosure provides that each of the polynucleotides encoding the nucleic acid sequence of a CAR as disclosed above and within, and in one aspect, the CAR comprising: (1) (a) an antigen binding domain of an FLT3 antibody; (b) a hinge domain; (c) a transmembrane domain; (d) and an intracellular domain; and (2) the polynucleotide encoding the nucleic acid sequence of an antibody or antigen binding fragment thereof comprising an antigen binding domain that recognizes and binds PD-1 and/or PD-L1, the polynucleotides of (1) and (2) are comprised on separate independent polynucleotides or within separate vectors.

In any of the above embodiments, the vector or vectors is/are a plasmid or a viral vector, optionally selected from the group of a retroviral vector, a lentiviral vector, an adenoviral vector, and an adeno-associated viral vector.

In any of the above embodiments, the polynucleotides and/or vector or vectors may optionally comprise, or alternatively consist essentially of, or further consist, of a detectable label and/or a polynucleotide conferring antibiotic resistance and/or regulatory elements for the transcription and translation for the CAR and the antigen binding domain that recognizes and binds PD-1 and/or PD-L1. In a further aspect, the therapeutic methods are combined with diagnostic methods that identify the subject or patient suitable for therapy by analyzing a suitable sample isolated from the patient of subject for expression of FLT3 and/or PD-1 and/or PD-L1, and determining that the patient or subject expresses one, two or three of FLT3, PD-1 and/or PD-L1, is suitable for the therapy. In a further aspect, the therapy is then administered to the subject or patient. Suitable samples include those that comprise cancer and/or tumor cells.

The isolated nucleic acid or vector disclosed above encoding the CAR can comprise, or alternatively consist essentially of, or yet further consist of any CAR disclosed herein. In one aspect, the isolated nucleic acid or the vector of this disclosure encoding the CAR further comprises, or alternatively consists essentially of, or yet further consists of a signaling domain. In another aspect, the isolated nucleic acid or the vector encoding the CAR further comprises an inducible or a constitutively active element. In one embodiment, the inducible or the constitutively active element controls the expression of a polynucleotide encoding an immunoregulatory molecule or a cytokine. The immunoregulatory molecule or cytokine can comprise, or alternatively consist essentially of, or yet further consist of one or more of B7.1, CCL19, CCL21, CD40L, CD137L, GITRL, GM-CSF, IL-12, IL-2, low-toxicity IL-2, IL-15, IL-18, IL-21, LEC, and/or OX40L. In another aspect, the immunoregulatory molecule or cytokine can comprise, or alternatively consist essentially of, or yet further consist of IL-12 and/or GM-CSF; and/or IL-12 and/or one or more of IL-2 and low-toxicity IL-2; and/or IL-12 and/or IL-15; and/or IL-12 and/or IL-21; IL-12 and/or B7.1; and/or IL-12 and/or OX40L; and/or IL-12 and/or CD40L; and/or IL-12 and/or GITRL; and/or IL-12 and/or IL-18; and/or one or more of IL-2 and low-toxicity IL-2 and one or more of CCL19, CCL21, and LEC; and/or IL-15 and one or more of CCL19, CCL21, and LEC; and/or IL-21 and one or more of CCL19, CCL21, and LEC; and/or GM-CSF and one or more of CCL19, CCL21, and LEC; and/or OX40L and one or more of CCL19, CCL21, and LEC; and/or CD137L and one or more of CCL19, CCL21, and LEC; and/or comprises B7.1 and one or more of CCL19, CCL21, and LEC; and/or CD40L and one or more of CCL19, CCL21, and LEC; and/or GITRL and one or more of CCL19, CCL21, and LEC.

In any of the above embodiments, each of the polynucleotides may be operatively linked to a regulatory polynucleotide, optionally a promoter and/or enhancer. In some embodiments, the polynucleotide encoding an antibody or antigen binding fragment thereof comprising an antigen binding domain that recognizes and binds PD-1 and/or PD-L1 is operatively linked to a promoter and/or enhancer which allows for low, moderate or high expression, or yet further overexpression of the antibody or antigen binding fragment thereof.

In any of the above embodiments, the polynucleotide encoding the CAR may comprise, or alternatively consist essentially of, or further consist of a polynucleotide encoding: (a) an antigen binding domain of a FLT3 antibody; (b) a CD8a hinge domain; (c) a CD8a transmembrane domain; (d) a CD28 costimulatory signaling region and/or a 4-1BB costimulatory signaling region; and (e) a CD3 zeta (ξ) signaling domain. Non-limiting examples of the FLT3 antibody comprise, or alternatively consist essentially of, or further consist of a heavy chain variable region comprising: a CDHR1 having the amino acid sequence (SYWMH (SEQ ID NO: 1)) or (NYGLH (SEQ ID NO: 2)) or an equivalent of each thereof, a CDHR2 having the amino acid sequence (EIDPSDSYKDYNQKFKD (SEQ ID NO: 3)) or (VIWSGGSTDYNAAFIS (SEQ ID NO: 4)) or an equivalent of each thereof, and a CDHR3 having the amino acid sequence (AITTTPFDF (SEQ ID NO: 5)) or (GGIYYANHYYAMDY (SEQ ID NO: 6)) or an equivalent of each thereof, and/or a light chain variable region comprising: a CDLR1 having the amino acid sequence (RASQSISNNLH (SEQ ID NO: 7)) or (KSSQSLLNSGNQKNYM (SEQ ID NO: 8)) or an equivalent of each thereof, a CDLR2 having the amino acid sequence (YASQSIS (SEQ ID NO: 9)) or (GASTRES (SEQ ID NO: 10)) or an equivalent of each thereof, and a CDLR3 having the amino acid sequence (QQSNTWPYT (SEQ ID NO: 11)) or (QNDHSYPLT (SEQ ID NO: 12)) or an equivalent of each thereof.

Non-limiting examples of the antibody comprising an antigen binding domain that recognizes and binds PD-1 and/or PD-L1 or antigen binding fragment thereof include a PD-1 antagonist or agonist and/or a PD-L1 antagonist or agonist. In certain embodiments, the antibody or antigen binding fragment thereof comprises, or alternatively consists essentially thereof, or further consists of the relevant CDR regions of an antibody to PD-1 and/or an antibody to PD-L1, or an equivalent of each thereof. In some embodiments, the antibody or antigen binding fragment thereof comprises, or alternatively consists essentially thereof, or further consists of the heavy chain and/or light chain variable region of an antibody to PD-1 and/or PD-L1, and/or an equivalent of each thereof. In some embodiments, the antibody or antigen binding fragment thereof comprises, or consists essentially of, or yet further consists of a single chain variable fragment (scFv) comprising an antigen binding domain of a PD-1 antibody and/or a single chain variable fragment (scFv) comprising an antigen binding domain of a PD-L1 antibody, and/or an equivalent each thereof. In some embodiments, the antibody comprising an antigen binding domain that recognizes and binds PD-1 and/or PD-L1 or antigen binding fragment thereof comprises, or consists essentially of, or yet further consists of a single chain variable fragment (scFv) comprising an antigen binding domain of a PD-L1 antibody.

In some embodiments, the antibody or antigen binding fragment is a bispecific antibody. Non-limiting examples of the bispecific antibody include the relevant CDR regions of an antibody to PD-1 and an antibody to PD-L1, or an equivalent of each thereof, and, optionally a linker. Additional non-limiting examples include the relevant CDR regions of an antibody to PD-1 and an antibody to PD-L1, or an equivalent of each thereof and, optionally a linker. An additional example includes the heavy chain and/or light chain variable region of an antibody to PD-1 and/or PD-L1, and an equivalent of each thereof and, optionally a linker. A yet further example includes a single chain variable fragment (scFv) comprising, or alternatively consisting essentially of, or yet further consisting of, an antigen binding domain of a PD-1 antibody and a single chain variable fragment (scFv) comprising, or alternatively consisting essentially of, or yet further consisting of, an antigen binding domain of a PD-L1 antibody, and/or an equivalent each thereof and, optionally a linker.

Also provided herein is an isolated cell comprising, or alternatively consisting essentially of, or consisting of any one or more of the antibodies, the vectors and/or isolated polynucleotides of any one of the above embodiments, alone or in combination with each other. The cell can be a prokaryotic or a eukaryotic cell, and is optionally selected from an animal cell, a mammalian cell, a bovine cell, a feline cell, a canine cell, a murine cell, an equine cell or a human cell. In some embodiments, the eukaryotic cell is an immune cell, optionally a T-cell, a B-cell, a NK-cell, a dendritic cell, a myeloid cell, a monocyte, or a macrophage. In further embodiments, the immune cell is a T-cell, which may be optionally modified to suppress endogenous TCR expression, using any appropriate system, e.g., a CRISPR system. In any of the above embodiments relating to an isolated cell, the isolated cell expresses the CAR on the cell surface and secretes the antibody comprising an antigen binding domain that recognizes and binds PD-1 and/or PD-L1 or antigen binding fragment thereof, optionally the bispecific antibody.

Still further aspects relate to a composition comprising any one or more of the vectors and/or isolated nucleic acids and/or isolated cells as disclosed herein and, optionally, a carrier that is optionally a pharmaceutically acceptable carrier. Also provided herein is a composition comprising, or alternatively consisting essentially of, or yet further consisting of the isolated nucleic acid or vector, the antibody, the antigen binding fragment, the polypeptide, the isolated cell and/or the population of cells disclosed herein, and, optionally, a pharmaceutically acceptable carrier. In further embodiments, the composition comprises, or alternatively consisting essentially of, or yet further consisting of, an effective amount of a FLT3 inhibitor. In some embodiments, the effective amount is an amount effective to increase FLT3 surface expression on cancer or tumor cells.

This disclosure also provides an isolated complex comprising any of the isolated cells expressing the CAR bound to: (i) a cell expressing FLT3 and/or PD-1 and/or PD-L1 and/or a fragment thereof and/or, (ii) FLT3 and/or PD-1 and/or PD-L1 and/or a fragment thereof.

Methods to produce a CAR expressing cell is also disclosed. The methods comprise transducing an isolated cell with a polynucleotide or a vector as disclosed herein. In some embodiments, the isolated cells are selected from a group consisting of T-cells, B-cells, NK-cells, dendritic cells, myeloid cells, monocytes, or macrophages. In some embodiments, the isolated cells are selected from a group consisting of T-cells, B-cells, NK-cells, dendritic cells, myeloid cells, monocytes, or macrophages. In further embodiments, the isolated cells are T-cells, which are optionally modified to suppress endogenous TCR expression. In further embodiments, the isolated cells are NK cells. The cells can be isolated from any appropriate species, e.g., mammalian such as a human cell.

The CAR expressing cells are useful diagnostically and therapeutically. In one aspect, the cells are useful in a method of inhibiting the growth of a cancer cell or tumor each expressing FLT3, optionally wherein the cell is a FLT3 acute myeloid leukemia (AML) cell. This disclosure also relates to methods of inhibiting the growth of a cancer or tumor expressing FLT3, optionally acute myeloid leukemia (AML) in a subject, comprising, or alternatively consisting essentially of, or yet further consisting of contacting the cancer or tumor with the isolated cell of or the composition of this disclosure. In one aspect, the method of inhibiting the growth of a cancer or tumor expressing FLT3 in a subject, optionally AML, comprises, or alternatively consists essentially of, or yet further consists of measuring expression of PD-1 and/or PD-L1 in the subject and administering the isolated cell, the antibody, the antigen binding fragment and/or the composition of this disclosure to a subject expressing PD-1 and/or PD-L1. Further disclosed herein are methods of inhibiting the growth of a cancer or tumor in a subject, optionally AML, comprising, or alternatively consisting essentially of, or yet further consisting of measuring expression of PD-1 and/or PD-L1 in the subject and administering the antibody, the antigen binding fragment and/or the composition to a subject expressing PD-1 and/or PD-L1. The methods may comprise, or alternatively consist essentially of, or yet further consist of contacting the cancer cell or tumor with any of the isolated cells or compositions disclosed herein above. The contacting may be in vitro or in vivo. In some embodiments, the contacting is in vivo and the isolated cells are autologous and/or allogeneic to a subject being treated. In further embodiments, the method further comprises, or consists essentially of, or yet further consists of, administering to the subject an effective amount of cytoreductive therapy, optionally comprising or selected from the group consisting of chemotherapy, cryotherapy, hyperthermia, targeted therapy, and/or radiation therapy. In some embodiments, the subject being treated is a human patient.

Further provided herein are antibodies comprising, or alternatively consisting essentially of, or yet further consisting of a single chain variable fragment sequence (scFv) comprising, or alternatively consisting essentially of, or yet further consisting of an amino acid sequence of (Q V Q L V Q S G V E V K K P G A S V K V S C K A S G Y T F T N Y Y M Y W V R Q A P G Q G L E W M G G I N P S N G G T N F N E K F K N R V T L T T D S S T T T A Y M E L K S L Q F D D T A V Y Y C A R R D Y R F D M G F D Y W G Q G T T V T V S S G G G G S G G G G S G G G G S D I Q M T Q S P S S L S A S V G D R V T I T C R A S Q D V S T A V A W Y Q Q K P G K A P K L L I Y S A S F L Y S G V P S R F S G S G S G T D F T L T I S S L Q P E D F A T Y Y C Q Q Y L Y H P A T F G Q G T K V E I K R (SEQ ID NO: 13)) or an equivalent thereof. In one aspect, the antibody comprising, or alternatively consisting essentially of, or yet further consisting of a single chain variable fragment sequence (scFv) is encoded by the nucleotide sequence comprising, or alternatively consisting essentially of, or yet further consisting of a nucleic acid sequence of:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
or
an equivalent thereof.

Also described herein are antibodies comprising, or alternatively consisting essentially of, or yet further consisting of a single chain variable fragment sequence (scFv) comprising, or alternatively consisting essentially of, or yet further consisting of an amino acid sequence of:

(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15))
or
an equivalent thereof.

In one aspect, the antibody comprises, or alternatively consists essentially of, or yet further consists of a single chain variable fragment sequence (scFv) encoded by the nucleotide sequence comprising, or alternatively consisting essentially of, or yet further consisting of a nucleic acid sequence of:

(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent thereof.

Also described herein is a bispecific antibody comprising, or alternatively consisting essentially of, or yet further consisting of a single chain variable fragment sequence (scFv) comprising, or alternatively consisting essentially of, or yet further consisting of an amino acid sequence of:

(QVQLVQSGVEVKKPGASVKVSCKASGYTFTNY
YMYWVRQAPGQGLEWMGGINPSNGGTNFNEKFKNRVTLTT
DSSTTTAYMELKSLQFDDTAVYYCARRDYRFDMGFDYWGQ
GTTVTVSSGGGGSGGGGSGGGGSDIQMTQSPSSLSASVGDR
VTITCRASQDVSTAVAWYQQKPGKAPKLLIYSASFLYSGVPS
RFSGSGSGTDFTLTISSLQPEDFATYYCQQYLYHPATFGQGT
KVEIKR (SEQ ID NO: 13))
and/or
(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15))
or
an equivalent of each thereof.

In one aspect, the bispecific antibody comprises, or alternatively consists essentially of, or yet further consists of a single chain variable fragment sequence (scFv) encoded by the nucleotide sequence comprising, or alternatively consisting essentially of, or yet further consisting of a nucleic acid sequence of:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
and/or
(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent thereof.

The antibody can be an IgA, an IgD, an IgE, an IgG or an IgM antibody. In one particular aspect, the antibody comprises, or alternatively consists essentially of, or yet further consists of a constant region. The constant region may comprise, or alternatively consist essentially of, or yet further consist of an IgA, an IgD, an IgE, an IgG or an IgM constant region. In some embodiments, the constant region is an IgGI constant region or an Ig kappa constant region.

This disclosure also relates to antibodies that competes for binding with the antibodies described herein. The antibody of this disclosure may be a polyclonal, a monoclonal or a humanized antibody. Also provided herein are the antigen binding fragments of the antibodies of this disclosure. The antigen binding fragment may be selected from the group consisting of Fab, F(ab′)2, Fab′, scFv, and Fv. In one aspect, the antigen binding fragment may comprise, or alternatively consist essentially of, or yet further consist of an amino acid sequence of:

(QVQLVQSGVEVKKPGASVKVSCKASGYTFTNYYM
YWVRQAPGQGLEWMGGINPSNGGTNFNEKFKNRVTLTTDS
STTTAYMELKSLQFDDTAVYYCARRDYRFDMGFDYWGQGT
TVTVSSGGGGSGGGGSGGGGSDIQMTQSPSSLSASVGDRVTI
TCRASQDVSTAVAWYQQKPGKAPKLLIYSASFLYSGVPSRF
SGSGSGTDFTLTISSLQPEDFATYYCQQYLYHPATFGQGTKV
EIKR (SEQ ID NO: 13))
or
an equivalent of each thereof.

The antigen binding fragment can be encoded by the nucleotide sequence comprising, or alternatively consisting essentially of, or yet further consisting of a nucleic acid sequence of:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
or
an equivalent of each thereof.

In another aspect, the antigen binding fragment may comprise, or alternatively consist essentially of, or yet further consist of an amino acid sequence of:

(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15))
or
an equivalent of each thereof.

The antigen binding fragment can be encoded by the nucleotide sequence comprising, or alternatively consisting essentially of, or yet further consisting of:

(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
AGCCCAGGAGAGAGAGCAACTCTGTCATGCCGGGCGTCAAAAGGTGTCA
GGTGGTTCAGAAATTGTCCTGACTCAATCCCCTGCCACATTGAGTTTGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent of each thereof.

Also described herein are polypeptides comprising, or alternatively consisting essentially of, or yet further consisting of an amino acid sequence of any one of:

(QVQLV
QSGVEVKKPGASVKVSCKASGYTFTNYYMYWVRQAPGQGL
EWMGGINPSNGGTNFNEKFKNRVTLTTDSSTTTAYMELKSL
QFDDTAVYYCARRDYRFDMGFDYWGQGTTVTVSSGGGGSG
GGGSGGGGSDIQMTQSPSSLSASVGDRVTITCRASQDVSTAV
AWYQQKPGKAPKLLIYSASFLYSGVPSRFSGSGSGTDFTLTI
SSLQPEDFATYYCQQYLYHPATFGQGTKVEIKR (SEQ ID
NO: 13))
or
(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15))
or
an equivalent of each thereof.

This disclosure further relates to isolated nucleic acids comprising, or alternatively consisting essentially of, or yet further consisting of a nucleic acid sequence of:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
or
(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent of each thereof.

Further disclosed herein are methods of preparing the antibodies of this disclosure.

Also provided herein are the antigen binding fragments of the antibodies of this disclosure. Further described herein are polypeptides comprising, or alternatively consisting essentially of, or yet further consisting of an amino acid sequence of:

(QVQLVQSGVE
VKKPGASVKVSCKASGYTFTNYYMYWVRQAPGQGLEWMG
GINPSNGGTNFNEKFKNRVTLTTDSSTTTAYMELKSLQFDDT
AVYYCARRDYRFDMGFDYWGQGTTVTVSSGGGGSGGGG SG
GGGSDIQMTQSPSSLSASVGDRVTITCRASQDVSTAVAWYQ
QKPGKAPKLLIYSASFLYSGVPSRFSGSGSGTDFTLTISSLQP
EDFATYYCQQYLYHPATFGQGTKVEIKR (SEQ ID NO: 13))
or
(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15))
or
an equivalent of each thereof.

This disclosure further relates to isolated nucleic acids comprising, or alternatively consisting essentially of, or yet further consisting of a nucleic acid sequence of:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
or
(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent of each thereof.

Also disclosed herein are kits comprising, or alternatively consisting essentially of, or consisting of one or more of the above noted compositions and instructions for their use in the methods as disclosed herein.

### BRIEF DESCRIPTION OF THE DRAWINGS

FIG. 1 shows the design of bicistronic FLT3 CAR with secretory PD-1-PD-L1 bispecific antibody (biAb). FLT3 CAR was driven by EF1α promoter. PD-1-PD-L1 biAb was linked with CAR by T2A and led by a secretory signal peptide (SS). The whole cassette was flanked with long terminal repeats in a safe lentiviral construct.

FIGS. 2A-2C show enhanced cytotoxicity upon recognizing FLT3(+) AML cell lines. Cytotoxic activity of unmodified NK-92 cells, EV NK-92 cells, and FLT3 CAR NK-92 cells against: (FIG. 2A) FLT3(+) MOLM-13 (FLT3(+) EOL-1 or (FLT3(−) U937 cells; (FIG. 2B) FLT3(+) AML blasts from each of two patients using a ⁵¹Cr release assay. Target leukemic cells were labeled with ⁵¹Cr and co-cultured with NK-cells at the indicated effector/target ratios (E/T) in the wells of 96-well V-bottom plate at 37° C. for 4 hours. Supernatants were harvested and the release of ⁵¹Cr was measured on TopCount counter (Canberra Packard). (FIG. 2C) Cytotoxicity of primary FLT3 CAR NK-cells against normal hematopoietic stem cells (HSCs) defined as CD34(+) and dendritic cells defined as CD123(+) from bone marrow of healthy donors. Topro3(+) cells were considered dead.

FIGS. 3A-3C show FLT3 CAR T and FLT3 NK-cells suppress in vivo growth of human AML and prolong the survival of AML-bearing mice. NSG mice were injected with FLT3(+) MOLM-13 cells (FIG. 3A) or FLT3(+) AML patient blasts (FIG. 3B). A week later T-cells, T-cells with empty vector or FLT3 CAR T-cells were intravenously injected. (FIG. 3C) For FLT3(+) CAR NK-cells, NSG mice were injected with FLT3(+) MOLM-13 cells and NK, NK with empty vector or FLT3 CAR NK (shown here as “FLT3 CAR”, or PBS were infused. Kaplan-Meier curves were plotted. Δ=continuous CR.

FIGS. 4A-4D show FLT-3 CAR NK-cells and expanded primary NK-cells expressed high level of PD-1. (FIG. 4A) CAR NK-cells were in culture after transduction and stained for anti-PD-1 antibody. (FIG. 4B) Primary NK-cells (without CAR transduction) express PD1 in culture with IL-2, IL-15 and IL-21. The PD1 expression was not necessary for CAR transduction. (FIGS. 4C and 4D) AML cell lines K562 and Molm-13 expressed anti-PD-1-anti-PD-L1 biAb. Data are from one representative run.

FIGS. 5A-5B show secretion levels of the anti-PD-1-anti-PD-L1 biAb protein by T-cells transduced with an FLT3 CAR-anti-PD-1-anti-PD-L1 biAb vector. The section levels were determined by ELISA using a 6×-his tag antibody (“6×-his tag” disclosed as SEQ ID NO: 17) 2 and 3 days post transduction. (FIG. 5A). Standard curves of ELISA. Data from two times are shown. (FIG. 5B). Secretion levels of anti-PD-1-anti-PD-L1 biAb from the FLT3 CAR-anti-PD-1-anti-PD-L1 biAb-transduced T-cells were detected by ELISA using shown the 6×-his tag (SEQ ID NO: 17). Repeated data (Red and blue) of two times are shown. Data were collected from values of OD 450 nm based on the standard curve shown in FIG. 5A.

FIGS. 6A-6B show secretion levels of T-cells transduced with FLT3 CAR-anti-PD-1, FLT3 CAR-anti-PD-L1, or FLT3 CAR-PD-1-PD-L1 biAb vectors, determined by ELISA using 6×-his tag antibody (“6×-his tag” disclosed as SEQ ID NO: 17) 5 days post transduction. (FIG. 6A) Standard curves of ELISA. Data from two times are shown. (FIG. 6B) Secretion levels of anti-PD-1, anti-PD-L1, or PD-1-PD-L1 biAb by T-cells transduced with a FLT3 CAR-anti-PD-1, FLT3 CAR-anti-PD-L1, or FLT3 CAR-anti-PD-1-anti-PD-L1 biAb vector, respectively. The secretion levels were determined by ELISA using shown the 6×-his tag antibody. Data from three different donors (24, 25, 26, 27) are shown.

FIG. 7 shows infection and purification of FLT3 CAR T-cells expressing anti-PD-L1, anti-PD-1, anti-PD-1-anti-PD-L1 biAb, or detected by flow cytometry after cells were stained with anti-Fab against CAR. Data of one representative donor are shown.

FIGS. 8A-8B report the results of four hour flow-based killing assays, which showed that purified FLT3 CAR-anti-PD-1 T-cells maintain cytotoxicity levels as purified FLT3 CAR T-cells. (FIG. 8A) Two FLT3(+) AML tumor cell lines and one (FLT3(−) AML tumor cell line were used as target cells. All tumor cells were pretreated by AM Fluorescent Dye. Cells were gated on AML target cells and cells positive for Sytox Blue (Y-axis) represent the capacity of tumor cells being killed. (FIG. 8B) Summary data of FIG. 8A with three E:T (effector:target) ratios.

FIG. 9 shows that secreted anti-PD-1 Ab from FLT3 anti-PD-1 CAR-T-cells increase viability of FLT3 CAR T-cells. Gray column shows the untreated healthy donors' primed T-cells. Black column shows FLT3 CAR T-cells (as negative control). Light green column shows FLT3 CAR T-cells cultured with supernatant containing secreted anti-PD-1 Ab (15 ng/ml). Light green column with hatched pattern shows FLT3 CAR T-cells pretreated with a supernatant containing 15 ng/ml secreted anti-PD-1 Ab incubated with 10 μg/ml PD-1 fusion protein for 30 mins. Dark green column shows FLT3 CAR T-cells cultured with anti-PD-1 Ab (BD Biosciences, as positive control). Blue columns show FLT3-anti-PD-1 CAR T-cells cultured with supernatant containing secreted anti-PD-1 Ab (15 ng/ml). Blue column with hatched pattern shows FLT3-anti-PD-1 CAR T-cells cultured with a supernatant containing secreted 15 ng/ml anti-PD-1 ab incubated with 10 μg/ml PD-1 fusion protein for 30 mins. Data are summarized from 7 donors. Cell proliferation was assessed as MTT assays.

FIG. 10 shows the quantification of surface FLT3 expression in MOLM-13, U937, THP-1, MV4-11, and EOL-1 AML cell lines treated with vehicle control or the following FLT3 inhibitors for 48 hours: 10 μM Midostaurin, 10 μM FF-10101, 10 μM Quizartinib (AC220), or 10 μM Dovitinib (TKI-258). The data show that in most instances, FLT3 surface density expression was increased following treatment with a FLT3 inhibitor.

FIG. 11 depicts the quantification of FLT3 surface density expression by flow cytometry on AML blasts before and after being treated with Midostaurin for 48 hours. The figure shows that FLT3 surface density expression on the AML blasts was upregulated after the treatment.

FIG. 12 depicts the proposed mechanism of FLT3 anti-PD-1-anti-PD-L1 CAR NK and FLT3 anti-PD-1-anti-PD-L1 CAR T cells interacting with a FLT3(+) AML blast and secreting anti-PD-1-anti-PD-L1 biAb that blocks the PD1-PD-L1 interaction between the PD-1(+) T cell and/or NK cell, and the PD-L1(+) leukemic blast.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Green and Sambrook eds. (2012) Molecular Cloning: A Laboratory Manual, 4^{th} edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modem Applications; Immobilized Cells and Enzymes (IRL Press (1986)); Grandi ed. (2007) In Vitro Transcription and Translation Protocols, 2^{nd} edition; Guisan ed. (2006) Immobilization of Enzymes and Cells; Perbal (1988) A Practical Guide to Molecular Cloning, 2^{nd} edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4^{th} edition; and Herzenberg et al. eds (1996) Weir's Handbook of Experimental Immunology, 5^{th} edition.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the j-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDHR and light chain regions labeled CDLR). Thus, a CDHR3 is the CDR3 from the variable domain of the heavy chain of the antibody in which it is found, whereas a CDLR1 is the CDR1 from the variable domain of the light chain of the antibody in which it is found. A FLT3 antibody will have a specific V_{H} region and the V_{L} region sequence unique to the FLT3 antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein the term “PD-1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-1 sequence as shown herein and/or a suitable binding partner of PD-L1. Non-limiting example sequences of PD-1 are provided herein, such as but not limited to those under the following reference numbers—GCID:GC02M241849; HGNC: 8760; Entrez Gene: 5133; Ensembl: ENSG00000188389; OMIM: 600244; and UniProtKB: Q15116—and the sequence:

MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL
(SEQ ID NO: 18),
and
equivalents thereof.

Non-limiting examples of commercially available antibodies thereto include pembrolizumab (Merck), nivolumab (Bristol-Myers Squibb), pidilizumab (Cure Tech), AMP-224 (GSK), AMP-514 (GSK), PDR001 (Novartis), and cemiplimab (Regeneron and Sanofi).

As used herein the term “PD-L1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-L1 sequence as shown herein and/or an suitable binding partner of PD-1. Non-limiting example sequences of PD-L1 are provided herein, such as but not limited to those under the following reference numbers—GCID: GC09P005450; HGNC: 17635; Entrez Gene: 29126; Ensembl: ENSG00000120217; OMIM: 605402; and UniProtKB: Q9NZQ7—and the sequence:

MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET
(SEQ ID NO: 19),
and
equivalents thereof.

Non-limiting examples of commercially available antibodies thereto include atezolizumab (Roche, avelumab (Merck Soreno and Pfizer), durvalumab (AstraZeneca), BMS-936559 (Bristol-Myers Suibb), and CK-301 (Chekpoint Therapeutics).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “antigen binding domain” refers to any protein or polypeptide domain that can specifically bind to an antigen target.

As used herein, the term “bispecific antibody” refers to an antibody which can bind to two different types of antigen, e.g., has two different antigen binding domains.

As used herein, the term “signal peptide” refers to a peptide sequence that directs the transport and localization of the protein within a cell, e.g., to a certain cell organelle (such as the endoplasmic reticulum) and/or the cell surface. Non-limiting examples of a signal peptide are disclosed herein, e.g., the peptide encoded by the following nucleic acid sequence:

Signal Peptide Sequence:
(SEQ ID NO: 20)
ATGGGATGGAGCTCTATCATCCTCTTCTTGGTAGCAACAGCTACAGGTG
TCCAC,
and
optionally, an equivalent thereof.
Signal Peptide Sequence:
(SEQ ID NO: 21)
MGWSCIILFLVATATGVHS,
and
optionally, an equivalent thereof.
Signal Peptide Sequence:
(SEQ ID NO: 22)
MDWIWRILFLVGAATGAHS,
and
optionally, an equivalent thereof.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸M, 10⁻⁹M, 10⁻¹⁰ M, 10⁻¹¹ M, or 10⁻¹² M.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

As used herein, the term “autologous,” in reference to cells refers to cells that are isolated and infused back into the same subject (recipient or host). “Allogeneic” refers to non-autologous cells.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

“Immune cells” includes, e.g., white blood cells (leukocytes) which are derived from hematopoietic stem cells (HSC) produced in the bone marrow, lymphocytes (T-cells, B-cells, natural killer (NK) cells) and myeloid-derived cells (neutrophil, eosinophil, basophil, monocyte, macrophage, dendritic cells).

As used herein, the term “NK-cell,” also known as natural killer cell, refers to a type of lymphocyte that originates in the bone marrow and play a critical role in the innate immune system. NK-cells provide rapid immune responses against viral-infected cells, tumor cells or other stressed cell, even in the absence of antibodies and major histocompatibility complex on the cell surfaces. NK-cells may either be isolated or obtained from a commercially available source. Non-limiting examples of commercial NK-cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (atcc.org/) and the German Collection of Microorganisms and Cell Cultures (dsmz.de/).

As used herein, the term “B-cell,” refers to a type of lymphocyte in the humoral immunity of the adaptive immune system. B-cells principally function to make antibodies, serve as antigen presenting cells, release cytokines, and develop memory B-cells after activation by antigen interaction. B-cells are distinguished from other lymphocytes, such as T-cells, by the presence of a B-cell receptor on the cell surface. B-cells may either be isolated or obtained from a commercially available source. Non-limiting examples of commercially available B-cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75](ATCC® CRL-2625™), DS-1 (ATCC® CRL-11102™) EB-3 [EB3](ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https://www.dsmz.de/).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™) TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11](ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “recombinant protein” refers to a polypeptide which is produced by recombinant DNA techniques, wherein generally, DNA encoding the polypeptide is inserted into a suitable expression vector which is in turn used to transform a host T-cell to produce the heterologous protein.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspect, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

Regulatory sequences or elements include promoters, enhancer and/or promoter/enhancer combinations. The promoter that regulates expression of the nucleic acid encoding can be a constitutive promoter. Non-limiting examples of constitutive promoters include SFFV, CMV, PKG, MDNU3, SV40, Ef1a, UBC, and CAGG. In one aspect, the enhancer is a Woodchuck post-regulatory element (“WPRE”) (see, e.g., Zufferey, R. et al. (1999) J. Virol. 73(4):2886-2992). The enhancer element can be downstream of the promoter.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “immunoregulatory molecule” may refer to any molecule that may regulate or directly influence immune responses, including but not limited to chemokines such as CCL2, CCL5, CCL14, CCL19, CCL20, CXCL8, CXCL13, and LEC; lymphokines and cytokines such as interleukins (e.g., IL-2, IL-7, IL-12, IL-15, IL-18, IL-21, etc.), interferons α, β and γ, factors stimulating cell growth (e.g., GM-CSF), and other factors (e.g., tumor necrosis factors, DC-SIGN, MlP1α, MlP1β, TGF-β or TNF); factors that provide co-stimulatory signals for T-cell activation such as B7 molecules and CD40; accessory molecules such as CD83; proteins involved in antigen processing and presentation such as TAP1/TAP2 transporter proteins, proteosome molecules such as LMP2 and LMP7, heat shock proteins such as gp96, HSP70 and HSP90, and MHC or HLA molecules; and biological equivalents thereof. Non-limiting examples of immunoregulatory molecules are disclosed herein.

As used herein, the term “B7.1” (also known as B7; BB1; B7-1; CD80; LAB7; CD28LG; CD28LG1) refers to a specific molecule associated with this name and any other molecules that have analogous biological function that share at least 80% amino acid sequence identity, preferably 90% sequence identity, more preferably at least 95% sequence identity with B7.1. Examples of the B7.1 sequence are provided herein. In addition, the sequences associated with GenBank Accession Nos. NM_005191.3 and NP_005182.1 are exemplary. A non-limiting example includes NP_005182.1:

(SEQ ID NO: 23)
MGHTRRQGTS PSKCPYLNFF QLLVLAGLSH FCSGVIHVTK
EVKEVATLSC GHNVSVEELA QTRIYWQKEK KMVLTMMSGD
MNIWPEYKNR TIFDITNNLS IVILALRPSD EGTYECVVLK
YEKDAFKREH LAEVTLSVKA DFPTPSISDF EIPTSNIRRI
ICSTSGGFPE PHLSWLENGE ELNAINTTVS QDPETELYAV
SSKLDFNMTT NHSFMCLIKY GHLRVNQTFN WNTTKQEHFP
DNLLPSWAIT LISVNGIFVI CCLTYCFAPR CRERRRNERL
RRESVRPV

In some embodiments where B7.1 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com/).

As used herein, the term “CCL19” (also known as ELC; CKb11; MIP3B; MIP-3b; SCYA19) refers to a specific molecule associated with this name and any other molecules that have analogous biological function that share at least 80% amino acid sequence identity, preferably 90% sequence identity, more preferably at least 95% sequence identity with CCL19. Examples of the CCL19 sequence are provided herein. In addition, the sequences associated with GenBank Accession Nos. NC_000009.11 NC_018920.2 NT_008413.19, NP_006265.1 are exemplary. A non-limiting example includes NP_006265.1:

(SEQ ID NO: 24)
MALLLALSLL VLWTSPAPTL SGTNDAEDCC LSVTQKPIPG
YIVRNFHYLL IKDGCRVPAV VFTTLRGRQL CAPPDQPWVE
RIIQRLQRTS AKMKRRSS

In some embodiments where CCL19 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com/).

As used herein, the term “CCL20” (also known as CKb4; LARC; ST38; MIP3A; Exodus; MIP-3a; SCYA20; MIP-3-alpha) refers to a specific molecule associated with this name and any other molecules that have analogous biological function that share at least 80% amino acid sequence identity, preferably 90% sequence identity, more preferably at least 95% sequence identity with CCL20. Examples of the CCL20 sequence are provided herein. In addition, the sequences associated with GenBank Accession Nos. NC_000002.11 NC_018913.2 NT_005403.18, NP_001123518.1, and NP_004582.1 are exemplary. Non-limiting examples include NP_004582.1:

(SEQ ID NO: 25)
MCCTKSLLLA ALMSVLLLHL CGESEAASNF DCCLGYTDRI
LHPKFIVGFT RQLANEGCDI NAIIFHTKKK LSVCANPKQT
WVKYIVRLLS KKVKNM

### and NP_001123518.1:

(SEQ ID NO: 26)
MCCTKSLLLA ALMSVLLLHL CGESEASNFD CCLGYTDRIL
HPKFIVGFTR QLANEGCDIN AIIFHTKKKL SVCANPKQTW
VKYIVRLLSK KVKNM

In some embodiments where CCL20 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address: linscottsdirectory.com, last accessed on Jun. 20, 2019).

As used herein, the term “CD40L” (also known as IGM; IMD3; TRAP; gp39; CD154; CD40LG; HIGM1; T-BAM; TNFSF5; hCD40L) refers to a specific molecule associated with this name and any other molecules that have analogous biological function that share at least 80% amino acid sequence identity, preferably 90% sequence identity, more preferably at least 95% sequence identity with CD40L. Examples of the CD40L sequence are provided herein. In addition, the sequences associated with GenBank Accession Nos. NC_000023.10, NC_018934.2, NT_011786.17, NP_000065.1 are exemplary. A non-limiting example includes NP_000065.1:

(SEQ ID NO: 27)
MIETYNQTSP RSAATGLPIS MKIFMYLLTV FLITQMIGSA
LFAVYLHRRL DKIEDERNLH EDFVFMKTIQ RCNTGERSLS
LLNCEEIKSQ FEGFVKDIML NKEETKKENS FEMQKGDQNP
QIAAHVISEA SSKTTSVLQW AEKGYYTMSN NLVTLENGKQ
LTVKRQGLYY IYAQVTFCSN REASSQAPFI ASLCLKSPGR
FERILLRAAN THSSAKPCGQ QSIHLGGVFE LQPGASVFVN
VTDPSQVSHG TGFTSFGLLK L

In some embodiments where CD40L is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address at linscottsdirectory.com, last accessed on Jun. 20, 2019).

As used herein, the term “CD137L” (also known as TNFSF9; 4-1BB-L) refers to a specific molecule associated with this name and any other molecules that have analogous biological function that share at least 80% amino acid sequence identity, preferably 90% sequence identity, more preferably at least 95% sequence identity with CD137L. Examples of the CD137L sequence are provided herein. In addition, the protein associated with GenBank Accession Nos. NC_000019.9, NT_011295.12, NC_018930.2, and NP_003802.1 are exemplary. A non-limiting example includes NP_003802.1:

(SEQ ID NO: 28)
MEYASDASLD PEAPWPPAPR ARACRVLPWA LVAGLLLLLL
LAAACAVFLA CPWAVSGARA SPGSAASPRL REGPELSPDD
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE

In some embodiments where CD137L is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address at: linscottsdirectory.com, last accessed on Jun. 20, 2019).

As used herein, the term “GITRL” (also known as TNFSF18; TL6; AITRL; hGITRL) refers to a specific molecule associated with this name and any other molecules that have analogous biological function that share at least 80% amino acid sequence identity, preferably 90% sequence identity, more preferably at least 95% sequence identity with GITRL. Examples of the GITRL sequence are provided herein. In addition, the protein associated with GenBank Accession Nos. NC_000001.10, NC_018912.2, NT_004487.20, and NP_005083.2 are exemplary. A non-limiting example includes NP_005083.2:

(SEQ ID NO: 29)
MTLHPSPITC EFLESTALIS PKMCLSHLEN MPLSHSRTQG
AQRSSWKLWL FCSIVMLLFL CSFSWLIFIF LQLETAKEPC
MAKFGPLPSK WQMASSEPPC VNKVSDWKLE ILQNGLYLIY
GQVAPNANYN DVAPFEVRLY KNKDMIQTLT NKSKIQNVGG
TYELHVGDTI DLIFNSEHQV LKNNTYWGII LLANPQFIS

In some embodiments where GITRL is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address at linscottsdirectory.com, last accessed on Jun. 20, 2019).

As used herein, the term “GM-CSF” (also known as granulocyte-macrophage colony stimulating factor; CSF2) refers to a specific molecule associated with this name and any other molecules that have analogous biological function that share at least 80% amino acid sequence identity, preferably 90% sequence identity, more preferably at least 95% sequence identity with GM-CSF. Examples of the GM-CSF sequence are provided herein. In addition, the protein sequence associated with UniProt Reference No. P04141—CSF2_HUMAN:

(SEQ ID NO: 30)
MWLQSLLLLG TVACSISAPA RSPSPSTQPW EHVNAIQEAR
RLLNLSRDTA AEMNETVEVI SEMFDLQEPT CLQTRLELYK
QGLRGSLTKL KGPLTMMASH YKQHCPPTPE TSCATQIITF
ESFKENLKDF LLVIPFDCWE PVQE

In some embodiments where GM-CSF is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com).

As used herein, the term “IL-12” (also known as “interleukin 12”) refers to a specific molecule associated with this name and any other molecules that have analogous biological function that share at least 80% amino acid sequence identity, preferably 90% sequence identity, more preferably at least 95% sequence identity with IL-12. Examples of the IL-12 sequence are provided herein, and include but are not limited to mature form IL-12 and variants and fragments thereof, such as single chain IL-12, IL-12A (GenBank Accession Nos. NC_000003.11 NT_005612.17 NC_018914.2), and IL-12B (GenBank Accession Nos. NC_000005.9 NC_018916.2 NT_023133.14). The protein sequences associated with the sequences disclosed in U.S. Pat. No. 8,556,882 are exemplary. In some embodiments where IL-12 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com).

As used herein, the term “IL-2” (also known as “interleukin 2”) refers to a specific molecule associated with this name and any other molecules that have analogous biological function that share at least 80% amino acid sequence identity, preferably 90% sequence identity, more preferably at least 95% sequence identity with IL-2. A non-limiting example below provides the full length sequence of native human IL-2:

(SEQ ID NO: 31)
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT

The term “low-toxicity IL-2” refers to a modified version of IL-2 exhibiting analogous biological function but lower toxicity when administered to a subject. In some embodiments, low-toxicity IL-2 comprises a mutation with reduced vasopermeability compared to wild type IL-2. U.S. Pat. No. 7,371,371 discloses exemplary mutations in the permeability enhancing region of wild type IL-2 between amino acid positions 22 to 58 of human IL-2. Non-limiting examples include a mutation of R to W at position 38 in the human sequence. U.S. Pat. No. 7,371,371 further discloses low-toxicity IL-2 comprising a mutation at one or more positions outside the permeability enhancing region of IL-2.

As used herein, the term “IL-15” (also known as “interleukin 15”) refers to a specific molecule associated with this name and any other molecules that have analogous biological function that share at least 80% amino acid sequence identity, preferably 90% sequence identity, more preferably at least 95% sequence identity with IL-15. Examples of the IL-15 sequence are provided herein. In addition, the protein sequences associated with GenBank Accession Nos. NC_000004.11, NC_018915.2, NT_016354.20, NP_000576.1, NP_751915.1 are exemplary. In some embodiments where 11-15 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “IL-18” (also known as “interleukin 18,” IGIF, “interleukin 1 gamma,” IL1F4, IFN-Gamma-Inducing Factor, IL-1g,) refers to a specific molecule associated with this name and any other molecules that have analogous biological function that share at least 80% amino acid sequence identity, preferably 90% sequence identity, more preferably at least 95% sequence identity with IL-18. Examples of the IL-18 sequence are provided herein. In addition, the protein sequences associated with GenBank Accession Nos. NC_000011.9, NC_018922.2, NT_033899.9, NP_001230140.1, NP_001553.1 are exemplary. In some embodiments where IL-18 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “IL-21” (also known as “interleukin 21”; Za11; CVID11) refers to a specific molecule associated with this name and any other molecules that have analogous biological function that share at least 80% amino acid sequence identity, preferably 90% sequence identity, more preferably at least 95% sequence identity with IL-21. Examples of the IL-21 sequence are provided herein. In addition, the protein sequences associated with GenBank Accession Nos. NC_000004.11, NC_018915.2, NT_016354.20, are exemplary. In some embodiments where 11-21 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “LEC” (also known as CCL16; LMC; NCC4; CKb12; HCC-4; LCC-1; Mtn-1; NCC-4; SCYL4; ILINCK; SCYA16) refers to a specific molecule associated with this name and any other molecules that have analogous biological function that share at least 80% amino acid sequence identity, preferably 90% sequence identity, more preferably at least 95% sequence identity with LEC. Examples of the LEC sequence are provided herein. In addition, the protein sequences associated with GenBank Accession Nos. NC_000017.10, NT_010783.16, NT_187614.1, NC_018928.2, NP_004581.1 are exemplary. A non-limiting example includes NP_004581.1:

(SEQ ID NO: 32)
MKVSEAALSL LVLILIITSA SRSQPKVPEW VNTPSTCCLK
YYEKVLPRRL VVGYRKALNC HLPAIIFVTK RNREVCTNPN
DDWVQEYIKD PNLPLLPTRN LSTVKIITAK NGQPQLLNSQ

In some embodiments where LEC is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “OX40L” (also known as TNFSF4; GP34; CD252; TXGP1; CD134L; OX-40L) refers to a specific molecule associated with this name and any other molecules that have analogous biological function that share at least 80% amino acid sequence identity, preferably 90% sequence identity, more preferably at least 95% sequence identity with OX40L. Examples of the OX40L sequence are provided herein. In addition, the protein sequences associated with GenBank Accession Nos. NC_000001.10, NT_004487.20, NC_018912.2, NP_003317.1 are exemplary.

A non-limiting example includes NP_003317.1:

(SEQ ID NO: 33)
MERVQPLEEN VGNAARPRFE RNKLLLVASV IQGLGLLLCF
TYICLHESAL QVSHRYPRIQ SIKVQFTEYK KEKGFILTSQ
KEDEIMKVQN NSVIINCDGF YLISLKGYFS QEVNISLHYQ
KDEEPLFQLK KVRSVNSLMV ASLTYKDKVY LNVTTDNTSL
DDFHVNGGEL ILIHQNPGEF CVL

In some embodiments where OX40L is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “FLT3” refers to a receptor-type tyrosine-protein kinase FLT3 associated with this name, any of its alternate names (Fms-Related Tyrosine Kinase, Stem Cell Tyrosine Kinase, Fms-Like Tyrosine Kinase, FL Cytokine Receptor, CD135 Antigen, EC 2.7.10.1, CD135, FLK-2, STK1, FLK2, Growth Factor Receptor Tyrosine Kinase Type III, Receptor-Type Tyrosine-Protein Kinase FLT3, Fetal Liver Kinase 2, Fetal Liver Kinase-2, EC 2.7.10, FLT-3, STK-1) or UniProt Accession No. P36888 and any other molecules that have analogous biological function that share at least 80% amino acid sequence identity, preferably 90% sequence identity, or alternatively at least 95% sequence identity with FLT3 and any variant or isoform thereof. Non-limiting examples of FLT3 include:

Human FLT3 Isoform 1,
(SEQ ID NO: 34)
MPALARDGGQLPLLVVFSAMIFGTITNQDLPVIKCVLINHKNNDSSVGKSSSYPMVSE
SPEDLGCALRPQSSGTVYEAAAVEVDVSASITLQVLVDAPGNISCLWVFKHSSLNCQ
PHFDLQNRGVVSMVILKMTETQAGEYLLFIQSEATNYTILFTVSIRNTLLYTLRRPYFR
KMENQDALVCISESVPEPIVEWVLCDSQGESCKEESPAVVKKEEKVLHELFGTDIRCC
ARNELGRECTRLFTIDLNQTPQTTLPQLFLKVGEPLWIRCKAVHVNHGFGLTWELEN
KALEEGNYFEMSTYSTNRTMIRILFAFVSSVARNDTGYYTCSSSKHPSQSALVTIVEK
GFINATNSSEDYEIDQYEEFCFSVRFKAYPQIRCTWTFSRKSFPCEQKGLDNGYSISKF
CNHKHQPGEYIFHAENDDAQFTKMFTLNIRRKPQVLAEASASQASCFSDGYPLPSWT
WKKCSDKSPNCTEEITEGVWNRKANRKVFGQWVSSSTLNMSEAIKGFLVKCCAYNS
LGTSCETILLNSPGPFPFIQDNISFYATIGVCLLFIVVLTLLICHKYKKQFRYESQLQMV
QVTGSSDNEYFYVDFREYEYDLKWEFPRENLEFGKVLGSGAFGKVMNATAYGISKT
GVSIQVAVKMLKEKADSSEREALMSELKMMTQLGSHENIVNLLGACTLSGPIYLIFE
YCCYGDLLNYLRSKREKFHRTWTEIFKEHNFSFYPTFQSHPNSSMPGSREVQIHPDSD
QISGLHGNSFHSEDEIEYENQKRLEEEEDLNVLTFEDLLCFAYQVAKGMEFLEFKSCV
HRDLAARNVLVTHGKVVKICDFGLARDIMSDSNYVVRGNARLPVKWMAPESLFEGI
YTIKSDVWSYGILLWEIFSLGVNPYPGIPVDANFYKLIQNGFKMDQPFYATEEIYIIMQ
SCWAFDSRKRPSFPNLTSFLGCQLADAEEAMYQNVDGRVSECPHTYQNRRPFSREM
DLGLLSPQAQVEDS,
and
optionally an equivalent thereof.
Human FLT3 Isoform 2:
(SEQ ID NO: 35)
MPALARDGGQLPLLVVFSAMIFGTITNQDLPVIKCVLINHKNNDSSVGKSSSYPMVSE
SPEDLGCALRPQSSGTVYEAAAVEVDVSASITLQVLVDAPGNISCLWVFKHSSLNCQ
PHFDLQNRGVVSMVILKMTETQAGEYLLFIQSEATNYTILFTVSIRNTLLYTLRRPYFR
KMENQDALVCISESVPEPIVEWVLCDSQGESCKEESPAVVKKEEKVLHELFGTDIRCC
ARNELGRECTRLFTIDLNQTPQTTLPQLFLKVGEPLWIRCKAVHVNHGFGLTWELEN
KALEEGNYFEMSTYSTNRTMIRILFAFVSSVARNDTGYYTCSSSKHPSQSALVTIVEK
GFINATNSSEDYEIDQYEEFCFSVRFKAYPQIRCTWTFSRKSFPCEQKGLDNGYSISKF
CNHKHQPGEYIFHAENDDAQFTKMFTLNIRRKPQVLAEASASQASCFSDGYPLPSWT
WKKCSDKSPNCTEEITEGVWNRKANRKVFGQWVSSSTLNMSEAIKGFLVKCCAYNS
LGTSCETILLNSPGPFPFIQDNISFYATIGVCLLFIVVLTLLICHKYKKQFRYESQLQMV
QVTGSSDNEYFYVDFREYEYDLKWEFPRENLEFGKVLGSGAFGKVMNATAYGISKT
GVSIQVAVKMLKEKADSSEREALMSELKMMTQLGSHENIVNLLGACTLSGPIYLIFE
YCCYGDLLNYLRSKREKFHRTWTEIFKEHNFSFYPTFQSHPNSSMPGSREVQIHPDSD
QISGLHGNSFHSEDEIEYENQKRLEEEEDLNVLTFEDLLCFAYQVAKGMEFLEFKSAR
LPVKWMAPESLFEGIYTIKSDVWSYGILLWEIFSLGVNPYPGIPVDANFYKLIQNGFK
MDQPFYATEEIYIIMQSCWAFDSRKRPSFPNLTSFLGCQLADAEEAMYQNVDGRVSE
CPHTYQNRRPFSREMDLGLLSPQAQVEDS,
and
optionally an equivalent thereof.

As used herein, the term FLT3-1 refers to an antibody comprising an amino acid sequence with CDRs that share at least 70%, or alternatively at least 80% amino acid sequence identity, preferably 90% sequence identity, more preferably at least 95% sequence identity with any one of the CDRs encoded in the heavy and light chain polynucleotide sequences disclosed herein below, preferably at least one of the CDR3 regions, most preferably both of the CDR3 regions, disclosed below. The amino acid sequences of said CDR regions are also disclosed herein below.

FLT3-1 Heavy Chain Variable Region Sequence:
(SEQ ID NO: 36)
CAGGTCCAACTGCAGCAGCCTGGGGCTGAGCTTGTGAAGCCTGGGGCTTCATTGA
AGCTGTCCTGCAAGTCTTCCGGGTACACCTTCACCAGCTACTGGATGCACTGGGT
GAGGCAGAGGCCTGGACATGGCCTTGAGTGGATCGGAGAGATTGATCCTTCTGA
CAGTTATAAAGACTACAATCAGAAGTTCAAGGACAAGGCCACATTGACTGTGGA
CAGATCCTCCAACACAGCCTACATGCACCTCAGCAGCCTGACATCTGATGACTCT
GCGGTCTATTATTGTGCAAGAGCGATTACGACGACCCCCTTTGACTTCTGGGGCC
AAGGCACCACTCTCACAGTCTCCTCA,
and
optionally an equivalent thereof.
FLT3-1 Light Chain Variable Region Sequence:
(SEQ ID NO: 37)
GATATTGTGCTAACTCAGTCTCCAGCCACCCTGTCTGTGACTCCAGGAGATAGCG
TCAGTCTTTCCTGCAGGGCCAGCCAGAGTATTAGCAACAACCTACACTGGTATCA
ACAAAAATCACATGAGTCTCCAAGGCTTCTCATCAAGTATGCTTCCCAGTCCATC
TCTGGGATCCCCTCCAGGTTCAGTGGCAGTGGATCAGGGACAGATTTCACTCTCA
GTATCAACAGTGTGGAGACTGAAGATTTTGGAGTGTATTTCTGTCAACAGAGTAA
CACCTGGCCGTACACGTTCGGAGGGGGGACCAAGCTGGAAATAAAACGG,
and
optionally an equivalent thereof.
FLT3-1 CDHR1:
(SEQ ID NO: 1)
SYWMH,
and
optionally an equivalent thereof.
FLT3-1 CDHR2:
(SEQ ID NO: 3)
EIDPSDSYKDYNQKFKD,
and
optionally an equivalent thereof.
FLT3-1 CDHR3:
(SEQ ID NO: 5)
AITTTPFDF,
and
optionally an equivalent thereof.
FLT3-1 CDLR1:
(SEQ ID NO: 7)
RASQSISNNLH,
and
optionally an equivalent thereof.
FLT3-1 CDLR2:
(SEQ ID NO: 9)
YASQSIS,
and
optionally an equivalent thereof.
FLT3-1 CDLR3:
(SEQ ID NO: 11)
QQSNTWPYT,
and
optionally an equivalent thereof.

As used herein, the term FLT3-2 refers to an antibody comprising an amino acid sequence with CDRs that share at least 70%, or alternatively at least 80% amino acid sequence identity, preferably 90% sequence identity, more preferably at least 95% sequence identity with any one of the CDRs encoded in the heavy and light chain polynucleotide sequences disclosed herein below, preferably at least one of the CDR3 regions, most preferably both of the CDR3 regions, disclosed below. The amino acid sequences of said CDR regions are also disclosed herein below.

FLT3-2 Heavy Chain Variable Region Sequence:
(SEQ ID NO: 38)
CAGGTGCAGCTGAAGCAGTCAGGACCTGGCCTAGTGCAGCCCTCACAGAGCCTG
TCCATCACCTGCACAGTCTCTGGTTTCTCATTAACTAACTATGGTTTACACTGGGT
TCGCCAGTCTCCAGGAAAGGGCCTGGAGTGGCTGGGAGTGATATGGAGTGGTGG
AAGCACAGACTATAATGCAGCTTTCATATCCAGACTGAGCATCAGCAAGGACAA
CTCCAAGAGCCAAGTTTTCTTTAAAATGAACAGTCTGCAGGCTGATGACACAGCC
ATATACTACTGTGCCAGAAAAGGAGGGATCTACTATGCTAACCATTACTATGCTA
TGGACTACTGGGGTCAAGGAACCTCAGTCACCGTCTCCTCA,
and
optionally an equivalent thereof.
FLT3-2 Light Chain Variable Region Sequence:
(SEQ ID NO: 39)
GACATTGTGATGACACAGTCTCCATCCTCCCTGAGTGTGTCAGCAGGAGAGAAG
GTCACTATGAGCTGCAAGTCCAGTCAGAGTCTGTTAAACAGTGGAAATCAAAAG
AACTATATGGCCTGGTATCAGCAGAAACCAGGGCAGCCTCCTAAACTGTTGATCT
ACGGGGCATCCACTAGGGAATCTGGGGTCCCTGATCGCTTCACAGGCAGTGGAT
CTGGAACCGATTTCACTCTTACCATCAGCAGTGTGCAGGCTGAAGACCTGGCAGT
TTATTACTGTCAGAATGATCATAGTTATCCGCTCACGTTCGGTGCTGGGACCAAG
CTGGAGCTGAAACGG,
and
optionally an equivalent thereof.
FLT3-2 CDHR1:
(SEQ ID NO: 2)
NYGLH,
and
optionally an equivalent thereof.
FLT3-2 CDHR2:
(SEQ ID NO: 4)
VIWSGGSTDYNAAFIS,
and
optionally an equivalent thereof.
FLT3-2 CDHR3:
(SEQ ID NO: 6)
GGIYYANHYYAMDY,
and
optionally an equivalent thereof.
FLT3-2 CDLR1:
(SEQ ID NO: 8)
KSSQSLLNSGNQKNYM,
and
optionally an equivalent thereof.
FLT3-2 CDLR2:
(SEQ ID NO: 10)
GASTRES,
and
optionally an equivalent thereof.
FLT3-2 CDLR3:
(SEQ ID NO: 12)
QNDHSYPLT,
and
optionally an equivalent thereof.

As used herein, the term “FLT3 inhibitor” refers to a molecule that binds FLT3 and decreases its activity. Not to be bound by theory, it is believed that such FLT3 inhibitors can increase surface FLT3 expression on cells. Non-limiting examples of FLT3 inhibitors include gilteritinib (Astellas), quizaritinib (Ambit Biosciences), midostaurin (Novartis), sorafenib (Bayer and Onxy Pharmaceuticals), sunitinib (Pfizer), lestarutinib (Cephalon), FF-10101 (Fuijfilm), dovitinib (Novartis or Oncology Venture), and equivalents thereof such as but not limited to salts and hydrates. Exemplary structures for some of these exemplary FLT3 inhibitors are provided herein below:

| Compound Name | Chemical Structure |
|---|---|
| Gilteritinib (CID 49803313) | 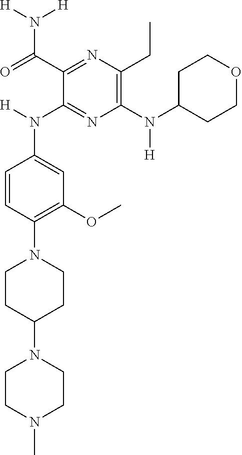 [SMILES: CCc1nc(C(N)=O)c(Nc2ccc(N3CCC(N4CCN(C)CC4)CC3)c(OC)c2)nc1NC1CCOCC1] |
| Gilteritinib Fumarate (CID 76970819) | 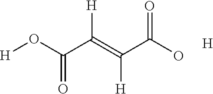 |
| | 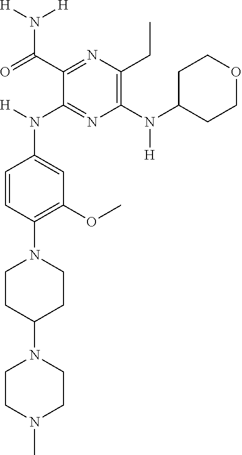 [SMILES: CCc1nc(C(N)=O)c(Nc2ccc(N3CCC(N4CCN(C)CC4)CC3)c(OC)c2)nc1NC1CCOCC1] |
| | 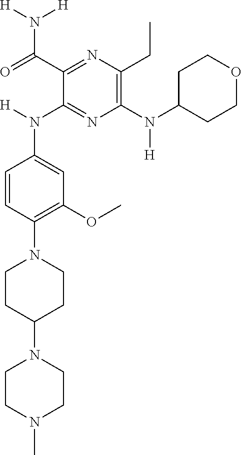 [SMILES: CCc1nc(C(N)=O)c(Nc2ccc(N3CCC(N4CCN(C)CC4)CC3)c(OC)c2)nc1NC1CCOCC1] |
| Quizaritinib (CID 24889392) | 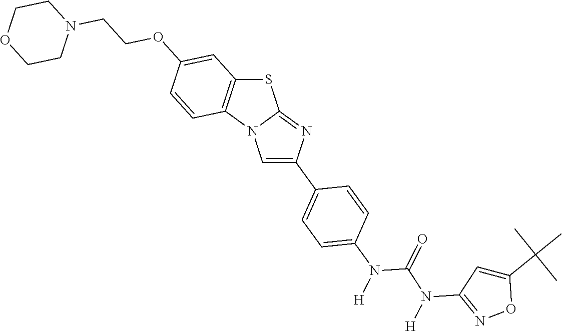 [SMILES: CC(C)(C)c1cc(NC(=O)Nc2ccc(-c3cn4c(n3)sc3cc(OCCN5CCOCC5)ccc34)cc2)no1] |
| Quizartinib Dihydrochloride (CID 25184035) | 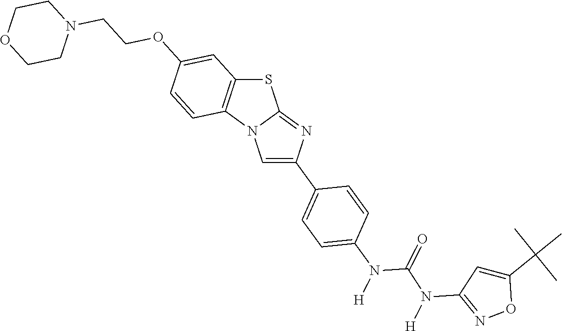 [SMILES: CC(C)(C)c1cc(NC(=O)Nc2ccc(-c3cn4c(n3)sc3cc(OCCN5CCOCC5)ccc34)cc2)no1] |
| | Cl—H |
| | Cl—H |
| Midostaurin (CID 9829523) | 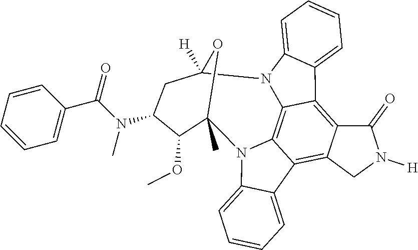 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] |
| Midostaurin Hydrate (CID 71311854) | 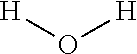 |
| | 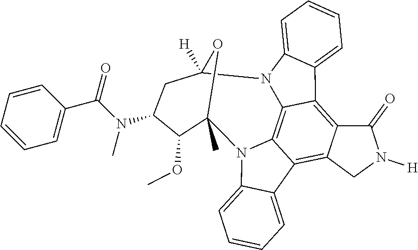 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] |
| Sorafenib (CID 216239) | 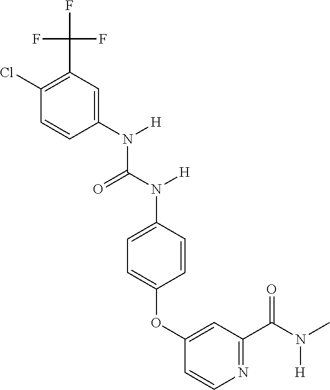 [SMILES: CNC(=O)c1cc(Oc2ccc(NC(=O)Nc3ccc(Cl)c(C(F)(F)F)c3)cc2)ccn1] |
| Sorafenib tosylate (CID 406563) | 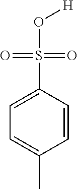 |
| | 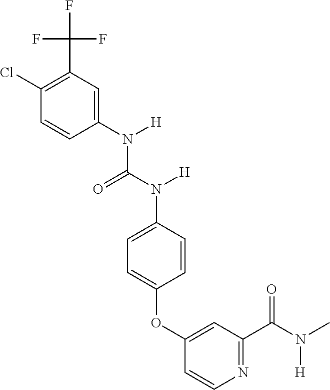 [SMILES: CNC(=O)c1cc(Oc2ccc(NC(=O)Nc3ccc(Cl)c(C(F)(F)F)c3)cc2)ccn1] |
| Sorafenib sulphate (CID 86672519) | 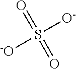 |
| | 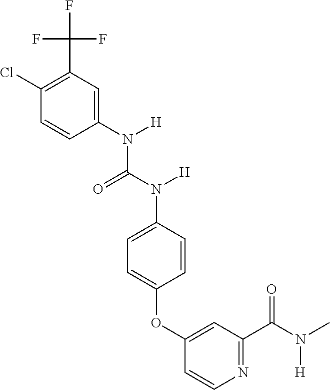 [SMILES: CNC(=O)c1cc(Oc2ccc(NC(=O)Nc3ccc(Cl)c(C(F)(F)F)c3)cc2)ccn1] |
| Sorafenib hydrobromide (CID 44599974) | 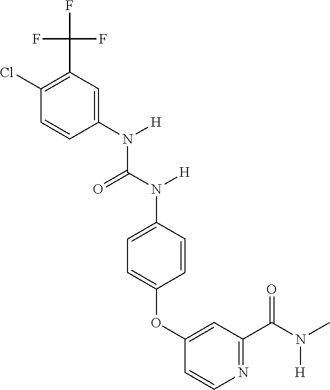 [SMILES: CNC(=O)c1cc(Oc2ccc(NC(=O)Nc3ccc(Cl)c(C(F)(F)F)c3)cc2)ccn1] |
| | Br—H |
| Sorafenib hydrochloride (CID 44599975) | Cl—H |
| | 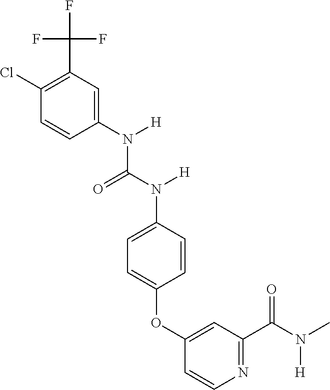 [SMILES: CNC(=O)c1cc(Oc2ccc(NC(=O)Nc3ccc(Cl)c(C(F)(F)F)c3)cc2)ccn1] |
| Sunitinib (CID 5329102) | 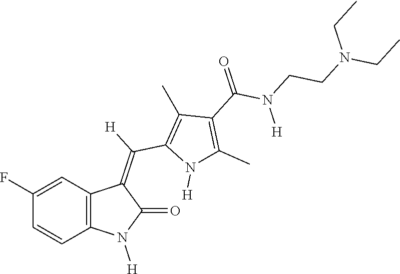 [SMILES: CCN(CC)CCNC(=O)c1c(C)[nH]c(/C=C2\C(=O)Nc3ccc(F)cc32)c1C] |
| Sunitinib Malate (CID 6456015) | 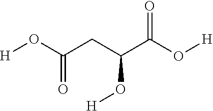 |
| | 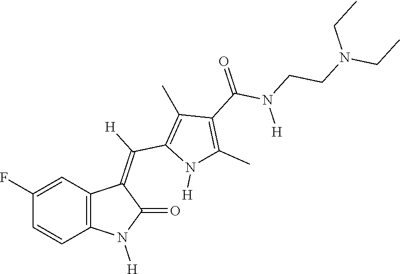 [SMILES: CCN(CC)CCNC(=O)c1c(C)[nH]c(/C=C2\C(=O)Nc3ccc(F)cc32)c1C] |
| Lestarutinib (CID 126565) | 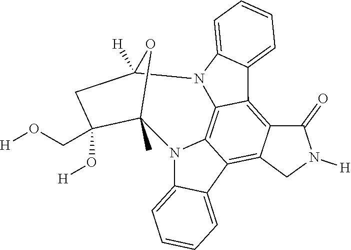 [SMILES: C[C@]12O[C@H](C[C@]1(O)CO)n1c3ccccc3c3c4c(c5c6ccccc6n2c5c31)CNC4=O] |
| Lestarutinib Hydrate (CID 45111934) | 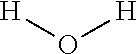 |
| | 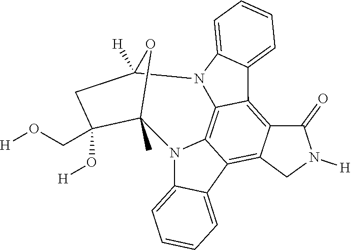 [SMILES: C[C@]12O[C@H](C[C@]1(O)CO)n1c3ccccc3c3c4c(c5c6ccccc6n2c5c31)CNC4=O] |
| Lestarutinib Methanolate (CID 131738508) | —O |
| | 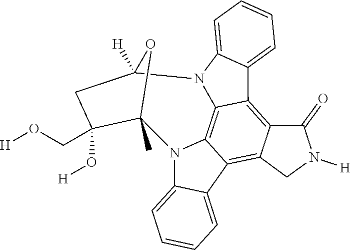 [SMILES: C[C@]12O[C@H](C[C@]1(O)CO)n1c3ccccc3c3c4c(c5c6ccccc6n2c5c31)CNC4=O] |
| Dovitinib (CID 9886808) | 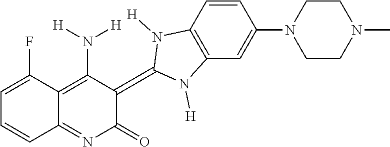 [SMILES: CN1CCN(c2ccc3c(c2)N/C(=C2\C(=O)N=c4cccc(F)c4=C2N)N3)CC1] |
| Dovitinib Lactate (CID 44150621) | 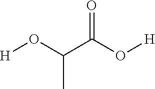 |
| | 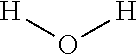 |
| | 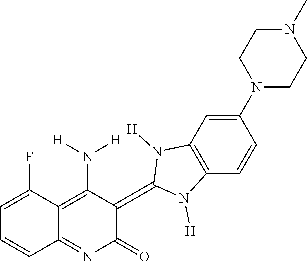 [SMILES: CN1CCN(c2ccc3c(c2)N/C(=C2/C(=O)N=c4cccc(F)c4=C2N)N3)CC1] |
| Dovitinib Dilactic Acid (CID 66553150) | 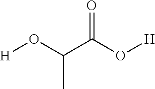 |
| | 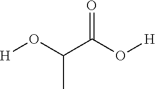 |
| | 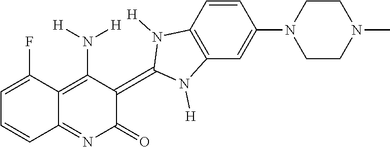 [SMILES: CN1CCN(c2ccc3c(c2)N/C(=C2\C(=O)N=c4cccc(F)c4=C2N)N3)CC1] |

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non-limiting exemplary polynucleotide sequences that encode for components of each domain are disclosed herein, e.g.:

Hinge domain: IgG1 heavy chain hinge sequence:
(SEQ ID NO: 40)
CTCGAGCCCAAATCTTGTGACAAAACTCACACATGCCCACCGTGCCCG,
and
optionally an equivalent thereof.
Hinge domain: IgG1 heavy chain hinge amino acid sequence:
(SEQ ID NO: 41)
LEPKSCDKTHTCPPCP,
and
optionally an equivalent thereof.
Transmembrane domain: CD28 transmembrane region:
(SEQ ID NO: 42)
TTTTGGGTGCTGGTGGTGGTTGGTGGAGTCCTGGCTTGCTATAGCTTGCTAGTAA
CAGTGGCCTTTATTATTTTCTGGGTG,
and
optionally an equivalent thereof.
Transmembrane domain: CD28 transmembrane region amino acid sequence:
(SEQ ID NO: 43)
FWVLVVVGGVLACYSLLVTVAFIIFWV,
and
optionally an equivalent thereof.
Intracellular domain: 4-1BB co-stimulatory signaling region:
(SEQ ID NO: 44)
AACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG,
and
optionally an equivalent thereof.
Intracellular domain: 4-1BB co-stimulatory signaling region amino acid sequence:
(SEQ ID NO: 45)
KRGRKKLLYIFKQPFMRPVQTTQEEDGCSCRFPEEEEGGCEL,
and
optionally an equivalent thereof.
Intracellular domain: CD28 co-stimulatory signaling region:
(SEQ ID NO: 46)
AGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCG
CAGCCTATCGCTCC,
and
optionally an equivalent thereof.
Intracellular domain: CD28 co-stimulatory signaling region amino acid sequence:
(SEQ ID NO: 47)
RSKRSRLLHSDYMNMTPRRPGPTRKHYQPYAPPRDFAAYRS,
and
optionally an equivalent thereof.
Intracellular domain: CD3 zeta signaling region:
(SEQ ID NO: 48)
AGAGTGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACCC
TCAGGAAGGCCTGTACAATGAACTGCAGAAAGATAAGATGGCGGAGGCCTACAG
TGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGC
CCTGCCCCCTCGCTAA,
and
optionally an equivalent thereof.
Intracellular domain: CD3 zeta signaling region amino acid sequence:
(SEQ ID NO: 49)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALP
PR,
and
optionally an equivalent thereof.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 (SEQ ID NO: 50) flexible polypeptide linker comprising three copies of gly-gly-gly-gly-ser (SEQ ID NO: 51)—represented in single letter sequence notation as GGGGS (SEQ ID NO: 51).

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177.

The sequences associated with the CD8 α hinge domain are provided in Pinto, R. D. et al. (2006) Vet. Immunol. Immunopathol. 110:169-177. Non-limiting examples of such include:

Human CD8 alpha hinge domain:
(SEQ ID NO: 53)
PAKPTTTPAPRPPTPAPTIASQPLSLRPEACRPAAGGAVHTRGLDFACD
IY,
and
optionally an equivalent thereof.
Mouse CD8 alpha hinge domain:
(SEQ ID NO: 54)
KVNSTTTKPVLRTPSPVHPTGTSQPQRPEDCRPRGSVKGTGLDFACDI
Y,
and
optionally an equivalent thereof.
Cat CD8 alpha hinge domain:
(SEQ ID NO: 55)
PVKPTTTPAPRPPTQAPITTSQRVSLRPGTCQPSAGSTVEASGLDLSCD
IY,
and
optionally an equivalent thereof.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain(GenBank Accession No: NP_113726.1) provide additional example sequences of the CD8 α transmembrane domain. The sequences associated with each of the listed accession numbers are provided as follows:

Human CD8 alpha transmembrane domain:
(SEQ ID NO: 56)
IYIWAPLAGTCGVLLLSLVIT,
and
optionally an equivalent thereof.
Mouse CD8 alpha transmembrane domain:
(SEQ ID NO: 57)
IWAPLAGICVALLLSLIITLI,
and
optionally an equivalent thereof.
Rat CD8 alpha transmembrane domain:
(SEQ ID NO: 58)
IWAPLAGICAVLLLSLVITLI,
and
optionally an equivalent thereof.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1, such as the exemplary sequence provided below 4-1BB costimulatory signaling region:

(SEQ ID NO: 45)
KRGRKKLLYIFKQPFMRPVQTTQEEDGCSCRFPEEEEGGCEL,
and
optionally an equivalent thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Patent Application Publication No. 2015/0017141A1 the exemplary polynucleotide sequence provided below.

ICOS costimulatory signaling region:
(SEQ ID NO: 59)
ACAAAAAAGA AGTATTCATC CAGTGTGCAC GACCCTAACG GTGAATACAT
GTTCATGAGA GCAGTGAACA CAGCCAAAAA ATCCAGACTC ACAGATGTGA
CCCTA,
and
optionally an equivalent thereof.
ICOS costimulatory signaling region amino acid sequence:
(SEQ ID NO: 60)
TKKKYSSSVHDPNGEYMFMRAVNTAKKSRLTDVTL,
and
optionally an equivalent thereof.

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Patent Application Publication No. 2012/20148552A1, and include the exemplary sequence provided below.

OX40 costimulatory signaling region:
(SEQ ID NO: 61)
AGGGACCAG AGGCTGCCCC CCGATGCCCA CAAGCCCCCT GGGGGAGGCA
GTTTCCGGAC CCCCATCCAA GAGGAGCAGG CCGACGCCCA CTCCACCCTG
GCCAAGATC,
and
optionally an equivalent thereof.
OX40 costimulatory signaling region amino acid sequence:
(SEQ ID NO: 62)
RDQRLPPDAHKPPGGGSFRTPIQEEQADAHSTLAKI,
and
optionally an equivalent thereof.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98: 2364-2371; Hombach, A. et al. (2001) J Immunol 167: 6123-6131; Maher, J. et al. (2002) Nat. Biotechnol. 20: 70-75; Haynes, N. M. et al. (2002) J Immunol 169: 5780-5786 (2002); Haynes, N. M. et al. (2002) Blood 100: 3155-3163. Non-PGP-88 DNA M limiting examples include residues 114-220 of the below. CD28 Sequence:

(SEQ ID NO: 63)
MLRLLLALNL
FPSIQVTGNK ILVKQSPMLV AYDNAVNLSC KYSYNLFSRE
FRASLHKGLDSAVEVCVVYG NYSQQLQVYS KTGFNCDGKL GNESVTFYLQ
NLYVNQTDIY FCKIEVMYPPPYLDNEKSNG TIIHVKGKHL CPSPLFPGPS
KPFWVLVVVG GVLACYSLLVTVAFIIFWVR SKRSRLLHSD YMNMTPRRPG
PTRKHYQPYA PPRDFAAYRS,
and
equivalents thereof.

As used herein, the term “CD3 zeta signaling domain” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD3 zeta signaling domain sequence as shown herein. Non-limiting example sequences of the CD3 zeta signaling domain are provided in U.S. Pat. No. 8,399,645, e.g.:

(SEQ ID NO: 49)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

As used herein, the term “suicide gene” is a gene capable of inducing cell apoptosis; non-limiting examples include HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). Suicide genes may function along a variety of pathways, and, in some cases, may be inducible by an inducing agent such as a small molecule. For example, the iCasp suicide gene comprises portion of a caspase protein operatively linked to a protein optimized to bind to an inducing agent; introduction of the inducing agent into a cell comprising the suicide gene results in the activation of caspase and the subsequent apoptosis of said cell.

The term “FKBP,” or FK506 binding protein, refers to a family of proteins that have prolyl isomerase activity and are related to the cyclophilins in function. FKBPs have been identified in many eukaryotes from yeast to humans and function as protein folding chaperones for proteins containing proline residues. Along with cyclophilin, FKBPs belong to the immunophilin family. A non-limiting exemplary FKBP is human FKBP12 (also referred to as FKBP1A), UniProt Ref. No. P62942. Further non-limiting examples of FKBP include those provided by GenBank Accession Nos. AH002818, BC119732.1, NM_001199786.1, and NM_054014.3.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 64), wherein X refers to any amino acid generally thought to be self-cleaving.

The term “transduce” or “transduction” as it is applied to the production of chimeric antigen receptor cells refers to the process whereby a foreign nucleotide sequence is introduced into a cell. In some embodiments, this transduction is done via a vector.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, a “cancer” is a disease state characterized by the presence in a subject of cells demonstrating abnormal uncontrolled replication and may be used interchangeably with the term “tumor.” In some embodiments, the cancer is a leukemia or a lymphoma. In certain embodiments, the cancer is acute myeloid leukemia or acute lymphoblastic leukemia. As used herein a “leukemia” is a cancer of the blood or bone marrow characterized by an abnormal increase of immature white blood cells. The specific condition of acute myeloid leukemia (AML)—also referred to as acute myelogenous leukemia or acute myeloblastic leukemia—is a cancer of the myeloid origin blood cells, characterized by the rapid growth of abnormal myeloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired.

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

A “normal cell corresponding to the cancer tissue type” refers to a normal cell from a same tissue type as the cancer tissue. A non-limiting example is a normal leukocyte from a patient, e.g., a patient with leukemia.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, “treatment” excludes prevention. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

“Cytoreductive therapy,” as used herein, includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m² to not more than about 50 J/m², usually about 10 J/m².

As used herein the term “hematopoiesis” refers to a subject's ability to produce blood cells and/or platelets in the bone marrow. The term “normal hematopoiesis” can refer to either a subject's baseline level of hematopoiesis and/or a clinically acceptable threshold for normal hematopoiesis based on the average levels of blood cells and/or platelets produced by a population of subjects that do not have a disease or disorder affecting hematopoiesis, such as but not limited to a cancer of the blood or bone marrow. Thus, as used herein the term “maintain normal hematopoiesis” refers to the subject's ability to maintain the specified normal level during or after an intervention and the term “recover normal hematopoiesis” refers to the subject's ability to revert to the specified normal level during or after an intervention.

As used herein, the term “CD34” refers to a protein expressed on a variety of cells including but not limited to hematopoietic cells and a subpopulation of dendritic cells associated with Gene Cards ID GC01M207880. A non-limiting exemplary protein sequence of human CD34 can be found under UniProt Ref. No. P28906; mouse CD34, UniProt Ref. No. Q64314. “CD34+” cells are those cells detected to have CD34 surface expression. Non-limiting exemplary CD34+ cells include hematopoietic stem cells that are capable of self-renewal, proliferation, and differentiation into progenitors in the myeloid, lymphoid, and erythroid lines found in the Lin−CD34+CD38−CD90+CD45RA− compartment; these cells are critical to engraftment of hematopoietic cells and are also known to be FLT3+. See Bhatia et al. (1997) PNAS 94(10):5230-5235; Notta et al. (2010) Blood 115(18):3074-3077; Kikushige et al. (2008) J. Immunol. 180(11):7358-7367.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, (3-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. In one aspect, a detectable marker excludes naturally fluorescent polynucleotides.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. As used herein, the term “polycistronic” in reference to a vector refers to a vector comprising multiple coding regions (exons), e.g., monocistronic (having one coding region), bicistroinc (having two coding regions), and tricistronic (having three coding regions).

The sequences associated with each of the above listed GenBank Accession Nos., UniProt Reference Nos., other reference ID numbers, and references are herein incorporated by reference.

### LIST OF ABBREVIATIONS

AML: acute myeloid leukemia
ALL: acure lymphoblastic leukemia
CAR: chimeric antigen receptor
iCasp: induced caspase

### MODES FOR CARRYING OUT THE DISCLOSURE

Due to the unprecedented results being recently obtained in B-cell lymphomas and leukemia's using autologous treatment with genetically engineered chimeric antigen receptor (CAR) T-cells (Maude, S. L. et al. (2014) New Engl. J. Med. 371:1507-1517; Porter, D. L. et al. (2011) New Engl. J. Med. 365:725-733), a number of laboratories have begun to apply this approach to solid tumors including ovarian cancer, prostate cancer, and pancreatic tumors. CAR modified T-cells combine the HLA-independent targeting specificity of a monoclonal antibody with the cytolytic activity, proliferation, and homing properties of activated T-cells, but do not respond to checkpoint suppression. Because of their ability to kill antigen expressing targets directly, CAR T-cells are highly toxic to any antigen positive cell or tissue making it a requirement to construct CARs with highly tumor specific antibodies. To date, CAR modified T-cells to human solid tumors have been constructed against the α-folate receptor, mesothelin, MUC-CD, PSMA, and other targets; but most have some off-target expression of antigen in normal tissues. These constructs have not shown the same exceptional results in patients emphasizing the need for additional studies to identify new targets and methods of CAR T-cell construction that can be used against solid tumors and other cancers. This disclosure meets these challenges. Applicant had found that CAR NK-cells or NK-cell in prolonged culture expressed substantial amounts of checkpoint protein PD-1, which is an inhibitory signal on cancer patient NK-cells, whereas the AML blasts expressed PD-L1 on cell surface. From this previous bispecific platforms and other groups, the major concern of bispecific antibody (“biAb”) is the short half-life, limiting the bioavailability and efficacy. Therefore, Applicant sought to overcome this technical limitation and provide a synergistic cytolytic activity against AML through increased engagement, increased activation and antagonizing checkpoints inhibition.

Thus, this disclosure provides one or more isolated polynucleotides and/or vectors encoding a chimeric antigen receptor (CAR) comprising: 1) a binding domain specific to FLT3, that in some aspects, is the antigen binding domain of a FLT3 antibody, and 2) an antigen binding domain that recognizes and binds PD-1 and/or PD-L1. Methods and compositions relating to the use and production thereof are further provided herein.

### Chimeric Antigen Receptors and Uses Thereof

### Components of the CAR

The present disclosure provides chimeric antigen receptors (CAR) that bind to FLT3, the CAR comprising, or consisting essentially of, or consisting of, a cell activation moiety comprising an extracellular, transmembrane, and intracellular domain. The extracellular domain comprises a target-specific binding element otherwise referred to as the antigen binding domain. In one aspect, the intracellular domain or cytoplasmic domain comprises a costimulatory signaling region and a zeta chain portion. The CAR may optionally further comprise a spacer domain of up to 300 amino acids, preferably 10 to 100 amino acids, more preferably 25 to 50 amino acids.

Spacer Domain. The CAR may optionally further comprise a spacer domain of up to 300 amino acids, preferably 10 to 100 amino acids, more preferably 25 to 50 amino acids. For example, the spacer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. A spacer domain may comprise, for example, a portion of a human Fc domain, a CH3 domain, or the hinge region of any immunoglobulin, such as IgA, IgD, IgE, IgG, or IgM, or variants thereof. For example, some embodiments may comprise an IgG4 hinge with or without a S228P, L235E, and/or N297Q mutation (according to Kabat numbering). Additional spacers include, but are not limited to, CD4, CD8, and CD28 hinge regions.

Antigen Binding Domain. The CARs of this disclosure comprise an antigen binding domain of an FLT3 antibody or an antibody (i.e. the complete antibody) that binds FLT3. Monoclonal antibodies that specifically bind FLT3 are commercially available from, for example, Becton Dickinson Biosciences and other commercial sources, e.g., those listed at the web address: biocompare.com/Search-Antibodies/?search=FLT3&said=0. Methods to prepare antigen binding fragments are known in the art and briefly described herein. The antigen binding domains may be from any appropriate species, e.g., sheep or human.

In one aspect, the antigen binding domain comprises the heavy chain variable region and the light chain variable region of a FLT3 antibody. In some embodiments, the antigen binding domain comprises, or consists essentially of, or consists of a fragment of the target-specific antibody (i.e., an anti-FLT3 antibody), for example, an scFv. An scFv region can comprise the variable regions of the heavy (V_{H}) and light chains (V_{L}) of immunoglobulins, connected with a short linker peptide. The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments, the heavy chain variable region comprises, or alternatively consists essentially of, or yet further consists of, the polypeptide encoded by polynucleotide sequence:

(SEQ ID NO: 36)
CAGGTCCAACTGCAGCAGCCTGGGGCTGAGCTTGTGAAGCCTGGGGCTT
CATTGAAGCTGTCCTGCAAGTCTTCCGGGTACACCTTCACCAGCTACTG
GATGCACTGGGTGAGGCAGAGGCCTGGACATGGCCTTGAGTGGATCGGA
GAGATTGATCCTTCTGACAGTTATAAAGACTACAATCAGAAGTTCAAGG
ACAAGGCCACATTGACTGTGGACAGATCCTCCAACACAGCCTACATGCA
CCTCAGCAGCCTGACATCTGATGACTCTGCGGTCTATTATTGTGCAAGA
GCGATTACGACGACCCCCTTTGACTTCTGGGGCCAAGGCACCACTCTCA
CAGTCTCCTCA,
or
an antigen binding fragment or an equivalent
of thereof.

In other embodiments, the heavy chain variable region comprises, or alternatively consists essentially of, or yet further consists of, the polypeptide sequence:

(SEQ ID NO: 65)
QVQLQQPGAELVKPGASLKLSCKSSGYTFTSYWMHWVRQRPGHGLEWIG
EIDPSDSYKDYNQKFKDKATLTVDRSSNTAYMHLSSLTSDDSAVYYCAR
AITTTPFDFWGQGTTLTVSS,
or
an antigen binding fragment or an equivalent
of thereof.

In some embodiments, the heavy chain variable region comprises, or alternatively consists essentially of, or yet further consists of, the polypeptide encoded by polynucleotide sequence disclosed:

(SEQ ID NO: 38)
CAGGTGCAGCTGAAGCAGTCAGGACCTGGCCTAGTGCAGCCCTCACAGA
GCCTGTCCATCACCTGCACAGTCTCTGGTTTCTCATTAACTAACTATGG
TTTACACTGGGTTCGCCAGTCTCCAGGAAAGGGCCTGGAGTGGCTGGGA
GTGATATGGAGTGGTGGAAGCACAGACTATAATGCAGCTTTCATATCCA
GACTGAGCATCAGCAAGGACAACTCCAAGAGCCAAGTTTTCTTTAAAAT
GAACAGTCTGCAGGCTGATGACACAGCCATATACTACTGTGCCAGAAAA
GGAGGGATCTACTATGCTAACCATTACTATGCTATGGACTACTGGGGTC
AAGGAACCTCAGTCACCGTCTCCTCA,
or
an antigen binding fragment or an equivalent
thereof.

In other embodiments, the heavy chain variable region comprises, or alternatively consists essentially of, or yet further consists of, the polypeptide sequence:

(SEQ ID NO: 66)
QVQLKQSGPGLVQPSQSLSITCTVSGFSLTNYGLHWVRQSPGKGLEWLG
VIWSGGSTDYNAAFISRLSISKDNSKSQVFFKMNSLQADDTAIYYCARK
GGIYYANHYYAMDYWGQGTSVTVSS,
or
an antigen binding fragment or an equivalent
of thereof.

In some embodiments, the heavy chain variable region comprises a CDRH1 sequence comprising, or alternatively consisting essentially of, or yet further consisting of, an amino acid sequence beginning with SYWMH (SEQ ID NO: 1), NYGLH (SEQ ID NO: 2), or an equivalent each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH2 sequence comprising, or alternatively consisting essentially of, or yet further consisting of, an amino acid sequence beginning with EIDPSDSYKDYNQKFKD (SEQ ID NO: 3), VIWSGGSTDYNAAFIS (SEQ ID NO: 4), or an equivalent each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH3 sequence comprising, or alternatively consisting essentially of, or yet further consisting of, an amino acid sequence beginning with AITTTPFDF (SEQ ID NO: 5), GGIYYANHYYAMDY (SEQ ID NO: 6), or an equivalent each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises, or alternatively consists essentially of, or yet further consists of, the polypeptide encoded by polynucleotide sequence:

(SEQ ID NO: 37)
GATATTGTGCTAACTCAGTCTCCAGCCACCCTGTCTGTGACTCCAGGAG
ATAGCGTCAGTCTTTCCTGCAGGGCCAGCCAGAGTATTAGCAACAACCT
ACACTGGTATCAACAAAAATCACATGAGTCTCCAAGGCTTCTCATCAAG
TATGCTTCCCAGTCCATCTCTGGGATCCCCTCCAGGTTCAGTGGCAGTG
GATCAGGGACAGATTTCACTCTCAGTATCAACAGTGTGGAGACTGAAGA
TTTTGGAGTGTATTTCTGTCAACAGAGTAACACCTGGCCGTACACGTTC
GGAGGGGGGACCAAGCTGGAAATAAAACGG,
or
an antigen binding fragment thereof or an
equivalent of each thereof.

In other embodiments, the light chain variable region comprises, or alternatively consists essentially of, or yet further consists of, the polypeptide sequence:

(SEQ ID NO: 67)
DIVLTQSPATLSVTPGDSVSLSCRASQSISNNLHWYQQKSHESPRLLIK
YASQSISGIPSRFSGSGSGTDFTLSINSVETEDFGVYFCQQSNTWPYTF
GGGTKLEIKR,
or
an antigen binding fragment or an equivalent
of thereof.

In some embodiments, the light chain variable region comprises, or alternatively consists essentially of, or yet further consists of, the polypeptide encoded by polynucleotide sequence:

(SEQ ID NO: 39)
GACATTGTGATGACACAGTCTCCATCCTCCCTGAGTGTGTCAGCAGGAG
AGAAGGTCACTATGAGCTGCAAGTCCAGTCAGAGTCTGTTAAACAGTGG
AAATCAAAAGAACTATATGGCCTGGTATCAGCAGAAACCAGGGCAGCCT
CCTAAACTGTTGATCTACGGGGCATCCACTAGGGAATCTGGGGTCCCTG
ATCGCTTCACAGGCAGTGGATCTGGAACCGATTTCACTCTTACCATCAG
CAGTGTGCAGGCTGAAGACCTGGCAGTTTATTACTGTCAGAATGATCAT
AGTTATCCGCTCACGTTCGGTGCTGGGACCAAGCTGGAGCTGAAACGG,
or
an antigen binding fragment thereof or an
equivalent of each thereof.

In other embodiments, the light chain variable region comprises, or alternatively consists essentially of, or yet further consists of, the polypeptide sequence:

(SEQ ID NO: 68)
DIVMTQSPSSLSVSAGEKVTMSCKSSQSLLNSGNQKNYMAWYQQKPGQP
PKLLIYGASTRESGVPDRFTGSGSGTDFTLTISSVQAEDLAVYYCQNDH
SYPLTFGAGTKLELKR
or
an antigen binding fragment or an equivalent
of thereof.

In some embodiments, the light chain variable region comprises a CDRL1 sequence comprising, or alternatively consisting essentially of, or yet further consisting of, an amino acid sequence beginning with RASQSISNNLH (SEQ ID NO: 7), KSSQSLLNSGNQKNYM (SEQ ID NO: 8), or an equivalent each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL2 sequence comprising, or alternatively consisting essentially of, or yet further consisting of, an amino acid sequence beginning with YASQSIS (SEQ ID NO: 9), GASTRES (SEQ ID NO: 10), or an equivalent each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL3 sequence comprising, or alternatively consisting essentially of, or yet further consisting of, an amino acid sequence beginning with QQSNTWPYT (SEQ ID NO: 11), QNDHSYPLT (SEQ ID NO: 12), or an equivalent each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In another aspect of the present disclosure, the antigen binding domain of a FLT3 antibody includes one or more of the following characteristics:
(a) the light chain immunoglobulin variable domain sequence comprises one or more CDRs that are at least 80% identical to a CDR of a light chain variable domain of any of the disclosed light chain sequences;
(b) the heavy chain immunoglobulin variable domain sequence comprises one or more CDRs that are at least 80% identical to a CDR of a heavy chain variable domain of any of the disclosed heavy chain sequences;
(c) the light chain immunoglobulin variable domain sequence is at least 80% identical to a light chain variable domain of any of the disclosed light chain sequences;
(d) the HC immunoglobulin variable domain sequence is at least 80% identical to a heavy chain variable domain of any of the disclosed light chain sequences; and
(e) the antibody binds an epitope that overlaps with an epitope bound by any of the disclosed sequences.

Additional examples of equivalents include peptide having at least 85%, or alternatively at least 90%, or alternatively at least 95%, or alternatively at least 97% amino acid identity to the peptide or a polypeptide that is encoded by a polynucleotide that hybridizes under conditions of high stringency to the complement of a polynucleotide encoding the antigen binding domain, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Exemplary antigen binding domains can comprise one or more of the below noted peptides, and in one aspect may comprise the all three CDRs of the noted HC and LC for a particular antigen disclosed in Table 1 or the polynucleotides encoding the FLT3 HC and LC variable regions, provided below Table 1.

TABLE 1

| ANTI- | | | | | | |
|---|---|---|---|---|---|---|
| FLT3 | | | | | | |
| ANTIBODY | CDRH1 | CDRH2 | CDRH3 | CDRL1 | CDRL2 | CDRL3 |
| FLT3-1 | SYWMH | EIDPSDSYKD | AITTTPFDF | RASQSISNNL | YASQSIS | QQSNT |
| | (SEQ | YNQKFKD | (SEQ ID | H (SEQ ID | (SEQ ID | WPYT |
| | ID NO: | (SEQ ID | NO: 5) | NO: 7) | NO: 9) | (SEQ ID |
| | 1) | NO: 3) | | | | NO: 11) |
| FLT3-2 | NYGLH | VIWSGGSTDY | GGIYYANH | KSSQSLLNSG | GASTRES | QNDHS |
| | (SEQ | NAAFIS (SEQ | YYAMDY | NQKNYM | (SEQ ID | YPLT |
| | ID NO: | ID NO: 4) | (SEQ ID | (SEQ ID | NO: 10) | (SEQ ID |
| | 2) | | NO: 6) | NO: 8) | | NO: 12) |

FLT3-1 Heavy Chain Variable Region polynucleotide sequence:
(SEQ ID NO: 36)
CAGGTCCAACTGCAGCAGCCTGGGGCTGAGCTTGTGAAGCCTGGGGCTTCATTGA
AGCTGTCCTGCAAGTCTTCCGGGTACACCTTCACCAGCTACTGGATGCACTGGGT
GAGGCAGAGGCCTGGACATGGCCTTGAGTGGATCGGAGAGATTGATCCTTCTGA
CAGTTATAAAGACTACAATCAGAAGTTCAAGGACAAGGCCACATTGACTGTGGA
CAGATCCTCCAACACAGCCTACATGCACCTCAGCAGCCTGACATCTGATGACTCT
GCGGTCTATTATTGTGCAAGAGCGATTACGACGACCCCCTTTGACTTCTGGGGCC
AAGGCACCACTCTCACAGTCTCCTCA,
or
an equivalent thereof.
FLT3-1 Light Chain Variable Region polynucleotide sequence:
(SEQ ID NO: 37)
GATATTGTGCTAACTCAGTCTCCAGCCACCCTGTCTGTGACTCCAGGAGATAGCG
TCAGTCTTTCCTGCAGGGCCAGCCAGAGTATTAGCAACAACCTACACTGGTATCA
ACAAAAATCACATGAGTCTCCAAGGCTTCTCATCAAGTATGCTTCCCAGTCCATC
TCTGGGATCCCCTCCAGGTTCAGTGGCAGTGGATCAGGGACAGATTTCACTCTCA
GTATCAACAGTGTGGAGACTGAAGATTTTGGAGTGTATTTCTGTCAACAGAGTAA
CACCTGGCCGTACACGTTCGGAGGGGGGACCAAGCTGGAAATAAAACGG,
or
an equivalent thereof.
FLT3-2 Heavy Chain Variable Region polynucleotide sequence:
(SEQ ID NO: 38)
CAGGTGCAGCTGAAGCAGTCAGGACCTGGCCTAGTGCAGCCCTCACAGAGCCTG
TCCATCACCTGCACAGTCTCTGGTTTCTCATTAACTAACTATGGTTTACACTGGGT
TCGCCAGTCTCCAGGAAAGGGCCTGGAGTGGCTGGGAGTGATATGGAGTGGTGG
AAGCACAGACTATAATGCAGCTTTCATATCCAGACTGAGCATCAGCAAGGACAA
CTCCAAGAGCCAAGTTTTCTTTAAAATGAACAGTCTGCAGGCTGATGACACAGCC
ATATACTACTGTGCCAGAAAAGGAGGGATCTACTATGCTAACCATTACTATGCTA
TGGACTACTGGGGTCAAGGAACCTCAGTCACCGTCTCCTCA,
or
an equivalent thereof.
FLT3-2 Light Chain Variable Region polynucleotide sequence:
(SEQ ID NO: 39)
GACATTGTGATGACACAGTCTCCATCCTCCCTGAGTGTGTCAGCAGGAGAGAAG
GTCACTATGAGCTGCAAGTCCAGTCAGAGTCTGTTAAACAGTGGAAATCAAAAG
AACTATATGGCCTGGTATCAGCAGAAACCAGGGCAGCCTCCTAAACTGTTGATCT
ACGGGGCATCCACTAGGGAATCTGGGGTCCCTGATCGCTTCACAGGCAGTGGAT
CTGGAACCGATTTCACTCTTACCATCAGCAGTGTGCAGGCTGAAGACCTGGCAGT
TTATTACTGTCAGAATGATCATAGTTATCCGCTCACGTTCGGTGCTGGGACCAAG
CTGGAGCTGAAACGG,
or
an equivalent thereof.

Further non-limiting examples of FLT3 CDR domain amino acid sequences are described in Tables 1-4 of the US Patent Application No.: US20180346601, Table V of US Patent Application No.: US20180037657, Table 10 of US Patent Application No.: US20170037149, Table V of US Patent Application No.: US20160272716, Tables 1-3 of US Patent Application No.: US20110091470 and Tables 1-3 of US Patent Application No.: US20090297529.

Non-limiting examples of FLT3 heavy chain variable region and light chain variable region amino acid sequences are described in Tables 1 and 3 of the US Patent Application No.: US20180346601, Table X of US Patent Application No.: US20180037657, Table 10 of US Patent Application No.: US20170037149 and Table VII of US Patent Application No.: US20160272716.

In one aspect, the present disclosure provides the antigen binding domain of an antibody that is at least 80%, or alternatively 85%, or alternatively 90%, or alternatively 95%, or alternatively at least 97%, identical to an FLT3-1. Additional examples of equivalents include polypeptide that is encoded by a polynucleotide that hybridizes under conditions of high stringency to the complement of a polynucleotide encoding the nucleic acid sequence of the antigen binding domain, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some aspects of the antibodies provided herein, the HC variable domain sequence comprises a variable domain sequence of FLT3-1 and the LC variable domain sequence comprises a variable domain sequence of FLT3-1.

In one aspect, the present disclosure provides the antigen binding domain of an antibody comprising the CDRs of FLT3-1. In one aspect, the present disclosure provides the antigen binding domain of antibody that is at least 85%, or alternatively 80%, or alternatively 85%, or alternatively 90%, or alternatively 95%, or alternatively at least 97% identical to the CDRs of FLT3-1, or a polypeptide that is encoded by a polynucleotide that hybridizes under conditions of high stringency to the complement of a polynucleotide encoding the nucleic acid sequence of the CDRs of FLT3, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In one aspect, the present disclosure provides the antigen binding domain of an antibody that is at least 80%, or alternatively 85%, or alternatively 90%, or alternatively 95%, or alternatively at least 97%, identical to an FLT3-2. Additional examples of equivalents include polypeptide that is encoded by a polynucleotide that hybridizes under conditions of high stringency to the complement of a polynucleotide encoding the nucleic acid sequence of the antigen binding domain, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some aspects of the antibodies provided herein, the HC variable domain sequence comprises a variable domain sequence of FLT3-2 and the LC variable domain sequence comprises a variable domain sequence of FLT3-2.

In one aspect, the present disclosure provides the antigen binding domain of an antibody comprising the CDRs of FLT3-2. In one aspect, the present disclosure provides the antigen binding domain of antibody that is at least 85%, or alternatively 80%, or alternatively 85%, or alternatively 90%, or alternatively 95%, or alternatively at least 97% identical to the CDRs of FLT3-2, or a polypeptide that is encoded by a polynucleotide that hybridizes under conditions of high stringency to the complement of a polynucleotide encoding the nucleic acid sequence of the CDRs of FLT3, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Transmembrane Domain. The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain. The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this disclosure may be derived from FcR, TCR, CD3, CDS, CD22, CD79a, CD79b, CD66d. In some embodiments, the signaling domain of the CAR can comprise a CD3 ζ signaling domain.

Since signals generated through the TCR are alone insufficient for full activation of a T-cell, a secondary or co-stimulatory signal may also be required. Thus, the intracellular region of a co-stimulatory signaling molecule, including but not limited the intracellular domains of the proteins CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, may also be included in the cytoplasmic domain of the CAR. For instance, a CAR may comprise one, two, or more co-stimulatory domains, in addition to a signaling domain (e.g., a CD3 ζ signaling domain).

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, OX40, CD30, CD40, PD-1, ICOS, LFA-1, CD2, CD7, CD27, LIGHT, NKG2C, B7-H3, and CD3-zeta protein.

In specific embodiments, the CAR comprises, or alternatively consists essentially thereof, or yet consists of an antigen binding domain of an FLT3 antibody (e.g., an scFv), a hinge domain, a CD28 transmembrane domain, a costimulatory signaling region, and a CD3 zeta signaling domain. In further embodiments, the costimulatory signaling region comprises either or both a CD28 costimulatory signaling region and a 4-1BB costimulatory signaling region.

Switch Mechanisms. In some embodiments, the CAR may also comprise a switch mechanism for controlling expression and/or activation of the CAR. For example, a CAR may comprise, consist, or consist essentially of an extracellular, transmembrane, and intracellular domain, in which the extracellular domain comprises a target-specific binding element that binds a label, binding domain, or tag that is specific for a molecule other than the target antigen that is expressed on or by a target cell. In such embodiments, the specificity of the CAR is provided by a second construct that comprises, consists, or consists essentially of a target antigen binding domain (e.g., an anti-FLT3 antibody or antigen binding fragment thereof or a bispecific antibody that binds FLT3 and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, the cell that expresses the CAR can be administered to a subject, but it cannot bind its target antigen (i.e., FLT3) until the second composition comprising an FLT3-specific binding domain is administered.

CARs of the present disclosure may likewise require multimerization in order to active their function (see, e.g., US 2015/0368342, US 2016/0175359, US 2015/0368360) and/or an exogenous signal, such as a small molecule drug (US 2016/0166613, Yung et al., Science, 2015) in order to elicit a T-cell response.

Furthermore, the disclosed CARs can comprise a “suicide switch” (also referred to as a “suicide gene”) to induce cell death of the CAR cells following treatment (Buddee et al., PLoS One, 2013) or to downregulate expression of the CAR following binding to the target antigen (WO 2016/011210). A non-limiting exemplary suicide switch or suicide gene is iCasp. The suicide switch may be under the direction of an inducible promoter.

In one aspect, the CAR of this disclosure may further can comprise, or alternatively consist essentially of, or yet further consist of an inducible or a constitutively active element. In one embodiment, the inducible or the constitutively active element controls the expression of a polynucleotide encoding an immunoregulatory molecule or a cytokine. The immunoregulatory molecule or cytokine can comprise, or alternatively consist essentially of, or yet further consist of one or more of B7.1, CCL19, CCL21, CD40L, CD137L, GITRL, GM-CSF, IL-12, IL-2, low-toxicity IL-2, IL-15, IL-18, IL-21, LEC, and/or OX40L. In another aspect, the immunoregulatory molecule or cytokine can comprise, or alternatively consist essentially of, or yet further consist of IL-12 and/or GM-CSF; and/or IL-12 and/or one or more of IL-2 and low-toxicity IL-2; and/or IL-12 and/or IL-15; and/or IL-12 and/or IL-21; IL-12 and/or B7.1; and/or IL-12 and/or OX40L; and/or IL-12 and/or CD40L; and/or IL-12 and/or GITRL; and/or IL-12 and/or IL-18; and/or one or more of IL-2 and low-toxicity IL-2 and one or more of CCL19, CCL21, and LEC; and/or IL-15 and one or more of CCL19, CCL21, and LEC; and/or IL-21 and one or more of CCL19, CCL21, and LEC; and/or GM-CSF and one or more of CCL19, CCL21, and LEC; and/or OX40L and one or more of CCL19, CCL21, and LEC; and/or CD137L and one or more of CCL19, CCL21, and LEC; and/or comprises B7.1 and one or more of CCL19, CCL21, and LEC; and/or CD40L and one or more of CCL19, CCL21, and LEC; and/or GITRL and one or more of CCL19, CCL21, and LEC.

In some embodiments, the CAR can further comprise a detectable marker or purification marker. In another aspect, the CARs as described herein are contained in a composition, e.g., a pharmaceutically acceptable carrier for diagnosis or therapy.

### Antibodies and Process for Preparing FLT3, PD-1, and PD-L1 Antibodies

Further provided herein are antibodies comprising, or alternatively consisting essentially of, or yet further consisting of a single chain variable fragment sequence (scFv) comprising, or alternatively consisting essentially of, or yet further consisting of an amino acid sequence (Q V Q L V Q S G V E V K K P G A S V K V S C K A S G Y T F T N Y Y M Y W V R Q A P G Q G L E W M G G I N P S N G G T N F N E K F K N R V T L T T D S S T T T A Y M E L K S L Q F D D T A V Y Y C A R R D Y R F D M G F D Y W G Q G T T V T V S S G G G G S G G G G S G G G G S D I Q M T Q S P S S L S A S V G D R V T I T C R A S Q D V S T A V A W Y Q Q K P G K A P K L L I Y S A S F L Y S G V P S R F S G S G S G T D F T L T I S S L Q P E D F A T Y Y C Q Q Y L Y H P A T F G Q G T K V E I K R (SEQ ID NO: 13)) or an equivalent thereof. In one aspect, the antibody comprising, or alternatively consisting essentially of, or yet further consisting of a single chain variable fragment sequence (scFv) is encoded by the nucleotide sequence comprising, or alternatively consisting essentially of, or yet further consisting of a nucleic acid sequence:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
or
an equivalent thereof.

In one aspect the antigen binding domain has a binding affinity to PD-1 which is at least about 10⁶, 10⁷, 10⁸, or 10⁹ fold greater than its binding affinity for a molecule unrelated to the PD-1. Also described herein are antibodies comprising, or alternatively consisting essentially of, or yet further consisting of a single chain variable fragment sequence (scFv) comprising, or alternatively consisting essentially of, or yet further consisting of an amino acid sequence:

(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15)),
or
an equivalent thereof.

In one aspect, the antibody comprises, or alternatively consists essentially of, or yet further consists of a single chain variable fragment sequence (scFv) encoded by the nucleotide sequence comprising, or alternatively consisting essentially of, or yet further consisting of a nucleic acid sequence:

(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16)),
or
an equivalent thereof.

In one aspect, the antigen binding domain has a binding affinity to PD-L1 which is at least about 10⁶, 10⁷, 10⁸, or 10⁹ fold greater than its binding affinity for a molecule unrelated to the PD-L1.

Also described herein is a bispecific antibody comprising, or alternatively consisting essentially of, or yet further consisting of a single chain variable fragment sequence (scFv) comprising, or alternatively consisting essentially of, or yet further consisting of an amino acid sequence

(QVQLVQSGVEVKKPGASVKVSCKASGYTFTNYY
MYWVRQAPGQGLEWMGGINPSNGGTNFNEKFKNRVTLTTD
SSTTTAYMELKSLQFDDTAVYYCARRDYRFDMGFDYWGQG
TTVTVSSGGGGSGGGGSGGGGSDIQMTQSPSSLSASVGDRV
TITCRASQDVSTAVAWYQQKPGKAPKLLIYSASFLYSGVPSR
FSGSGSGTDFTLTISSLQPEDFATYYCQQYLYHPATFGQGTK
VEIKR (SEQ ID NO: 13))
and/or,
(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15)),
or
an equivalent of each thereof.

In one aspect, the bispecific antibody comprises, or alternatively consists essentially of, or yet further consists of a single chain variable fragment sequence (scFv) encoded by the nucleotide sequence comprising, or alternatively consisting essentially of, or yet further consisting of a nucleic acid sequence:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
and/or,
(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent of each thereof.

In one aspect, the antigen binding domain has a binding affinity to PD-1 and/or PD-L1 which is at least about 10⁶, 10⁷, 10⁸, or 10⁹ fold greater than its binding affinity for a molecule unrelated to the PD-1 and/or PD-L1. The antibody can be an IgA, an IgD, an IgE, an IgG or an IgM antibody. In one particular aspect, the antibody comprises, or alternatively consists essentially of, or yet further consists of a constant region. The constant region may comprise, or alternatively consist essentially of, or yet further consist of an IgA, an IgD, an IgE, an IgG or an IgM constant region. In some embodiments, the constant region is an IgGI constant region or an Ig kappa constant region. In certain embodiments, the constant regions comprise, or alternatively consist essentially of, or yet further consist of the amino acid sequences provided below:

Human IgD constant region, Uniprot: P01880
(SEQ ID NO: 69)
APTKAPDVFPIISGCRHPKDNSPVVLACLITGYHPTSVTVTWYMGTQSQ
PQRTFPEIQRRDSYYMTSSQLSTPLQQWRQGEYKCVVQHTASKSKKEIF
RWPESPKAQASSVPTAQPQAEGSLAKATTAPATTRNTGRGGEEKKKEKE
KEEQEERETKTPECPSHTQPLGVYLLTPAVQDLWLRDKATFTCFVVGSD
LKDAHLTWEVAGKVPTGGVEEGLLERHSNGSQSQHSRLTLPRSLWNAGT
SVTCTLNHPSLPPQRLMALREPAAQAPVKLSLNLLASSDPPEAASWLLC
EVSGFSPPNILLMWLEDQREVNTSGFAPARPPPQPGSTTFWAWSVLRVP
APPSPQPATYTCVVSHEDSRTLLNASRSLEVSYVTDHGPMK
Human IgG1 constant region, Uniprot: P01857
(SEQ ID NO: 70)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Human IgG2 constant region, Uniprot: P01859
(SEQ ID NO: 71)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKS
RWQQGNVFSCSVMHEALHNHYTQKSLSLSPGK
Human IgG3 constant region, Uniprot: P01860
(SEQ ID NO: 72)
ASTKGPSVFPLAPCSRSTSGGTAALGCLVKDYFPEPVTVSWNSGALTSG
VHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEP
KSCDTPPPCPRCPAPELLGGPSVFLFPPKPKDTLMISRTPEVTCVVVDV
SHEDPEVQFKWYVDGVEVHNAKTKPREEQYNSTFRVVSVLTVLHQDWLN
GKEYKCKVSNKALPAPIEKTISKTKGQPREPQVYTLPPSREEMTKNQVS
LTCLVKGFYPSDIAVEWESSGQPENNYNTTPPMLDSDGSFFLYSKLTVD
KSRWQQGNIFSCSVMHEALHNRFTQKSLSLSPGK
Human IgM constant region, Uniprot: P01871
(SEQ ID NO: 73)
GSASAPTLFPLVSCENSPSDTSSVAVGCLAQDFLPDSITLSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLGQSMFT
CRVDHRGLTFQQNASSMCVPDQDTAIRVFAIPPSFASIFLTKSTKLTCL
VTDLTTYDSVTISWTRONGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVSEEEWNTGETYTCVAHEALPNRVTERTVDKSTGKPTLYNVS
LVMSDTAGTCY
Human IgG4 constant region, Uniprot: P01861
(SEQ ID NO: 74)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
Human IgA1 constant region, Uniprot: P01876
(SEQ ID NO: 75)
ASPTSPKVFPLSLCSTQPDGNVVIACLVQGFFPQEPLSVTWSESGQGVT
ARNFPPSQDASGDLYTTSSQLTLPATQCLAGKSVTCHVKHYTNPSQDVT
VPCPVPSTPPTPSPSTPPTPSPSCCHPRLSLHRPALEDLLLGSEANLTC
TLTGLRDASGVTFTWTPSSGKSAVQGPPERDLCGCYSVSSVLPGCAEPW
NHGKTFTCTAAYPESKTPLTATLSKSGNTFRPEVHLLPPPSEELALNEL
VTLTCLARGFSPKDVLVRWLQGSQELPREKYLTWASRQEPSQGTTTFAV
TSILRVAAEDWKKGDTFSCMVGHEALPLAFTQKTIDRLAGKPTHVNVSV
VMAEVDGTCY
Human IgA2 constant region, Uniprot: P01877
(SEQ ID NO: 76)
ASPTSPKVFPLSLDSTPQDGNVVVACLVQGFFPQEPLSVTWSESGQNVT
ARNFPPSQDASGDLYTTSSQLTLPATQCPDGKSVTCHVKHYTNPSQDVT
VPCPVPPPPPCCHPRLSLHRPALEDLLLGSEANLTCTLTGLRDASGATF
TWTPSSGKSAVQGPPERDLCGCYSVSSVLPGCAQPWNHGETFTCTAAHP
ELKTPLTANITKSGNTFRPEVHLLPPPSEELALNELVTLTCLARGFSPK
DVLVRWLQGSQELPREKYLTWASRQEPSQGTTTFAVTSILRVAAEDWKK
GDTFSCMVGHEALPLAFTQKTIDRMAGKPTHVNVSVVMAEVDGTCY
Human Ig kappa constant region, Uniprot: P01834
(SEQ ID NO: 77)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

This disclosure also relates to antibodies that competes for binding with the antibodies described herein. The antibody of this disclosure may be a polyclonal, a monoclonal or a humanized antibody. Also provided herein are the antigen binding fragments of the antibodies of this disclosure. The antigen binding fragment may be selected from the group consisting of Fab, F(ab′)2, Fab′, scFv, and Fv. In one aspect, the antigen binding fragment may comprise, or alternatively consist essentially of, or yet further consist of an amino acid sequence (Q V Q L V Q S G V E V K K P G A S V K V S C K A S G Y T F T N Y Y M Y W V R Q A P G Q G L E W M G G I N P S N G G T N F N E K F K N R V T L T T D S S T T T A Y M E L K S L Q F D D T A V Y Y C A R R D Y R F D M G F D Y W G Q G T T V T V S S G G G G S G G G G S G G G G S D I Q M T Q S P S S L S A S V G D R V T I T C R A S Q D V S T A V A W Y Q Q K P G K A P K L L I Y S A S F L Y S G V P S R F S G S G S G T D F T L T I S S L Q P E D F A T Y Y C Q Q Y L Y H P A T F G Q G T K V E I K R (SEQ ID NO: 13)), or an equivalent thereof. The antigen binding fragment can be encoded by the nucleotide sequence comprising, or alternatively consisting essentially of, or yet further consisting of a nucleic acid sequence:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14)),
or
an equivalent of each thereof.

In another aspect, the antigen binding fragment may comprise, or alternatively consist essentially of, or yet further consist of an amino acid sequence:

(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15)),
or
an equivalent of each thereof.

The antigen binding fragment can be encoded by the nucleotide sequence comprising, or alternatively consisting essentially of, or yet further consisting of a nucleic acid sequence:

(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16))
or
an equivalent of each thereof.

Also described herein are polypeptides comprising, or alternatively consisting essentially of, or yet further consisting of an amino acid sequence of any one of:

45 (QVQLVQSGVEVKK
PGASVKVSCKASGYTFTNYYMYWVRQAPGQGLEWMGGINP
SNGGTNFNEKFKNRVTLTTDSSTTTAYMELKSLQFDDTAVY
YCARRDYRFDMGFDYWGQGTTVTVSSGGGGSGGGGSGGGG
SDIQMTQSPSSLSASVGDRVTITCRASQDVSTAVAWYQQKP
GKAPKLLIYSASFLYSGVPSRFSGSGSGTDFTLTISSLQPEDF
ATYYCQQYLYHPATFGQGTKVEIKR (SEQ ID NO: 13))
or
(EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCA
RRHWPGGFDYWGQGTLVTVSAGGGGSGGGGSGGGGSEIVLTQSPATLSL
SPGERATLSCRASKGVSTSGYSYLHWYQQKPGEAPRLLIYLASYLESGV
PARFSGSGSGTDFTLTISSLEPEDFAVYYCQHSRDLPLTFGGGTKVEIK
(SEQ ID NO: 15)),
or
an equivalent of each thereof.

This disclosure further relates to isolated nucleic acids comprising, or alternatively consisting essentially of, or yet further consisting of a nucleic acid sequence of any one of:

(CAGGTCCAATTGGTACAGAGCGGCGTCGAAGTAAAGAAGCCTGGAGCC
AGCGTTAAAGTTTCTTGCAAGGCTTCAGGATATACTTTCACTAACTACT
ATATGTACTGGGTACGGCAGGCTCCAGGGCAAGGGTTGGAGTGGATGGG
AGGGATCAATCCTTCTAACGGCGGCACTAACTTTAACGAAAAATTTAAA
AATAGGGTGACCCTCACAACTGACTCAAGTACGACTACAGCATACATGG
AACTCAAATCTCTCCAATTCGATGACACGGCTGTCTATTATTGCGCGAG
AAGAGACTATCGCTTCGATATGGGGTTTGATTATTGGGGGCAAGGTACT
ACGGTTACCGTCAGCTCCGGGGGTGGCGGCTCCGGCGGCGGTGGGTCAG
GTGGAGGAGGGTCTGACATTCAGATGACGCAATCCCCAAGCTCTCTGTC
CGCGTCAGTGGGCGACCGAGTTACAATCACATGCCGCGCTTCTCAAGAT
GTGTCAACCGCTGTCGCCTGGTACCAACAGAAGCCTGGGAAGGCCCCTA
AGCTTCTCATCTACTCAGCTTCTTTTCTGTACTCAGGGGTACCGTCTAG
ATTCTCAGGATCCGGTAGTGGGACGGACTTCACATTGACCATAAGTTCC
TTGCAGCCTGAGGATTTCGCTACATATTATTGCCAACAGTACCTTTACC
ATCCTGCCACTTTTGGCCAGGGTACTAAGGTCGAGATCAAACGG
(SEQ ID NO: 14))
or
(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16)),
or
an equivalent of each thereof.

Also provided herein are isolated cells comprising, or alternatively consisting essentially of, or consisting the antibodies of this disclosure. In one aspect, the antibodies of this disclosure are expressed in isolated cells. The cell can be a prokaryotic or a eukaryotic cell, and is optionally selected from an animal cell, a mammalian cell, a bovine cell, a feline cell, a canine cell, a murine cell, an equine cell or a human cell. In some embodiments, the eukaryotic cell is an immune cell, optionally a T-cell, a B-cell, a NK-cell, a dendritic cell, a myeloid cell, a monocyte, or a macrophage. In further embodiments, the immune cell is a T-cell, which may be optionally modified to suppress endogenous TCR expression, using any appropriate system, e.g., a CRISPR system. In any of the above embodiments relating to an isolated cell, the isolated cell expresses the CAR on the cell surface and secretes the antibody comprising an antigen binding domain that recognizes and binds PD-1 and/or PD-L1 or antigen binding fragment thereof, optionally the bispecific antibody.

Antibodies for use in this disclosure can be purchased or prepared using methods known in the art and briefly described herein. In some aspect, it may be desirable to produce an antibody that is specific to an antigen expressed by the target cell that has been isolated from the patient for specialized, personalized therapy. Their manufacture and uses are well known and disclosed in, for example, Greenfield (2014) Antibodies: A Laboratory Manual, Cold Spring Harbor Laboratory Press, Cold Spring Harbor, N.Y. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as a C-terminal fragment FLT3, PD-1, or PD-L1 or an isolated polypeptide. Depending on the host species, various adjuvants may be added and used to increase an immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain aspects, the antibodies of the present disclosure are polyclonal, i.e., a mixture of plural types of FLT3, PD-1, or PD-L1 antibodies having different amino acid sequences. In one aspect, the polyclonal antibody comprises a mixture of plural types of FLT3, PD-1, or PD-L1 antibodies having different CDRs. As such, a mixture of cells which produce different antibodies is cultured, and an antibody purified from the resulting culture can be used (see WO 2004/061104).

Monoclonal Antibody Production. Monoclonal antibodies to FLT3, PD-1, or PD-L1 antigen may be prepared using any technique which provides for the production of antibody molecules by continuous cell lines in culture and in one aspect, are generated to specially bind an antigen isolated from the subject to be treated. Such techniques include, but are not limited to, the hybridoma technique (see, e.g., Kohler & Milstein (1975) Nature 256: 495-497); the trioma technique; the human B-cell hybridoma technique (see, e.g., Kozbor et al. (1983) Immunol. Today 4: 72) and the EBV hybridoma technique to produce human monoclonal antibodies (see, e.g., Cole et al. (1985) in: MONOCLONAL ANTIBODIES AND CANCER THERAPY, Alan R. Liss, Inc., pp. 77-96). Human monoclonal antibodies can be utilized in the practice of the present technology and can be produced by using human hybridomas (see, e.g., Cote et al. (1983) Proc. Natl. Acad. Sci. 80: 2026-2030) or by transforming human B-cells with Epstein Barr Virus in vitro (see, e.g., Cole et al. (1985) in: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or antigen binding fragment thereof for an antigen or epitope present on the FLT3, PD-1, or PD-L1 antigen polypeptide. Alternatively, hybridomas expressing FLT3 monoclonal antibodies can be prepared by immunizing a subject, e.g., with an isolated polypeptide comprising, or alternatively consisting essentially of, or yet further consisting of, the amino acid sequence of the FLT3, PD-1, or PD-L1 antigen or a fragment thereof, and then isolating hybridomas from the subject's spleen using routine methods. See, e.g., Milstein et al., (Galfre and Milstein (1981) Methods Enzymol 73:3-46). Screening the hybridomas using standard methods will produce monoclonal antibodies of varying specificity (i.e., for different epitopes) and affinity. A selected monoclonal antibody with the desired properties, e.g., FLT3 PD-1, or PD-L1 antigen binding, can be (i) used as expressed by the hybridoma, (ii) bound to a molecule such as polyethylene glycol (PEG) to alter its properties, or (iii) a cDNA encoding the nucleic acid sequence of the monoclonal antibody can be isolated, sequenced and manipulated in various ways. In one aspect, the FLT3 monoclonal antibody is produced by a hybridoma which includes a B-cell obtained from a transgenic non-human animal, e.g., a transgenic mouse, having a genome comprising a human heavy chain transgene and a light chain transgene fused to an immortalized cell. Hybridoma techniques include those known in the art and taught in Greenfield (2014) Antibodies: A Laboratory Manual Cold Spring Harbor Laboratory, Cold Spring Harbor, N.Y.; Hammerling et al. (1981) Monoclonal Antibodies And T-Cell Hybridomas:563-681.

Phage Display Technique. As noted above, the antibodies of the present disclosure can be produced through the application of recombinant DNA and phage display technology. For example, FLT3, PD-1, or PD-L1 antibodies, can be prepared using various phage display methods known in the art. In phage display methods, functional antibody domains are displayed on the surface of a phage particle which carries polynucleotide sequences encoding them. Phage with a desired binding property is selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse et al. (1989) Science 246:1275-1281) to allow rapid and effective identification of monoclonal Fab fragments with the desired specificity for FLT3 polypeptide, e.g., a polypeptide or derivatives, fragments, analogs or homologs thereof. Other examples of phage display methods that can be used to make the isolated antibodies of the present disclosure include those disclosed in Huston et al. (1988) Proc. Natl. Acad. Sci. U.S.A. 85:5879-5883; Chaudhary et al. (1990) Proc. Natl. Acad. Sci. U.S.A., 87:1066-1070; Brinkman et al. (1995) J. Immunol. Methods 182:41-50; Ames et al. (1995) J. Immunol. Methods 184:177-186; Kettleborough et al. (1994) Eur. J. Immunol. 24:952-958; Persic et al. (1997) Gene 187: 9-18; Burton et al. (1994) Advances in Immunology 57:191-280; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al. (1992) BioTechniques 12:864-869; Sawai et al. (1995) AJRI 34:26-34; and Better et al. (1988) Science 240:1041-1043.

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See, e.g., Barbas III et al. (2001) Phage Display, A Laboratory Manual (Cold Spring Harbor Laboratory Press, Cold Spring Harbor, N.Y.). However, other vector formats could be used for this process, such as cloning the antibody fragment library into a lytic phage vector (modified T7 or Lambda Zap systems) for selection and/or screening.

Alternate Methods of Antibody Production. Antibodies may also be produced by inducing in vivo production in the lymphocyte population or by screening recombinant immunoglobulin libraries or panels of highly specific binding reagents (Orlandi et al. (1989) PNAS 86:3833-3837; Winter, G. et al. (1991) Nature 349:293-299).

Alternatively, techniques for the production of single chain antibodies may be used. Single chain antibodies (scFvs) comprise a heavy chain variable region and a light chain variable region connected with a linker peptide (typically around 5 to 25 amino acids in length). In the scFv, the variable regions of the heavy chain and the light chain may be derived from the same antibody or different antibodies. scFvs may be synthesized using recombinant techniques, for example by expression of a vector encoding the nucleic acid sequence of the scFv in a host organism such as E. coli. DNA encoding the nucleic acid sequence of the scFv can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding the nucleic acid sequence of the scFv and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments.

Alternatively, Fab expression libraries may be constructed to allow rapid and easy identification of monoclonal Fab fragments with the desired specificity (Huse et al., Science, 256: 1275-1281 (1989)).

Commercially Available Antibodies. Antibodies may also be purchased from commercially available sources. Examples of commercially available FLT3 antibodies include, but are not limited to, those produced by suppliers such as Proteintech Group Inc., eBioscience, Abgent, Aviva Systems Biology, Becton Dickinson (Biosciences), Cell Signaling Technology, Fitzgerald Industries International, United States Biological, Biorbyt, Abbexa, Abgent, LifeSpan BioSciences, antibodies-online, Rockland Immunochemicals, Inc., OriGene Technologies, GeneTex, Raybiotech, Inc., Acris Antibodies GmbH, Sino Biological, MyBioSource.com, Bioss Inc., St. John's Laboratory, Source BioScicne, Abcam, ProSci, Inc., Clinic Sciences, Novus Biologicals, Creative Diagnostics, Thermo Scientific Pierce Antibodies, PeproTech, MBL International, Miltenyi Biotec, GenWay Biotech, Inc., LifeSpan Biosciences, Bioworld Technology, EXBIO Praha, a.s., Novus Biologicals, BioVision, Bethyl Laboratories, Santa Crus Biotechnology Inc., AbD Serotec, BioRad, BioLegend, Thermo Fisher Scientific, EMD Milipore, R&D Systems, Cell Sciences, Progen Biotechnik GmbH, Spring Bioscience, Atlas Antibodies, Abbiotec, Bostrebio, Nordic BioSite, and other commonly known antibody manufacturers. Non-limiting examples of commercially available FLT3 antibodies include those from BV10 and 4G8 clones and biological equivalents or modified versions thereof, including, but not limited to the following commercially available antibodies listed by supplier and catalog number: antibodies-online ABIN487499, antibodies-online ABIN487500, LifeSpan Biosciences LS-C179623-100, LifeSpan Biosciences LS-C179624-50, Acris Antibodies AM20042AF-N, Acris Antibodies AM20042FC-N, MBL International K0107-3, MBL International K0107-4, Novus Biologicals NBP1-54522-0.05 mg, Novus Biologicals NBP1-54414, Santa Cruz Biotechnology, Inc. sc-21788, Becton Dickinson Biosciences 564708, Becton Dickinson Biosciences 563494. Further exemplary commercially available antibodies include all antibodies listed as reactive to human FLT3 on Biocompare or another database of commercially available antibodies; non-limiting examples include those disclosed herein, listed by supplier and catalog number Proteintech Group Inc. 21049-1-AP, Proteintech Group Inc. 15827-1-AP, Proteintech Group Inc. 15826-1-AP, eBioscience 17-1357-41, eBioscience 12-1357-41, eBioscience 14-1357-80, eBioscience 17-1357-42, eBioscience 12-1357-42, eBioscience 14-1357-82, Abgent AP7644a, Abgent AP3068a, Aviva Systems Biology OAAB17159, Aviva Systems Biology OAAF00442, Aviva Systems Biology ARP30009_T100, Aviva Systems Biology ARP30010_P050, Cell Signaling Technology 3462S, Cell Signaling Technology 3464S, Cell Signaling Technology 3474S, Cell Signaling Technology 3466S, Cell Signaling Technology 3461S, Cell Signaling Technology 3461L, Cell Signaling Technology 3463S, Cell Signaling Technology 4577S, Fitzgerald Industries International 20R-2351, Fitzgerald Industries International 70R-12259, Fitzgerald Industries International 70R-17325. Commercially available antibodies for PD-1 and PD-L1 are available. See, e.g., biocompare.com/pfu/110447/soids/531283/Antibodies/PD1 (describing commercial sources of anti-PD-1 antibodies; last accessed on Jul. 3, 2019) and biocompare.com/pfu/110447/soids/592604/Antibodies/PDL1 (describing commercial sources of anti-PD-L1 antibodies; last accessed on Jul. 3, 2019). One of skill in the art can detect expression of FLT3, PD-1 an/or PD-L1 using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis, ELISA (enzyme-linked immunosorbent assay) or other immunoassays etc.

Antibody Equivalents. The present disclosure provides for “equivalents” or “biological equivalents” of the above disclosed antibodies, wherein an antigen binding domain of an antibody that is at least 80%, or alternatively 85%, or alternatively 90%, or alternatively 95%, or alternatively at least 97%, identical to the antigen binding domain of any of the above disclosed antibodies renders it the above disclosed antibody's biological equivalent. Additional examples of equivalents include polypeptide that is encoded by a polynucleotide that hybridizes under conditions of high stringency to the complement of a polynucleotide encoding the nucleic acid sequence of the antigen binding domain of any one of the above disclosed antibodies, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Antibody Modifications. The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFv molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

Antibody Screening. Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the FLT3, PD-1, or PD-L1 antigen, or any fragment or oligopeptide thereof and its specific antibody. A two-site, monoclonal-based immunoassay utilizing monoclonal antibodies specific to two non-interfering FLT3, PD-1, or PD-L1 antigen epitopes may be used, but a competitive binding assay may also be employed (Maddox et al. (1983) J. Exp. Med. 158:1211-1216).

Antibody Purification. The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

### Isolated Nucleic Acids and Processes for Preparing CARs

Further aspects relate to an isolated nucleic acid comprising, or alternatively consists essentially of, or yet further consists a sequence comprising, or alternatively consisting essentially of, or yet further consisting of, an antigen binding domain of an FLT3 antibody; a hinge domain; a transmembrane domain—for example, a CD28 transmembrane domain; one or more costimulatory regions—for example, selected from a CD28 costimulatory signaling region, a 4-1BB costimulatory signaling region, an ICOS costimulatory signaling region, and an OX40 costimulatory region; and a CD3 zeta signaling domain.

Not to be bound by theory, further aspects contemplate an isolated nucleic acid comprises, or alternatively consists essentially of, or yet further consists of, the binding domain of a ligand for an exogenous molecule (i.e. not FLT3); a hinge domain; a transmembrane domain—for example, a CD28 transmembrane domain; one or more costimulatory regions—for example, selected from a CD28 costimulatory signaling region, a 4-1BB costimulatory signaling region, an ICOS costimulatory signaling region, and an OX40 costimulatory region; and a CD3 zeta signaling domain. In further such aspects, the binding domain of a ligand for an exogenous molecule recognizes and binds an antigen binding domain of an FLT3 antibody operatively linked to the exogenous molecule; thus, generating a FLT3 CAR.

In some embodiments, the isolated nucleic acid further comprises, or alternatively consists essentially of, or yet further consists of a polynucleotide sequence encoding the nucleic acid sequence of an antibody or antigen binding fragment thereof, which optionally recognizes and binds PD-1 and/or PD-L1. In other embodiments, a second isolated nucleic acid is provided comprising, or alternatively consisting essentially of, or yet further consisting of a polynucleotide sequence encoding the nucleic acid sequence of an antibody or antigen binding fragment thereof, which optionally recognizes and binds PD-1 and/or PD-L1. In either of these embodiments, the antibody or antigen binding fragment thereof may comprise, or alternatively consist essentially of, or further consist of a PD-1 antagonist or agonist and/or a PD-L1 antagonist or agonist. In certain embodiments, the antibody or antigen binding fragment thereof comprises, or alternatively consists essentially thereof, or further consists of the relevant CDR regions of an antibody to PD-1 and/or an antibody to PD-L1, or an equivalent of each thereof. In some embodiments, the antibody or antigen binding fragment thereof comprises, or alternatively consists essentially thereof, or further consists of the heavy chain and/or light chain variable region of an antibody to PD-1 and/or PD-L1, and/or an equivalent of each thereof. In some embodiments, the antibody or antigen binding fragment thereof comprises a single chain variable fragment (scFv) comprising an antigen binding domain of a PD-1 antibody and/or a single chain variable fragment (scFv) comprising an antigen binding domain of a PD-L1 antibody, and/or an equivalent each thereof. In some embodiments, the scFv comprises an amino acid sequence encoded by the polynucleotide sequence:

Anti-PD-1 antibody scFv polynucleotide sequence:
(SEQ ID NO: 14)
CAGGTCCAATTGGTACAGAGCGGCGTCGAAGTAAAGAAGCCTGGAGCCA
GCGTTAAAGTTTCTTGCAAGGCTTCAGGATATACTTTCACTAACTACTA
TATGTACTGGGTACGGCAGGCTCCAGGGCAAGGGTTGGAGTGGATGGGA
GGGATCAATCCTTCTAACGGCGGCACTAACTTTAACGAAAAATTTAAAA
ATAGGGTGACCCTCACAACTGACTCAAGTACGACTACAGCATACATGGA
ACTCAAATCTCTCCAATTCGATGACACGGCTGTCTATTATTGCGCGAGA
AGAGACTATCGCTTCGATATGGGGTTTGATTATTGGGGGCAAGGTACTA
CGGTTACCGTCAGCTCCGGGGGTGGCGGCTCCGGCGGCGGTGGGTCAGG
TGGAGGAGGGTCTGACATTCAGATGACGCAATCCCCAAGCTCTCTGTCC
GCGTCAGTGGGCGACCGAGTTACAATCACATGCCGCGCTTCTCAAGATG
TGTCAACCGCTGTCGCCTGGTACCAACAGAAGCCTGGGAAGGCCCCTAA
GCTTCTCATCTACTCAGCTTCTTTTCTGTACTCAGGGGTACCGTCTAGA
TTCTCAGGATCCGGTAGTGGGACGGACTTCACATTGACCATAAGTTCCT
TGCAGCCTGAGGATTTCGCTACATATTATTGCCAACAGTACCTTTACCA
TCCTGCCACTTTTGGCCAGGGTACTAAGGTCGAGATCAAACGG,
or
an equivalent thereof.
Anti-PD-1 antibody scFv amino acid sequence:
(SEQ ID NO: 13)
QVQLVQSGVEVKKPGASVKVSCKASGYTFTNYYMYWVRQA
PGQGLEWMGGINPSNGGTNFNEKFKNRVTLTTDSSTTTAYM
ELKSLQFDDTAVYYCARRDYRFDMGFDYWGQGTTVTVSSG
GGGSGGGGSGGGGSDIQMTQSPSSLSASVGDRVTITCRASQ
DVSTAVAWYQQKPGKAPKLLIYSASFLYSGVPSRFSGSGSG
TDFTLTISSLQPEDFATYYCQQYLYHPATFGQGTKVEIKR,
or
an equivalent thereof.

In some embodiments, the antibody or antigen binding fragment thereof comprises a single chain variable fragment (scFv) comprising an antigen binding domain of a PD-L1 antibody. In some embodiments, the scFv comprises an amino acid sequence encoded by the polynucleotide sequence:

Anti-PD-L1 antibody scFv polynucleotide sequence:
(SEQ ID NO: 16)
GAAGTTCAGTTGGTCGAGTCAGGAGGAGGCCTGGTGCAACCCGGGGGCT
CACTCCGGTTGTCCTGTGCTGCTTCAGGATTTACGTTTTCTGACTCATG
GATACATTGGGTGCGCCAAGCCCCGGGCAAGGGGCTGGAATGGGTGGCC
TGGATCTCTCCGTATGGGGGTTCCACCTACTATGCTGATTCAGTAAAAG
GACGGTTCACTATAAGCGCGGATACAAGTAAGAATACTGCCTATCTTCA
AATGAATTCTCTTCGCGCCGAGGATACAGCGGTATATTATTGCGCTAGA
CGACATTGGCCAGGGGGCTTTGACTATTGGGGGCAGGGTACTCTTGTGA
CCGTTAGTGCGGGAGGTGGTGGCAGCGGTGGAGGCGGCTCCGGGGGTGG
TGGTTCAGAAATTGTCCTGACTCAATCCCCTGCCACATTGAGTTTGAGC
CCAGGAGAGAGAGCAACTCTGTCATGCCGGGCGTCAAAAGGTGTCAGTA
CGTCAGGCTACTCCTATCTTCATTGGTATCAGCAGAAACCGGGAGAAGC
GCCGCGCCTTCTCATATACCTGGCTAGTTACCTTGAGAGTGGCGTCCCG
GCCCGGTTTAGTGGGAGTGGGTCTGGGACTGATTTTACGCTGACAATCA
GCAGTCTTGAGCCAGAGGACTTCGCGGTTTACTATTGCCAACATTCACG
CGATTTGCCCCTCACCTTCGGCGGTGGAACGAAGGTTGAAATAAAA,
or
an equivalent thereof.
Anti-PD-L1 antibody scFv amino acid sequence:
(SEQ ID NO: 15)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSAGGGGSGGGGSGGGGSEIVLTQSPATLSLS
PGERATLSCRASKGVSTSGYSYLHWYQQKPGEAPRLLIYLASYLESGVP
ARFSGSGSGTDFTLTISSLEPEDFAVYYCQHSRDLPLTFGGGTKVEIK,
or
an equivalent thereof.

In some embodiments, the antibody or antigen binding fragment thereof is a bispecific antibody. In certain embodiments, the bispecific antibody comprises, or alternatively consists essentially thereof, or further consists of the relevant CDR regions of an antibody to PD-1 and/or an antibody to PD-L1, or an equivalent of each thereof. In certain embodiments, the bispecific antibody comprises, or alternatively consists essentially thereof, or further consists of the relevant CDR regions of an antibody to PD-1 and/or an antibody to PD-L1, or an equivalent of each thereof. In some embodiments, the bispecific antibody comprises, or alternatively consists essentially thereof, or further consists of the heavy chain and/or light chain variable region of an antibody to PD-1 and/or PD-L1, and/or an equivalent of each thereof. In some embodiments, the bispecific antibody comprises a single chain variable fragment (scFv) comprising an antigen binding domain of a PD-1 antibody and/or a single chain variable fragment (scFv) comprising an antigen binding domain of a PD-L1 antibody, and/or an equivalent each thereof.

Provided herein is an isolated nucleic acid or a vector comprising, or alternatively consisting essentially of, or yet further consisting of: a polynucleotide encoding a chimeric antigen receptor (CAR) comprising, or alternatively consisting essentially of, or yet further consisting of: (a) an antigen binding domain of an FLT3 antibody; (b) a hinge domain; (c) a transmembrane domain; (d) and an intracellular domain; and a polynucleotide encoding an antibody or antigen binding fragment thereof comprising, or alternatively consisting essentially of, or yet further consisting of an antigen binding domain that recognizes and binds PD-1 and/or PD-L1.

The isolated nucleic acid or vector disclosed above encoding the CAR can comprise, or alternatively consist essentially of, or yet further consist of any CAR disclosed herein. In one aspect, the isolated nucleic acid or the vector of this disclosure encoding the CAR further comprises, or alternatively consists essentially of, or yet further consists of a signaling domain. In another aspect, the isolated nucleic acid or the vector encoding the CAR can further comprise, or alternatively consist essentially of, or yet further consist of an inducible or a constitutively active element. In one embodiment, the inducible or the constitutively active element controls the expression of a polynucleotide encoding an immunoregulatory molecule or a cytokine. The immunoregulatory molecule or cytokine can comprise, or alternatively consist essentially of, or yet further consist of one or more of B7.1, CCL19, CCL21, CD40L, CD137L, GITRL, GM-CSF, IL-12, IL-2, low-toxicity IL-2, IL-15, IL-18, IL-21, LEC, and/or OX40L. In another aspect, the immunoregulatory molecule or cytokine can comprise, or alternatively consist essentially of, or yet further consist of IL-12 and/or GM-CSF; and/or IL-12 and/or one or more of IL-2 and low-toxicity IL-2; and/or IL-12 and/or IL-15; and/or IL-12 and/or IL-21; IL-12 and/or B7.1; and/or IL-12 and/or OX40L; and/or IL-12 and/or CD40L; and/or IL-12 and/or GITRL; and/or IL-12 and/or IL-18; and/or one or more of IL-2 and low-toxicity IL-2 and one or more of CCL19, CCL21, and LEC; and/or IL-15 and one or more of CCL19, CCL21, and LEC; and/or IL-21 and one or more of CCL19, CCL21, and LEC; and/or GM-CSF and one or more of CCL19, CCL21, and LEC; and/or OX40L and one or more of CCL19, CCL21, and LEC; and/or CD137L and one or more of CCL19, CCL21, and LEC; and/or comprises B7.1 and one or more of CCL19, CCL21, and LEC; and/or CD40L and one or more of CCL19, CCL21, and LEC; and/or GITRL and one or more of CCL19, CCL21, and LEC.

In one embodiment, the hinge domain of the isolated nucleic acid or the vector encoding the CAR comprises, or alternatively consists essentially of, or yet further consists of a CD8 α hinge domain. In another aspect, the transmembrane domain of the isolated nucleic acid or the vector encoding the CAR comprises, or alternatively consists essentially of, or yet further consists of a CD8a transmembrane domain. In a separate aspect, the costimulatory signaling region of the isolated nucleic acid or the vector encoding the CAR comprises, or alternatively consists essentially of, or yet further consists of a CD28 costimulatory signaling region and/or a 4-1BB costimulatory signaling region

In some embodiments, the isolated nucleic acid or the vector encoding the CAR comprises, or alternatively consists essentially of, or yet further consists of: (a) an antigen binding domain of a FLT3 antibody; (b) a CD8 α hinge domain; (c) a CD8 α transmembrane domain; and (d) a CD28 costimulatory signaling region and/or a 4-1BB costimulatory signaling region. In other embodiments, the isolated nucleic acid or the vector encoding the CAR comprises, or alternatively consists essentially of, or yet further consists of: (a) an antigen binding domain of a FLT3 antibody; (b) a CD8 α hinge domain; (c) a CD8 α transmembrane domain; (d) a CD28 costimulatory signaling region and/or a 4-1BB costimulatory signaling region; and (e) a CD3 zeta signaling domain.

For any of the isolated nucleic acid or vector disclosed above, the antigen binding domain of the FLT3 antibody of the isolated nucleic acid or the vector encoding the CAR can comprise, or alternatively consist essentially of, or yet further consist of a heavy chain variable region comprising, or alternatively consisting essentially of, or yet further consisting of:

a CDHR1 having the amino acid sequence
(SYWMH (SEQ ID NO: 1))
or
(NYGLH (SEQ ID NO: 2))
or
an equivalent of each thereof,
a CDHR2 having the amino acid sequence
(EIDPSDSYKDYNQKFKD (SEQ ID NO: 3))
or
(VIWSGGSTDYNAAFIS (SEQ ID NO: 4))
or
an equivalent of each thereof,
and
a CDHR3 having the amino acid sequence encoded by
(AITTTPFDF (SEQ ID NO: 5))
or
(GGIYYANHYYAMDY (SEQ ID NO: 6))
or
an equivalent of each thereof,
and/or
a light chain variable region comprising:
a CDLR1 having the amino acid sequence
(RASQSISNNLH (SEQ ID NO: 7))
or
(KSSQSLLNSGNQKNYM (SEQ ID NO: 8))
or
an equivalent of each thereof,
a CDLR2 having the amino acid sequence
(YASQSIS (SEQ ID NO: 9))
or
(GASTRES (SEQ ID NO: 10))
or
an equivalent of each thereof,
and
a CDLR3 having the amino acid sequence
(QQSNTWPYT (SEQ ID NO: 11))
or
(QNDHSYPLT (SEQ ID NO: 12))
or
an equivalent of each thereof.

For any of the isolated nucleic acid or vector disclosed above, the antigen binding domain of the isolated nucleic acid or vector encoding the antibody or antigen binding fragment thereof that recognizes and binds PD-1 and/or PD-L1 may comprise, or alternatively consist essentially of, or yet further consist of a PD-1 antagonist and/or a PD-L1 antagonist, and/or an equivalent each thereof. In one aspect, the antigen binding domain or antigen binding fragment that recognizes and binds PD-1 and/or PD-L1 comprises, or alternatively consists essentially of, or yet further consists of CDR regions of an antibody to PD-1 and/or PD-L1, and/or an equivalent of each thereof. In another aspect, the antibody or antigen binding fragment that recognizes and binds PD-1 and/or PD-L1 comprises, or alternatively consists essentially of, or yet further consists of a heavy chain and a light chain variable region of an antibody to PD-1 and/or PD-L1, and/or an equivalent of each thereof. In a further aspect, the antibody or antigen binding fragment that recognizes and binds PD-1 and/or PD-L1 comprises, or alternatively consists essentially of, or yet further consists of a single chain variable fragment (scFv) comprising, or alternatively consisting essentially of, or yet further consisting of an antigen binding domain of a PD-1 antibody and/or a single chain variable fragment (scFv) comprising, or alternatively consisting essentially of, or yet further consisting of an antigen binding domain of a PD-L1 antibody, and/or an equivalent each thereof.

In one embodiment, the antibody that recognizes and binds PD-1 and/or PD-L1 is a bispecific antibody. In another embodiment, the bispecific antibody thereof comprises, or alternatively consists essentially of, or yet further consists of a PD-1 antagonist and a PD-L1 antagonist, and, optionally, further comprises, or alternatively consists essentially of, or yet further consists of a linker. In a further embodiment, the bispecific antibody thereof comprises, or alternatively consists essentially of, or yet further consists of CDR regions of an antibody to PD-1 and PD-L1, and may optionally, further comprise, or alternatively consist essentially of, or yet further consist of a linker. In one aspect, the bispecific antibody comprises, or alternatively consists essentially of, or yet further consists of a heavy chain and light chain variable region of an antibody to PD-1 and PD-L1 and, may optionally, further comprise, or alternatively consist essentially of, or yet further consist of a linker. In another embodiment, the bispecific antibody comprises, or alternatively consists essentially of, or yet further consists of a single chain variable fragment (scFv) comprising, or alternatively consisting essentially of, or yet further consisting of an antigen binding domain of a PD-1 antibody and a single chain variable fragment (scFv) comprising, or alternatively consisting essentially of, or yet further consisting of an antigen binding domain of a PD-L1 antibody and, may optionally, further comprise, or alternatively consist essentially of, or yet further consist of a linker.

In one particular embodiment, single chain variable fragment (scFv) comprising, or alternatively consisting essentially of, or yet further consisting of an antigen binding domain of a PD-L1 antibody comprises, or alternatively consists essentially of, or yet further consists of the polynucleotide sequence:

(GAAGTTCAGTTGGTCGAGTCAGGAGGAGGCCTGGTGCAACCCGGGGGC
TCACTCCGGTTGTCCTGTGCTGCTTCAGGATTTACGTTTTCTGACTCAT
GGATACATTGGGTGCGCCAAGCCCCGGGCAAGGGGCTGGAATGGGTGGC
CTGGATCTCTCCGTATGGGGGTTCCACCTACTATGCTGATTCAGTAAAA
GGACGGTTCACTATAAGCGCGGATACAAGTAAGAATACTGCCTATCTTC
AAATGAATTCTCTTCGCGCCGAGGATACAGCGGTATATTATTGCGCTAG
ACGACATTGGCCAGGGGGCTTTGACTATTGGGGGCAGGGTACTCTTGTG
ACCGTTAGTGCGGGAGGTGGTGGCAGCGGTGGAGGCGGCTCCGGGGGTG
GTGGTTCAGAAATTGTCCTGACTCAATCCCCTGCCACATTGAGTTTGAG
CCCAGGAGAGAGAGCAACTCTGTCATGCCGGGCGTCAAAAGGTGTCAGT
ACGTCAGGCTACTCCTATCTTCATTGGTATCAGCAGAAACCGGGAGAAG
CGCCGCGCCTTCTCATATACCTGGCTAGTTACCTTGAGAGTGGCGTCCC
GGCCCGGTTTAGTGGGAGTGGGTCTGGGACTGATTTTACGCTGACAATC
AGCAGTCTTGAGCCAGAGGACTTCGCGGTTTACTATTGCCAACATTCAC
GCGATTTGCCCCTCACCTTCGGCGGTGGAACGAAGGTTGAAATAAAA
(SEQ ID NO: 16)),
or
an equivalent thereof.

In yet another embodiment, single chain variable fragment (scFv) comprising, or alternatively consisting essentially of, or yet further consisting of an antigen binding domain of a PD-1 antibody comprises, or alternatively consists essentially of, or yet further consists of the polynucleotide sequence:

(SEQ ID NO: 14)
(CAGGTCCAATTGGTACAGAGCGGCGTCGAAGTAAAGAAGCCTGGAGCCA
GCGTTAAAGTTTCTTGCAAGGCTTCAGGATATACTTTCACTAACTACTAT
ATGTACTGGGTACGGCAGGCTCCAGGGCAAGGGTTGGAGTGGATGGGAGG
GATCAATCCTTCTAACGGCGGCACTAACTTTAACGAAAAATTTAAAAATA
GGGTGACCCTCACAACTGACTCAAGTACGACTACAGCATACATGGAACTC
AAATCTCTCCAATTCGATGACACGGCTGTCTATTATTGCGCGAGAAGAGA
CTATCGCTTCGATATGGGGTTTGATTATTGGGGGCAAGGTACTACGGTTA
CCGTCAGCTCCGGGGGTGGCGGCTCCGGCGGCGGTGGGTCAGGTGGAGGA
GGGTCTGACATTCAGATGACGCAATCCCCAAGCTCTCTGTCCGCGTCAGT
GGGCGACCGAGTTACAATCACATGCCGCGCTTCTCAAGATGTGTCAACCG
CTGTCGCCTGGTACCAACAGAAGCCTGGGAAGGCCCCTAAGCTTCTCATC
TACTCAGCTTCTTTTCTGTACTCAGGGGTACCGTCTAGATTCTCAGGATC
CGGTAGTGGGACGGACTTCACATTGACCATAAGTTCCTTGCAGCCTGAGG
ATTTCGCTACATATTATTGCCAACAGTACCTTTACCATCCTGCCACTTTT
GGCCAGGGTACTAAGGTCGAGATCAAACGG, or an equivalent
thereof.

In one aspect, the vector described herein is a plasmid. In another aspect, the vector is a viral vector selected from a retroviral vector, a lentiviral vector, an adenoviral vector, or an adeno-associated viral vector. In a further aspect, the vector is bicistronic.

The isolated nucleic acid or vector of this disclosure can further comprise, or alternatively consist essentially of, or yet further consist of a promoter and/or enhancer operatively linked to the polynucleotide encoding the antibody or the antigen binding fragment that recognizes and binds PD-1 and/or PD-L1. In some embodiments, the promoter and/or enhancer operatively linked to the polynucleotide encoding the antibody or the antigen binding fragment that recognizes and binds PD-1 and/or PD-L1 is a high expression promoter. Non-limiting examples of high expression promoters are the cytomegalovirus (CMV), myeloproliferative sarcoma virus enhancer (MND) and EF1 alpha promoters.

In certain embodiments, methods of producing FLT3 CAR expressing cells are disclosed, the method comprising, or alternatively consisting essentially of or yet further consisting of transducing a population of isolated cells with a nucleic acid sequence encoding a FLT3 CAR and a nucleic acid sequence encoding an antibody or antigen binding fragment thereof—optionally, a bispecific antibody—which, optionally, recognizes and binds PD-1 and/or PD-L1 an equivalent of each thereof. In one aspect, the method of producing the CAR expressing cell comprises, or alternatively consists essentially of or yet further consists of transducing the isolated cell with the isolated nucleic acid or the vector of this disclosure. The isolated cells can be selected from a group consisting of T-cells, B-cells, NK-cells, dendritic cells, myeloid cells, monocytes, or macrophages. In some embodiments, this is achieved through (i) the use of a vector encoding the FLT3 CAR construct and the antibody or antigen binding fragment thereof or (ii) the use of two vectors, one encoding the FLT3 CAR and the other encoding the antibody or antigen binding fragment thereof. In some embodiments, this is achieved through the use of mRNA encoding the FLT3 CAR construct and/or the antibody or antigen binding fragment thereof, which in turn may be introduced into cells via electroporation. See, e.g., Choi et al. (2010) Biomed Microdevices 12(5):855-863. In a further aspect, a subpopulation of cells that have been successfully transduced with said nucleic acid sequence is selected. In some embodiments, the isolated cells are T-cells, an animal T-cell, a mammalian T-cell, a feline T-cell, a canine T-cell or a human T-cell, thereby producing FLT3 CAR T-cells. In certain embodiments, the isolated cell is an NK-cell, e.g., an animal NK-cell, a mammalian NK-cell, a feline NK-cell, a canine NK-cell or a human NK-cell, thereby producing FLT3 CAR NK-cells. In some embodiments, the isolated cells are B-cells, an animal B-cell, a mammalian B-cell, a feline B-cell, a canine B-cell or a human B-cell, thereby producing FLT3 CAR B-cells.

Not to be bound by theory, further aspects contemplate methods of transduction of a cell with an isolated nucleic acid comprising, or alternatively consisting essentially of, or yet further consisting of a sequence comprising, or alternatively consisting essentially of, or yet further consisting of, the binding domain of a ligand for an exogenous molecule (i.e. not FLT3); a hinge domain; a transmembrane domain—for example, a CD28 transmembrane domain; one or more costimulatory regions—for example, selected from a CD28 costimulatory signaling region, a 4-1BB costimulatory signaling region, an ICOS costimulatory signaling region, and an OX40 costimulatory region; and a CD3 zeta signaling domain, i.e. a “universal CAR cell.” In further such aspects, the binding domain of a ligand for an exogenous molecule that recognizes and the exogenous molecule, which is operatively linked to an antigen binding domain of an FLT3 antibody operatively linked; and thus, an FLT3 CAR is generated upon introduction of the antigen binding domain of an FLT3 antibody operatively linked to the exogenous molecule. In some embodiments, the exogenous molecule is biotin or streptavidin.

In some embodiments, T-cells expressing the disclosed CARs may be further modified to reduce or eliminate expression of endogenous TCRs. Reduction or elimination of endogenous TCRs can reduce off-target effects and increase the effectiveness of the T-cells. T-cells stably lacking expression of a functional TCR may be produced using a variety of approaches. T-cells internalize, sort, and degrade the entire T-cell receptor as a complex, with a half-life of about 10 hours in resting T-cells and 3 hours in stimulated T-cells (von Essen, M. et al. (2004) J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T-cell, which all must signal properly. Thus, if a TCR complex is destabilized with proteins that do not associate properly or cannot signal optimally, the T-cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T-cells. By blocking expression of one or more of these proteins, the T-cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T-cell having a stable deficiency in functional TCR expression.

Expression of inhibitory RNAs (e.g., shRNA, siRNA, miRNA, etc.) in primary T-cells can be achieved using any conventional expression system, e.g., a lentiviral expression system. Although lentiviruses are useful for targeting resting primary T-cells, not all T-cells will express the shRNAs. Some of these T-cells may not express sufficient amounts of the RNAs to allow enough inhibition of TCR expression to alter the functional activity of the T-cell. Thus, T-cells that retain moderate to high TCR expression after viral transduction can be removed, e.g., by cell sorting or separation techniques, so that the remaining T-cells are deficient in cell surface TCR or CD3, enabling the expansion of an isolated population of T-cells deficient in expression of functional TCR or CD3.

Expression of CRISPR in primary T-cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T-cells or other suitable immune cells for CAR cell therapy. Further, to the extent that CRISPR editing is unsuccessful, cells can be selected for success according to the methods disclosed above. For example, as noted above, T-cells that retain moderate to high TCR expression after viral transduction can be removed, e.g., by cell sorting or separation techniques, so that the remaining T-cells are deficient in cell surface TCR or CD3, enabling the expansion of an isolated population of T-cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Sources of Isolated Cells. Prior to expansion and genetic modification of the cells disclosed herein, cells may be obtained from a subject—for instance, in embodiments involving autologous therapy—or a commercially available cell culture, e.g., American Type Culture Collection (ATCC).

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies Dynabeads® system; STEMcell Technologies EasySep™, RoboSep™ RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells.

Alternatively, cells may be obtained through commercially available cell cultures, including but not limited to, for T-cells, lines BCL2 (AAA) Jurkat (ATCC® CRL-2902™) BCL2 (S70A) Jurkat (ATCC® CRL-2900™), BCL2 (S87A) Jurkat (ATCC® CRL-2901™) BCL2 Jurkat (ATCC® CRL-2899™), Neo Jurkat (ATCC® CRL-2898™); for B-cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75](ATCC® CRL-2625™), DS-1 (ATCC® CRL-11102™), EB-3 [EB3](ATCC® CCL-85™) Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK-cells, lines NK-92 (ATCC® CRL-2407™), NK-92MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); cutaneous T-cell lymphoma lines, e.g., HuT78 (ATCC CRM-TIB-161), MJ[G11](ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK-YS, NOI-90, and YT. Null leukemia cell lines, including but not limited to REH, NALL-1, KM-3, L92-221, are other commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

Vectors. CAR cells may be prepared using vectors comprising the polynucleotides as described above. Thus, the present disclosure provides: (i) a vector, optionally a bicistronic vector, comprising a polynucleotide sequence encoding the nucleic acid sequence of a FLT3 CAR or a complement or equivalent thereof and, optionally, further comprising an a polynucleotide sequence encoding the nucleic acid sequence of an antibody or an antigen binding fragment thereof, which optionally recognizes and binds PD-1 and/or PD-L1 an equivalent of each thereof or (ii) a vector comprising an a polynucleotide sequence encoding the nucleic acid sequence of a FLT3 CAR or a complement or equivalent thereof and a vector comprising an a polynucleotide sequence encoding the nucleic acid sequence of an antibody or an antigen binding fragment thereof, which optionally recognizes and binds PD-1 and/or PD-L1 an equivalent of each thereof. In some embodiments, the antibody or antigen binding fragment thereof comprises, or alternatively consists essentially of, or yet further consists of a PD-1 antagonist or agonist and/or a PD-L1 antagonist or agonist an equivalent of each thereof. In certain embodiments, the antibody or antigen binding fragment thereof comprises, or alternatively consists essentially thereof, or yet further consists of the relevant CDR regions of an antibody to PD-1 and/or an antibody to PD-L1, or an equivalent of each thereof. In some embodiments, the antibody or antigen binding fragment thereof comprises, or alternatively consists essentially thereof, or yet further consists of the heavy chain and/or light chain variable region of an antibody that recognizes and binds to PD-1 and/or PD-L1, and/or an equivalent of each thereof. In some embodiments, the antibody or antigen binding fragment thereof comprises, or alternatively consists essentially thereof, or yet further consists of, a single chain variable fragment (scFv) comprising an antigen binding domain of a PD-1 antibody and/or a single chain variable fragment (scFv) comprising an antigen binding domain of a PD-L1 antibody, and/or an equivalent each thereof. In some embodiments, the antibody or antigen binding fragment thereof comprises, or alternatively consists essentially of, or yet further consists of, a single chain variable fragment (scFv) comprising an antigen binding domain of a PD-1 antibody, that comprises an amino acid sequence encoded by:

Anti-PD-1 scFv:
(SEQ ID NO: 14)
CAGGTCCAATTGGTACAGAGCGGCGTCGAAGTAAAGAAGCCTGGAGCCAG
CGTTAAAGTTTCTTGCAAGGCTTCAGGATATACTTTCACTAACTACTATA
TGTACTGGGTACGGCAGGCTCCAGGGCAAGGGTTGGAGTGGATGGGAGGG
ATCAATCCTTCTAACGGCGGCACTAACTTTAACGAAAAATTTAAAAATAG
GGTGACCCTCACAACTGACTCAAGTACGACTACAGCATACATGGAACTCA
AATCTCTCCAATTCGATGACACGGCTGTCTATTATTGCGCGAGAAGAGAC
TATCGCTTCGATATGGGGTTTGATTATTGGGGGCAAGGTACTACGGTTAC
CGTCAGCTCCGGGGGTGGCGGCTCCGGCGGCGGTGGGTCAGGTGGAGGAG
GGTCTGACATTCAGATGACGCAATCCCCAAGCTCTCTGTCCGCGTCAGTG
GGCGACCGAGTTACAATCACATGCCGCGCTTCTCAAGATGTGTCAACCGC
TGTCGCCTGGTACCAACAGAAGCCTGGGAAGGCCCCTAAGCTTCTCATCT
ACTCAGCTTCTTTTCTGTACTCAGGGGTACCGTCTAGATTCTCAGGATCC
GGTAGTGGGACGGACTTCACATTGACCATAAGTTCCTTGCAGCCTGAGGA
TTTCGCTACATATTATTGCCAACAGTACCTTTACCATCCTGCCACTTTTG
GCCAGGGTACTAAGGTCGAGATCAAACGG, or an equivalent
thereof.

In some embodiments, the antibody or antigen binding fragment thereof comprises or alternatively consists essentially of, or yet further consists of, a single chain variable fragment (scFv) derived from an antibody to PD-L1 that comprises, or alternatively consists essentially of, or yet further consists of, an amino acid sequence encoded by the polynucleotide sequence:

Anti-PD-L1 scFv:
(SEQ ID NO: 16)
GAAGTTCAGTTGGTCGAGTCAGGAGGAGGCCTGGTGCAACCCGGGGGCTC
ACTCCGGTTGTCCTGTGCTGCTTCAGGATTTACGTTTTCTGACTCATGGA
TACATTGGGTGCGCCAAGCCCCGGGCAAGGGGCTGGAATGGGTGGCCTGG
ATCTCTCCGTATGGGGGTTCCACCTACTATGCTGATTCAGTAAAAGGACG
GTTCACTATAAGCGCGGATACAAGTAAGAATACTGCCTATCTTCAAATGA
ATTCTCTTCGCGCCGAGGATACAGCGGTATATTATTGCGCTAGACGACAT
TGGCCAGGGGGCTTTGACTATTGGGGGCAGGGTACTCTTGTGACCGTTAG
TGCGGGAGGTGGTGGCAGCGGTGGAGGCGGCTCCGGGGGTGGTGGTTCAG
AAATTGTCCTGACTCAATCCCCTGCCACATTGAGTTTGAGCCCAGGAGAG
AGAGCAACTCTGTCATGCCGGGCGTCAAAAGGTGTCAGTACGTCAGGCTA
CTCCTATCTTCATTGGTATCAGCAGAAACCGGGAGAAGCGCCGCGCCTTC
TCATATACCTGGCTAGTTACCTTGAGAGTGGCGTCCCGGCCCGGTTTAGT
GGGAGTGGGTCTGGGACTGATTTTACGCTGACAATCAGCAGTCTTGAGCC
AGAGGACTTCGCGGTTTACTATTGCCAACATTCACGCGATTTGCCCCTCA
CCTTCGGCGGTGGAACGAAGGTTGAAATAAAA, or an equivalent
thereof.

In some embodiments, the antibody or antigen binding fragment thereof is a bispecific antibody. In some embodiments, the bispecific antibody comprises, or alternatively consists essentially of, or yet further consists of, a PD-1 antagonist or agonist and/or a PD-L1 antagonist or agonist. In certain embodiments, the bispecific antibody comprises, or alternatively consists essentially thereof, or yet further consists of the relevant CDR regions of an antibody that recognizes and binds to PD-1 and/or an antibody to PD-L1, or an equivalent of each thereof. In some embodiments, the bispecific antibody comprises, or alternatively consists essentially thereof, or further consists of the heavy chain and/or light chain variable region of an antibody to PD-1 and/or PD-L1, and/or an equivalent of each thereof. In some embodiments, the bispecific antibody comprises or alternatively consists essentially of, or yet further consists of, a single chain variable fragment (scFv) comprising an antigen binding domain of a PD-1 antibody and/or a single chain variable fragment (scFv) comprising an antigen binding domain of a PD-L1 antibody, and/or an equivalent each thereof. In some embodiments, the bispecific antibody comprises or alternatively consists essentially of, or yet further consists of, a single chain variable fragment (scFv) comprising an antigen binding domain of a PD-1 antibody comprises the anti-PD-1 scFv amino acid sequence provided herein above. In some embodiments, the bispecific antibody comprises or alternatively consists essentially of, or yet further consists of, a single chain variable fragment (scFv) derived from an antibody to PD-L1 that comprises or alternatively consists essentially of, or yet further consists of, the anti-PD-L1 scFv amino acid sequence provided herein above.

In any of the above embodiments, the vector or vectors may optionally comprise, or alternatively consist essentially of, or further consist of a detectable label and/or a polynucleotide conferring antibiotic resistance and/or regulatory elements for the transcription and translation for the CAR and the antigen binding domain that recognizes and binds PD-1 and/or PD-L1.

In any of the above embodiments, each of the polynucleotides may be operatively linked to a regulatory polynucleotide, optionally a promoter and/or enhancer. In some embodiments, the polynucleotide encoding an antibody or antigen binding fragment thereof comprising an antigen binding domain that recognizes and binds PD-1 and/or PD-L1 is operatively linked to a promoter and/or enhancer which allows for overexpression of the antibody or antigen binding fragment thereof.

In some embodiments, the isolated nucleic acid sequence of the FLT3 CAR encodes for a CAR comprising, or alternatively consisting essentially of, or yet further consisting of an antigen binding domain of an FLT3 antibody, a hinge domain, a CD28 transmembrane domain, one or more costimulatory regions selected from a CD28 costimulatory signaling region, a 4-1BB costimulatory signaling region, an ICOS costimulatory signaling region, and an OX40 costimulatory region, and a CD3 zeta signaling domain. In one aspect, the antigen binding domain has a binding affinity to FLT3 which is at least about 10⁶, 10⁷, 10⁸, or 10⁹ fold greater than its binding affinity for a molecule unrelated to the FLT3. In specific embodiments, the isolated nucleic acid sequence comprises, or alternatively consisting essentially thereof, or yet further consisting of, sequences encoding (a) an antigen binding domain of an FLT3 antibody followed by (b) a hinge domain, (c) a CD28 transmembrane domain followed by (d) one or more costimulatory regions selected from a CD28 costimulatory signaling region, a 4-1BB costimulatory signaling region, an ICOS costimulatory signaling region, and an OX40 costimulatory region followed by (e) a CD3 zeta signaling domain.

In certain embodiments, the isolated nucleic acid sequence further comprises, or further consists essentially of, or yet further consists of, a polynucleotide promoter sequence located upstream of the polynucleotide encoding the antigen binding domain of the FLT3 antigen binding domain of the FLT3 antibody. In some embodiments, this promoter is a cytomegalovirus (CMV) promoter sequence, a myeloproliferative sarcoma virus enhancer (MND) promoter, or an EF1 alpha promoter. Non-limiting exemplary sequences of said promoters are provided herein.

CMV promoter sequence:
(SEQ ID NO: 78)
TAGTAATCAATTACGGGGTCATTAGTTCATAGCCCATATATGGAGTTCCG
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTACGCCCCCTATTGACG
TCAATGACGGTAAATGGCCCGCCTGGCATTATGCCCAGTACATGACCTTA
TGGGACTTTCCTACTTGGCAGTACATCTACGTATTAGTCATCGCTATTAC
CATGGTGATGCGGTTTTGGCAGTACATCAATGGGCGTGGATAGCGGTTTG
ACTCACGGGGATTTCCAAGTCTCCACCCCATTGACGTCAATGGGAGTTTG
TTTTGGCACCAAAATCAACGGGACTTTCCAAAATGTCGTAACAACTCCGC
CCCATTGACGCAAATGGGCGGTAGGCGTGTACGGTGGGAGGTCTATATAA
GCAGAGCTGGTTTAGTGAACCGTCAG, and optionally, an
equivalent thereof.
CMV promoter sequence:
(SEQ ID NO: 79)
GCAAATGGGCGGTAGGCGTGTACGGTGGGAGGTTTATATAAGCAGAGCTC
GTTTAGTGAACCGTCAGATC, and optionally, an
equivalent thereof.
MND promoter sequence:
(SEQ ID NO: 80)
AACTAACCAATCAGTTCGCTTCTCGCTTCTGTTCGCGCGCTTCTGCTCCC
CGAGCTCTATATAAGCAGAGCTCGTTTAGTGAACCGTCAGATCGCCTGGA
GACGCCATCCACGCTGTTTTGACCTCCATAGAAGACACCGACTCTAGAGG
ATC, and optionally, an equivalent thereof.
EF1 alpha promoter sequence:
(SEQ ID NO: 81)
AAGGATCTGCGATCGCTCCGGTGCCCGTCAGTGGGCAGAGCGCACATCGC
CCACAGTCCCCGAGAAGTTGGGGGGAGGGGTCGGCAATTGAACGGGTGCC
TAGAGAAGGTGGCGCGGGGTAAACTGGGAAAGTGATGTCGTGTACTGGCT
CCGCCTTTTTCCCGAGGGTGGGGGAGAACCGTATATAAGTGCAGTAGTCG
CCGTGAACGTTCTTTTTCGCAACGGGTTTGCCGCCAGAACACAGCTGAAG
CTTCGAGGGGCTCGCATCTCTCCTTCACGCGCCCGCCGCCCTACCTGAGG
CCGCCATCCACGCCGGTTGAGTCGCGTTCTGCCGCCTCCCGCCTGTGGTG
CCTCCTGAACTGCGTCCGCCGTCTAGGTAAGTTTAAAGCTCAGGTCGAGA
CCGGGCCTTTGTCCGGCGCTCCCTTGGAGCCTACCTAGACTCAGCCGGCT
CTCCACGCTTTGCCTGACCCTGCTTGCTCAACTCTACGTCTTTGTTTCGT
TTTCTGTTCTGCGCCGTTACAGATCCAAGCTGTGACCGGCGCCTAC,
and optionally, an equivalent thereof.

In certain embodiments, the isolated nucleic acid sequence further comprises, or further consists essentially of, or yet further consists of, an inducible caspase (“iCasp”) or other “suicide gene” encoding polynucleotide sequence located upstream of the polynucleotide encoding the antigen binding domain of the FLT3 antigen binding domain of the FLT3 antibody; a non-limiting exemplary polynucleotide sequence of said iCasp gene is provided herein:

iCasp sequence:
(SEQ ID NO: 82)
ATGGGAGTGCAGGTGGAAACCATCTCCCCAGGAGACGGGCGCACCTTCCC
CAAGCGCGGCCAGACCTGCGTGGTGCACTACACCGGGATGCTTGAAGATG
GAAAGAAAGTTGATTCCTCCCGGGACAGAAACAAGCCCTTTAAGTTTATG
CTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATG
GTGCCACTGGGCACCCAGGCATCATCCCACCACATGCCACTCTCGTCTTC
GATGTGGAGCTTCTAAAACTGGAATCTGGCGGTGGATCCGGAGTCGACGG
ATTTGGTGATGTCGGTGCTCTTGAGAGTTTGAGGGGAAATGCAGATTTGG
CTTACATCCTGAGCATGGAGCCCTGTGGCCACTGCCTCATTATCAACAAT
GTGAACTTCTGCCGTGAGTCCGGGCTCCGCACCCGCACTGGCTCCAACAT
CGACTGTGAGAAGTTGCGGCGTCGCTTCTCCTCGCTGCATTTCATGGTGG
AGGTGAAGGGCGACCTGACTGCCAAGAAAATGGTGCTGGCTTTGCTGGAG
CTGGCGCAGCAGGACCACGGTGCTCTGGACTGCTGCGTGGTGGTCATTCT
CTCTCACGGCTGTCAGGCCAGCCACCTGCAGTTCCCAGGGGCTGTCTACG
GCACAGATGGATGCCCTGTGTCGGTCGAGAAGATTGTGAACATCTTCAAT
GGGACCAGCTGCCCCAGCCTGGGAGGGAAGCCCAAGCTCTTTTTCATCCA
GGCCTGTGGTGGGGAGCAGAAAGACCATGGGTTTGAGGTGGCCTCCACTT
CCCCTGAAGACGAGTCCCCTGGCAGTAACCCCGAGCCAGATGCCACCCCG
TTCCAGGAAGGTTTGAGGACCTTCGACCAGCTGGACGCCATATCTAGTTT
GCCCACACCCAGTGACATCTTTGTGTCCTACTCTACTTTCCCAGGTTTTG
TTTCCTGGAGGGACCCCAAGAGTGGCTCCTGGTACGTTGAGACCCTGGAC
GACATCTTTGAGCAGTGGGCTCACTCTGAAGACCTGCAGTCCCTCCTGCT
TAGGGTCGCTAATGCTGTTTCGGTGAAAGGGATTTATA,
and optionally, an equivalent thereof.

In some embodiments, the iCasp gene construct comprises portion of a Caspase 9 operatively linked to an FKBP protein domain. Caspase 9, encoded by the CASP9 gene (GenBank Accession No. NM001229), is a non-limiting example of an initiator caspase and plays a role in the mitochondrial apoptotic pathway; a portion thereof is present in the non-limiting exemplary sequence disclosed above. The FKBP protein domain in the above disclosed non-limiting exemplary sequence is optimized to bind an inducing agent, specifically a chemical inducer of dimerization (CID). In the above disclosed sequence, the chemical inducer is AP1903, a synthetic drug that has proven safe in healthy volunteers. It is envisioned that equivalents of both the FKBP domain and the chemical inducer of dimerization (e.g., modified forms of AP1903 or FKBP) may be used in lieu of the listed exemplary embodiments. In some aspect, the dimerization can be induced by any small molecule known to facilitate dimerization of caspase 9. Administration of this small molecule results in cross-linking and activation of Caspase 9, which in turn induces apoptosis of cells expressing the iCasp gene.

iCasp amino acid sequence:
(SEQ ID NO: 83)
MGVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLVF
DVELLKLESGGGSGVDGFGDVGALESLRGNADLAYILSMEPCGHCLIINN
VNFCRESGLRTRTGSNIDCEKLRRRFSSLHFMVEVKGDLTAKKMVLALLE
LAQQDHGALDCCVVVILSHGCQASHLQFPGAVYGTDGCPVSVEKIVNIFN
GTSCPSLGGKPKLFFIQACGGEQKDHGFEVASTSPEDESPGSNPEPDATP
FQEGLRTFDQLDAISSLPTPSDIFVSYSTFPGFVSWRDPKSGSWYVETLD
DIFEQWAHSEDLQSLLLRVANAVSVKGIY, and optionally, an
equivalent thereof.

In certain embodiments, the isolated nucleic acid sequence further comprises, or further consists essentially of, or yet further consists of, a 2A peptide (T2A) encoding polynucleotide sequence located upstream of the polynucleotide encoding the antigen binding domain of the FLT3 antigen binding domain of the FLT3 antibody; a encoding a non-limiting exemplary sequence of said T2A polynucleotide is provided herein:

T2A sequence:
(SEQ ID NO: 84)
GCCGAGGGCAGAGGAAGTCTTCTAACATGCGGTGACGTGGAGGAGAATCC
CGGCCCT, and optionally, an equivalent thereof.
T2A amino acid sequence:
(SEQ ID NO: 85)
AEGRGSLLTCGDVEENPGP,
and optionally an equivalent thereof.

In embodiments involving T2A, T2A-mediated “self-cleavage” may give rise to a 1:1 ratio of the two separate proteins.

In certain embodiments, the isolated nucleic acid sequence further comprises, or further consists essentially of, or yet further consists of, a signal peptide encoding polynucleotide sequence located upstream of the polynucleotide encoding the antigen binding domain of the FLT3 antigen binding domain of the FLT3 antibody; polynucleotides encoding non-limiting exemplary sequences of said signal peptides are provided herein.

Signal Peptide Sequence encoding polynucleotide
sequence:
(SEQ ID NO: 20)
ATGGGATGGAGCTCTATCATCCTCTTCTTGGTAGCAACAGCTACAGGTGT
CCAC, and optionally, an equivalent thereof.
Signal Peptide amino acid sequence:
(SEQ ID NO: 86)
MGWSSIILFLVATATGVH, and optionally an equivalent
thereof.
Signal Peptide Sequence:
(SEQ ID NO: 21)
MGWSCIILFLVATATGVHS, and optionally, an
equivalent thereof.
Signal Peptide Sequence:
(SEQ ID NO: 22)
MDWIWRILFLVGAATGAHS, and optionally, an equivalent
thereof.

In some embodiments, the isolated nucleic acid comprises a detectable label and/or a polynucleotide conferring antibiotic resistance. In one aspect, the label or polynucleotide are useful to select cells successfully transduced with the isolated nucleic acids. In certain embodiments, this detectable label is a protein tag derived from the c-myc gene known as a “myc tag.” A non-limiting exemplary sequence encoding said myc tag is disclosed below.

“myc” sequence:
(SEQ ID NO: 87)
GAGCAGAAGCTGATCAGCGAGGAGGACCTG, and optionally,
an equivalent thereof.
“myc” amino acid sequence:
(SEQ ID NO: 52)
EQKLISEEDL, and optionally, an equivalent
thereof.

In some embodiments, the isolated nucleic acid sequence is comprised within a vector. In certain embodiments, the vector is a plasmid. In other embodiments, the vector is a viral vector. Non-limiting examples of such include without limitation a retroviral vector, a lentiviral vector, an adenoviral vector, and an adeno-associated viral vector. In specific embodiments, the vector is a lentiviral vector.

The preparation of exemplary vectors and the generation of CAR expressing cells using said vectors is discussed in detail in the examples below. In summary, the expression of natural or synthetic nucleic acids encoding CARs or immunoregulatory molecules is typically achieved by operably linking a nucleic acid encoding the CAR polypeptide or portions thereof to a promoter and incorporating the construct into an expression vector. A similar method may be used to construct the isolated nucleic acid sequence comprising a polynucleotide encoding an immunoregulatory molecule. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such include without limitation, human immunodeficiency virus (HIV), equine infectious anemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses and may also comprise synthetic components. Vector components can be manipulated to obtain desired characteristics; such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Further methods of introducing exogenous nucleic acids into the art are known and include but are not limited to gene delivery using one or more of RNA electroporation, nanotechnology, sleeping beauty vectors, retroviruses, and/or adenoviruses. In addition,

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging vector and cell lines. The isolated nucleic acids can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T-cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently cotransfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then cocultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either cotransfection with a selectable marker or infection with pseudotyped virus. In both cases, the vectors integrate. Alternatively, vectors can be introduced in an episomally maintained plasmid. High titer recombinant retrovirus-containing supernatants are produced.

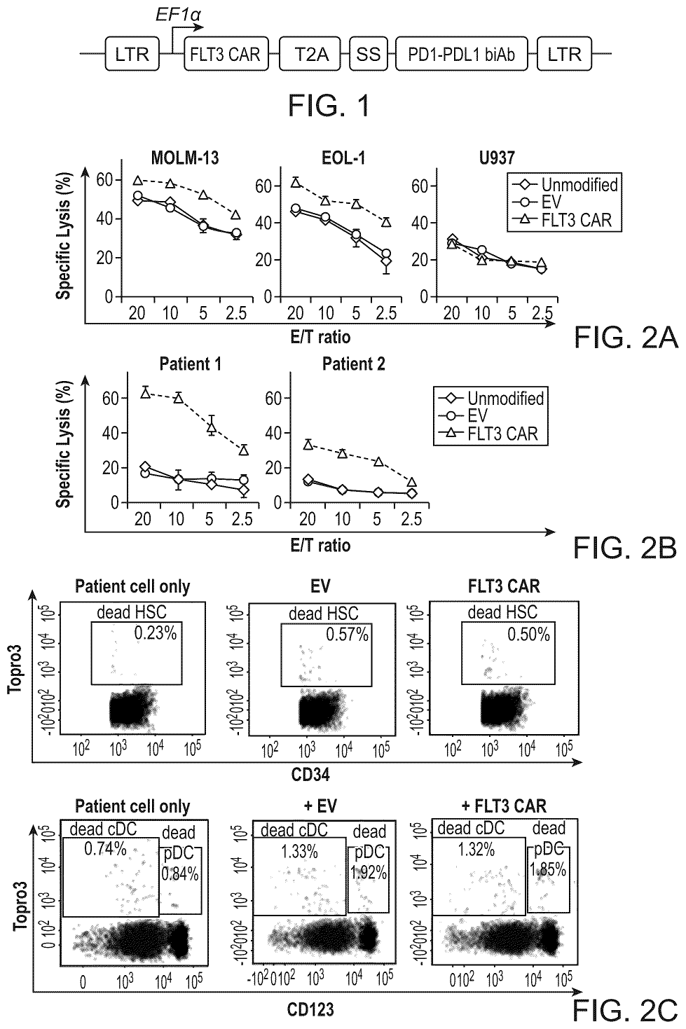

In one specific embodiment, provided herein is an isolated polynucleotide or a vector comprising the elements as set forth in FIG. 1 and equivalents of each of the disclosed elements. FIG. 1 discloses a bicistronic FLT3 CAR with secretory PD-1-PD-L1 bispecific antibody. FLT3 CAR is driven by EF1α promoter. PD-1-PD-L1 biAb is linked with CAR by T2A and led by a secretory signal peptide (SS). The isolated polynucleotide can be inserted into a vector such as a lentiviral vector that is flanked with long terminal repeats.

### FLT3-Specific CAR Cells

Aspects of the present disclosure relate to an isolated cell comprising the isolated polynucleotides and/or vectors as described herein wherein the cell has expressed the polynucleotide. In one aspect, a FLT3 CAR is prepared by expressing the isolated polynucleotides as disclosed herein in a host cell that also expresses or comprises a PD-1 and/or PD-L1 specific antigen binding region. The cell is a prokaryotic or a eukaryotic cell. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell. The eukaryotic cell can be from any preferred species, e.g., an animal cell, a mammalian cell such as a human, a feline or a canine cell. In other embodiments, the eukaryotic cell is an immune cell, optionally a T-cell, a B-cell, a NK-cell, a dendritic cell, a myeloid cell, a monocyte, or a macrophage. The cells are useful therapeutically and diagnostically. In one aspect, the isolated cell of this disclosure expresses the CAR and secretes an antibody, optionally a bispecific antibody.

In specific embodiments, the isolated cell comprises, or alternatively consists essentially of, or yet further consists of an exogenous CAR comprising, or alternatively consisting essentially of, or yet further consisting of, an antigen binding domain of an FLT3 antibody; a hinge domain; a transmembrane domain—for example, a CD28 transmembrane domain; and optionally, one or more costimulatory regions—for example, selected from a CD28 costimulatory signaling region, a 4-1BB costimulatory signaling region, an ICOS costimulatory signaling region, and an OX40 costimulatory region; and a CD3 zeta signaling domain and an antigen binding domain that recognizes and binds PD-1 and/or PD-L1. In a further aspect, the cells have been activated as described below.

Further provided herein are populations of isolated cells of this disclosure. Also provided is a population of the cells that are activated and expanded from a cell described above. The population can be substantially homogeneous, having cell that are at least 50%, or alternatively at least 60%, or alternatively at least 70%, or alternatively at least 75%, or alternatively at least 80%, or alternatively at least 85%, or alternatively at least 90%, or alternatively at least 95%, or alternatively at least 98%, identical.

Activation and Expansion of CAR Cells. Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7, 144,575; 7,067,318; 7,172,869; 7,232,566; 7,175,843; 5,883,223; 6,905,874; 6,797,514; 6,867,041 and references such as Lapateva et al. (2014) Crit Rev Oncog 19(1-2):121-132; Tam et al. (2003) Cytotherapy 5(3):259-272; Garcia-Marquez et al. (2014) Cytotherapy 16(11):1537-1544. Stimulation with the FLT3, PD-1, or PD-L1 antigen ex vivo can activate and expand the selected CAR expressing cell subpopulation. Alternatively, the cells may be activated in vivo by interaction with FLT3, PD-1, or PD-L1 antigen.

In the case of certain immune cells, additional cell populations, soluble ligands and/or cytokines, or stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK-cells.

Methods of activating relevant cells are well known in the art and can be readily adapted to the present application; an exemplary method is described in the examples below. Isolation methods for use in relation to this disclosure include, but are not limited to Life Technologies Dynabeads® system activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

Further described herein is an isolated complex comprising, or alternatively consisting essentially of, or yet further consisting of the isolated cell of this disclosure bound to a cell expressing FLT3 and/or PD-1 and/or PD-L1 and/or a fragment thereof. In another aspect, the isolated complex comprises, or alternatively consists essentially of, or yet further consists of the isolated cell of this disclosure bound to FLT3 and/or PD-1 and/or PD-L1 and/or a fragment thereof.

### Compositions and Carriers

Additional aspects of the disclosure relate to compositions comprising, or alternatively consisting essentially of, or yet further consisting of, a carrier and one or more of the products—e.g., a FLT3 CAR, an isolated cell comprising a FLT3 CAR and a PD-1 and/or PD-L1 antigen binding region, a population of the cells, an isolated nucleic acid, a vector, an isolated cell containing the polynucleotide encoding the FLT3 CAR and the antibody or antigen binding fragment thereof, optionally bispecific antibody. The carrier may be a pharmaceutically acceptable carrier. In one aspect, provided herein is a composition comprising, or alternatively consisting essentially of, or yet further consisting of the isolated nucleic acid or vector, the antibody, the antigen binding fragment, the polypeptide, the isolated cell and/or the population of cells disclosed herein, and, optionally, a pharmaceutically acceptable carrier.

In further aspects, the composition may additionally comprise an immunoregulatory molecule and/or an isolated nucleic acid comprising a polynucleotide encoding an antibody or antigen binding fragment thereof, which optionally recognizes and binds PD-1 and/or PD-L1. In certain embodiments, the antibody or antigen binding fragment thereof comprises, or alternatively consists essentially thereof, or further consists of the relevant CDR regions of an antibody to PD-1 and/or an antibody to PD-L1, or an equivalent of each thereof and/or said antibody or antigen binding fragment thereof. In certain embodiments, the antibody or antigen binding fragment thereof comprises, or alternatively consists essentially thereof, or further consists of the relevant CDR regions of an antibody to PD-1 and/or an antibody to PD-L1, or an equivalent of each thereof. In some embodiments, the antibody or antigen binding fragment thereof comprises, or alternatively consists essentially thereof, or further consists of the heavy chain and/or light chain variable region of an antibody to PD-1 and/or PD-L1, and/or an equivalent of each thereof. In some embodiments, the antibody or antigen binding fragment thereof comprises a single chain variable fragment (scFv) comprising an antigen binding domain of a PD-1 antibody and/or a single chain variable fragment (scFv) comprising an antigen binding domain of a PD-L1 antibody, and/or an equivalent each thereof. In some embodiments, the scFv comprises an amino acid sequence encoded by the polynucleotide sequence:

Anti-PD-1 scFv:
(SEQ ID NO: 14)
CAGGTCCAATTGGTACAGAGCGGCGTCGAAGTAAAGAAGCCTGGAGCCAG
CGTTAAAGTTTCTTGCAAGGCTTCAGGATATACTTTCACTAACTACTATA
TGTACTGGGTACGGCAGGCTCCAGGGCAAGGGTTGGAGTGGATGGGAGGG
ATCAATCCTTCTAACGGCGGCACTAACTTTAACGAAAAATTTAAAAATAG
GGTGACCCTCACAACTGACTCAAGTACGACTACAGCATACATGGAACTCA
AATCTCTCCAATTCGATGACACGGCTGTCTATTATTGCGCGAGAAGAGAC
TATCGCTTCGATATGGGGTTTGATTATTGGGGGCAAGGTACTACGGTTAC
CGTCAGCTCCGGGGGTGGCGGCTCCGGCGGCGGTGGGTCAGGTGGAGGAG
GGTCTGACATTCAGATGACGCAATCCCCAAGCTCTCTGTCCGCGTCAGTG
GGCGACCGAGTTACAATCACATGCCGCGCTTCTCAAGATGTGTCAACCGC
TGTCGCCTGGTACCAACAGAAGCCTGGGAAGGCCCCTAAGCTTCTCATCT
ACTCAGCTTCTTTTCTGTACTCAGGGGTACCGTCTAGATTCTCAGGATCC
GGTAGTGGGACGGACTTCACATTGACCATAAGTTCCTTGCAGCCTGAGGA
TTTCGCTACATATTATTGCCAACAGTACCTTTACCATCCTGCCACTTTTG
GCCAGGGTACTAAGGTCGAGATCAAACGG, or an equivalent
thereof.

In some embodiments, the antibody or antigen binding fragment thereof comprises a single chain variable fragment (scFv) derived from an antibody to PD-L1 comprises an amino acid sequence encoded by the polynucleotide sequence:

Anti-PD-L1 scFv:
(SEQ ID NO: 16)
GAAGTTCAGTTGGTCGAGTCAGGAGGAGGCCTGGTGCAACCCGGGGGCTC
ACTCCGGTTGTCCTGTGCTGCTTCAGGATTTACGTTTTCTGACTCATGGA
TACATTGGGTGCGCCAAGCCCCGGGCAAGGGGCTGGAATGGGTGGCCTGG
ATCTCTCCGTATGGGGGTTCCACCTACTATGCTGATTCAGTAAAAGGACG
GTTCACTATAAGCGCGGATACAAGTAAGAATACTGCCTATCTTCAAATGA
ATTCTCTTCGCGCCGAGGATACAGCGGTATATTATTGCGCTAGACGACAT
TGGCCAGGGGGCTTTGACTATTGGGGGCAGGGTACTCTTGTGACCGTTAG
TGCGGGAGGTGGTGGCAGCGGTGGAGGCGGCTCCGGGGGTGGTGGTTCAG
AAATTGTCCTGACTCAATCCCCTGCCACATTGAGTTTGAGCCCAGGAGAG
AGAGCAACTCTGTCATGCCGGGCGTCAAAAGGTGTCAGTACGTCAGGCTA
CTCCTATCTTCATTGGTATCAGCAGAAACCGGGAGAAGCGCCGCGCCTTC
TCATATACCTGGCTAGTTACCTTGAGAGTGGCGTCCCGGCCCGGTTTAGT
GGGAGTGGGTCTGGGACTGATTTTACGCTGACAATCAGCAGTCTTGAGCC
AGAGGACTTCGCGGTTTACTATTGCCAACATTCACGCGATTTGCCCCTCA
CCTTCGGCGGTGGAACGAAGGTTGAAATAAAA, or an equivalent
thereof.

In some embodiments, the antibody or antigen binding fragment thereof is a bispecific antibody. In certain embodiments, the bispecific antibody comprises, or alternatively consists essentially thereof, or further consists of the relevant CDR regions of an antibody to PD-1 and/or an antibody to PD-L1, or an equivalent of each thereof. In certain embodiments, the bispecific antibody comprises, or alternatively consists essentially thereof, or further consists of the relevant CDR regions of an antibody to PD-1 and/or an antibody to PD-L1, or an equivalent of each thereof. In some embodiments, the bispecific antibody comprises, or alternatively consists essentially thereof, or further consists of the heavy chain and/or light chain variable region of an antibody to PD-1 and/or PD-L1, and/or an equivalent of each thereof. In some embodiments, the bispecific antibody comprises a single chain variable fragment (scFv) comprising an antigen binding domain of a PD-1 antibody and/or a single chain variable fragment (scFv) comprising an antigen binding domain of a PD-L1 antibody, and/or an equivalent each thereof.

In some embodiments, the composition comprises an FLT3 inhibitor. Not to be bound by theory, it is believed that such an inhibitor may increase FLT3 surface expression on the target cells. Non-limiting examples of FLT3 inhibitors include gilteritinib (Astellas), quizaritinib (Ambit Biosciences), midostaurin (Novartis), sorafenib (Bayer and Onxy Pharmaceuticals), sunitinib (Pfizer), lestarutinib (Cephalon), FF-10101 (Fuijfilm), and dovitinib (Novartis or Oncology Venture).

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of cells are administered to the host using methods known in the art and described, for example, in PCT International Application No. PCT/US2011/064191. This administration of the cells or compositions of the disclosure can be done to generate an animal model of the desired disease, disorder, or condition for experimental and screening assays.

### Methods of Use

Therapeutic Application. Method aspects of the present disclosure relate to methods for inhibiting tumor/cancer in a subject in need thereof and/or for treating a cancer patient or a subject in need thereof. Provided herein are methods of inhibiting the growth of a cancer or tumor expressing FLT3, optionally acute myeloid leukemia (AML) in a subject, comprising, or alternatively consisting essentially of, or yet further consisting of contacting the cancer or tumor with the isolated cell of or the composition of this disclosure. In one aspect, the method of inhibiting the growth of a cancer or tumor expressing FLT3 in a subject, optionally acute myeloid leukemia (AML), comprises, or alternatively consists essentially of, or yet further consists of measuring expression of PD-1 and/or PD-L1 in the subject and administering the isolated cell, the antibody, the antigen binding fragment and/or the composition of this disclosure to a subject expressing PD-1 and/or PD-L1. Further disclosed herein are methods of inhibiting the growth of a cancer or tumor in a subject, optionally acute myeloid leukemia (AML), comprising, or alternatively consisting essentially of, or yet further consisting of measuring expression of PD-1 and/or PD-L1 in the subject and administering the antibody, the antigen binding fragment and/or the composition to a subject expressing PD-1 and/or PD-L1. The contacting can be in vitro or in vivo. In some embodiments, when the contacting is in vivo and the isolated cells are autologous to a subject being treated. In other embodiments, when the contacting is in vivo, the isolated cells are allogeneic to a subject being treated. In some embodiments, the cancer is a cancer affecting blood and/or bone marrow; in some embodiments, the cancer is acute myeloid leukemia. In some embodiments, the tumor/cancer cell expresses or overexpresses FLT3 and optionally, PD-1. In certain embodiments, these methods comprise, or alternatively consist essentially of, or yet further consist of, administering to the subject or patient an effective amount of the isolated cell or compositions disclosed herein. In further embodiments, this isolated cell comprises or expresses a CAR and/or an antibody or antigen binding fragment thereof, which optionally recognizes and binds PD-1 and/or PD-L1. In certain embodiments, the antibody or antigen binding fragment thereof comprises, or alternatively consists essentially thereof, or further consists of the relevant CDR regions of an antibody to PD-1 and/or an antibody to PD-L1, or an equivalent of each thereof. In some embodiments, the antibody or antigen binding fragment thereof comprises, or alternatively consists essentially thereof, or further consists of the heavy chain and/or light chain variable region of an antibody to PD-1 and/or PD-L1, and/or an equivalent of each thereof. In some embodiments, the antibody or antigen binding fragment thereof comprises a single chain variable fragment (scFv) comprising an antigen binding domain of a PD-1 antibody and/or a single chain variable fragment (scFv) comprising an antigen binding domain of a PD-L1 antibody, and/or an equivalent each thereof. In some embodiments, the scFv comprises an amino acid sequence encoded by the polynucleotide sequence:

Anti-PD-1 scFv:
(SEQ ID NO: 14)
CAGGTCCAATTGGTACAGAGCGGCGTCGAAGTAAAGAAGCCTGGAGCCAG
CGTTAAAGTTTCTTGCAAGGCTTCAGGATATACTTTCACTAACTACTATA
TGTACTGGGTACGGCAGGCTCCAGGGCAAGGGTTGGAGTGGATGGGAGGG
ATCAATCCTTCTAACGGCGGCACTAACTTTAACGAAAAATTTAAAAATAG
GGTGACCCTCACAACTGACTCAAGTACGACTACAGCATACATGGAACTCA
AATCTCTCCAATTCGATGACACGGCTGTCTATTATTGCGCGAGAAGAGAC
TATCGCTTCGATATGGGGTTTGATTATTGGGGGCAAGGTACTACGGTTAC
CGTCAGCTCCGGGGGTGGCGGCTCCGGCGGCGGTGGGTCAGGTGGAGGAG
GGTCTGACATTCAGATGACGCAATCCCCAAGCTCTCTGTCCGCGTCAGTG
GGCGACCGAGTTACAATCACATGCCGCGCTTCTCAAGATGTGTCAACCGC
TGTCGCCTGGTACCAACAGAAGCCTGGGAAGGCCCCTAAGCTTCTCATCT
ACTCAGCTTCTTTTCTGTACTCAGGGGTACCGTCTAGATTCTCAGGATCC
GGTAGTGGGACGGACTTCACATTGACCATAAGTTCCTTGCAGCCTGAGGA
TTTCGCTACATATTATTGCCAACAGTACCTTTACCATCCTGCCACTTTTG
GCCAGGGTACTAAGGTCGAGATCAAACGG, or an equivalent
thereof.

In some embodiments, the antibody or antigen binding fragment thereof comprises a single chain variable fragment (scFv) derived from an antibody to PD-L1 comprises an amino acid sequence encoded by the polynucleotide sequence:

Anti-PD-L1 scFv:
(SEQ ID NO: 16)
GAAGTTCAGTTGGTCGAGTCAGGAGGAGGCCTGGTGCAACCCGGGGGCTC
ACTCCGGTTGTCCTGTGCTGCTTCAGGATTTACGTTTTCTGACTCATGGA
TACATTGGGTGCGCCAAGCCCCGGGCAAGGGGCTGGAATGGGTGGCCTGG
ATCTCTCCGTATGGGGGTTCCACCTACTATGCTGATTCAGTAAAAGGACG
GTTCACTATAAGCGCGGATACAAGTAAGAATACTGCCTATCTTCAAATGA
ATTCTCTTCGCGCCGAGGATACAGCGGTATATTATTGCGCTAGACGACAT
TGGCCAGGGGGCTTTGACTATTGGGGGCAGGGTACTCTTGTGACCGTTAG
TGCGGGAGGTGGTGGCAGCGGTGGAGGCGGCTCCGGGGGTGGTGGTTCAG
AAATTGTCCTGACTCAATCCCCTGCCACATTGAGTTTGAGCCCAGGAGAG
AGAGCAACTCTGTCATGCCGGGCGTCAAAAGGTGTCAGTACGTCAGGCTA
CTCCTATCTTCATTGGTATCAGCAGAAACCGGGAGAAGCGCCGCGCCTTC
TCATATACCTGGCTAGTTACCTTGAGAGTGGCGTCCCGGCCCGGTTTAGT
GGGAGTGGGTCTGGGACTGATTTTACGCTGACAATCAGCAGTCTTGAGCC
AGAGGACTTCGCGGTTTACTATTGCCAACATTCACGCGATTTGCCCCTCA
CCTTCGGCGGTGGAACGAAGGTTGAAATAAAA, or an equivalent
thereof.

In some embodiments, the antibody or antigen binding fragment thereof is a bispecific antibody. In certain embodiments, the bispecific antibody comprises, or alternatively consists essentially thereof, or further consists of the relevant CDR regions of an antibody to PD-1 and/or an antibody to PD-L1, or an equivalent of each thereof. In certain embodiments, the bispecific antibody comprises, or alternatively consists essentially thereof, or further consists of the relevant CDR regions of an antibody to PD-1 and/or an antibody to PD-L1, or an equivalent of each thereof. In some embodiments, the bispecific antibody comprises, or alternatively consists essentially thereof, or further consists of the heavy chain and/or light chain variable region of an antibody to PD-1 and/or PD-L1, and/or an equivalent of each thereof. In some embodiments, the bispecific antibody comprises a single chain variable fragment (scFv) comprising an antigen binding domain of a PD-1 antibody and/or a single chain variable fragment (scFv) comprising an antigen binding domain of a PD-L1 antibody, and/or an equivalent each thereof.

In still further embodiments, the isolated cell is a T-cell or an NK-cell. In some embodiments, the isolated cell is autologous or allogeneic to the subject or patient being treated. In a further aspect, the tumor/cancer expresses FLT3 and the subject has been selected for the therapy by a diagnostic, such as the one described herein. The subject is an animal, a mammal, a canine, a feline, a bovine, an equine, a murine or a human patient.

The FLT3 CAR cells as disclosed herein may be administered either alone or in combination with the antibody or antigen binding fragment thereof, optionally the bispecific antibody, disclosed herein, diluents, known anti-cancer therapeutics, and/or with other components such as cytokines or other cell populations that are immunoregulatory. They can be administered as a first line therapy, a second line therapy, a third line therapy, or further therapy. Non-limiting examples of additional therapies include cytoreductive therapy, such as surgical rescission, radiation therapy, cryotherapy, or chemotherapy, or biologics, such as hematopoietic stem cell transplantation. Accordingly, in some embodiments, the methods of inhibiting the growth of a cancer or tumor disclosed herein may further comprise, or alternatively consist essentially thereof, or further consist of administering to the subject an effective amount of a cytoreductive therapy. In one aspect, the cytoreductive therapy comprises, or alternatively consists essentially thereof, or further consists of chemotherapy, cryotherapy, hyperthermia, targeted therapy, and/or radiation therapy. In some embodiments, the FLT3 CAR cells may be administered before or after any one of these non-limiting exemplary therapies, e.g., before hematopoietic stem cell transplantation or after radiation therapy or chemotherapy. In embodiments where the FLT3 CAR cells are used before hematopoietic stem cell transplantation, the FLT3 CAR cells may be used to achieve remission prior to the delivery of hematopoietic stem cells; in general, hematopoietic stem cell transplantation is more successful after remission. Further non-limiting examples include other relevant cell types, such as unmodified immune cells, modified immune cells comprising vectors expressing one or more immunoregulatory molecules, or CAR cells specific to a different antigen than those disclosed herein. As with the CAR cells of the present disclosure, in some embodiments, these cells may be autologous or allogeneic. Appropriate treatment regimens will be determined by the treating physician or veterinarian.

The methods can be personalized by first identifying the patient to receive the therapy. In one aspect, the subject or patient is an animal, a mammal, a canine, a feline, a bovine, an equine, a murine or a human patient. In this aspect, a sample of cancer or tumor cells are isolated from the patient to determine if the cell expresses FLT3 and/or PD-1 and/or PD-L1. If the cell is determined to express one or more of these markers, the patient is selected to receive the therapy and the therapy is administered. Methods to determine expression of the markers are known in the art. Some such methods are described herein.

In some embodiments, the FLT3 CAR cells are administered with an FLT3 inhibitor. Not to be bound by theory, it is believed that such an inhibitor may increase FLT3 surface expression on the target cells. Non-limiting examples of FLT3 inhibitors include gilteritinib (Astellas), quizaritinib (Ambit Biosciences), midostaurin (Novartis), sorafenib (Bayer and Onxy Pharmaceuticals), sunitinib (Pfizer), lestarutinib (Cephalon), FF-10101 (Fuijfilm), and dovitinib (Novartis or Oncology Venture).

In certain embodiments, the patient or subject maintains or recovers normal hematopoiesis after receiving, i.e., being administered, the effective amount of the isolated cell. Normal hematopoiesis is a critical endpoint for certain cancers, such as but not limited to cancers affecting the blood or bone marrow, e.g., lymphoma or leukemia, such as but not limited to acute myeloid leukemia or acute lymphoblastic leukemia. Methods of determining “normal hematopoiesis” after treatment are known in the art and include but are not limited to a “pin prick” blood test comparing baseline blood cell counts to post-treatment blood cell counts and/or similar comparisons for circulating CD34+ cells. Further non-limiting exemplary methods include bone marrow biopsy to verify engraftment. Failure to maintain or recover normal hematopoiesis (also known as normal engraftment) is associated with recurrent need for transfusions and/or need for antibiotics and/or high morbidity and mortality, in addition to symptomatic indicators such as but not limited to anemia, paleness, orthostatic hypotension, and bleeding and/or bruising due to a lack of platelet recovery. Normal hematopoiesis and/or engraftment may be defined by a clinically acceptable threshold, such as but not limited to a sustained granulocyte count of >1.0×10⁹/L, a sustained platelet count of >50×10⁹, a sustained hemoglobin level of ˜9 or 10 g/dL, and/or the absence of a need for red blood cell transfusions. In some embodiments, normal hematopoiesis is defined by a lack of significant depletion of Lin−CD34+CD38−CD90+CD45RA− cells. In some embodiments, adequate long-term hematopoiesis or successful long-term hematopoietic engraftment can be correlated with sufficient numbers of Lin−CD34+CD38−CD90+CD45RA− cells in the hematopoietic product being infused into a subject following myeloablative preparation for stem cell transplantation.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials. In one aspect they are administered directly by direct injection or systemically such as intravenous injection or infusion.

The total dose of CAR expressing cells may vary depending on, for example, the above disclosed factors. In some embodiments, the doses may be on the order of between 1 to 10¹⁰ cells, e.g., at least 10, at least 10¹, at least 10², at least 10³, at least 10⁴, at least 10⁵, at least 10⁶, at least 10⁷, at most 10⁸, at most 10⁹, at most 10¹⁰, between 10² and 10¹⁰, between 10³ and 10⁹, between 10⁴ and 10⁸. In some embodiments, the dose may be further limited by an integer coefficient to the order of magnitude, e.g., 1, 2, 3, 4, 5, 6, 7, 8, or 9, resulting a dose range listed according to the following non-limiting example: between 5×10⁴ and 1×10⁸.

Suicide Gene. In embodiments involving a suicide gene as part of the isolated nucleic acid sequence encoding the CAR, the suicide gene may be utilized to terminate CAR expressing cells at the end of therapy. In method aspects involving CAR expressing cells comprising the suicide gene, the suicide gene may be induced through the introduction of the inducer molecule at the point at which the FLT3 specific CAR cell response is no longer needed. The induction of the suicide gene results in apoptosis of the CAR cells. It is thus contemplated that the use of CAR constructs comprising an inducible suicide gene may enhance the safety of CAR cell application by removing the CAR expressing cells through induced apoptosis. In embodiments where an inducing agent is used, such as but not limited to a small molecule, the dose of the inducing agent applied to induce suicide expression may range anywhere between 0.001 to 10.0 mg/kg body weight, or alternatively from 0.01 to 1.0 mg/kg, and ranges in between.

Diagnostic Applications. Aspects of the disclosure provide an exemplary method for determining if a patient is likely to respond to, or is not likely to respond to, FLT3 CAR therapy. In specific embodiments, this method comprises contacting a biological sample isolated from the patient with an effective amount of an anti-FLT3 and/or PD-L1 and/or PLD1 antibody and detecting the presence of any antibody bound to the cancer/tumor sample. In some embodiments, the tumor sample is any biological sample including cancer/tumor cells, e.g., a tumor biopsy, circulating cancer/tumor cells, and/or any other bodily fluid or tissue that may comprise the cells. In further embodiments, the presence of antibody or antibodies bound to the cancer/tumor sample indicates that the patient is likely to respond to the FLT3 CAR therapy and the absence of antibody bound to the tumor sample indicates that the patient is not likely to respond to the FLT3 CAR therapy. In some embodiments, the antibody may bind between 0% and 100% of the cancer/tumor sample taken from the patient may comprise cells that are FLT3 positive; in such embodiments, it is to be understood that the higher the percentage of FLT3 and/or PD-1 and/or PD-L1 positive tumor cells, the higher likelihood that FLT3 CAR therapy will be effective. In some embodiments, the cancer/tumor sample comprises leukemic blasts. In further embodiments, detection of greater or about 90% of leukemic blasts expressing FLT3 and/or detection of at least 50% of PD-1 and/or PD-L1 indicates a patient has a favorable “therapeutic window” for FLT3 CAR therapy. In some embodiments, the method involves the use diagnostic assays, markers, or gene expression profiles associated with a tumor or cancer—a non-limiting exemplary is quantifying the population of cells expressing CD45^{dim}SSC^{medium} using flow cytometry or another cell sorting method to determine if there has been a reduction of AML relative to the baseline population of these same cells. In some embodiments, the method comprises the additional step of administering an effective amount of the FLT3 CAR expressing cells and the antibody disclosed herein to a patient that is determined likely to respond to the FLT3 CAR therapy. In some embodiments, the patient has and/or is diagnosed with a FLT3 expressing cancer/tumor. In some embodiments, the cancer/tumor is a lymphoma or leukemia, such as but not limited to AML or ALL.

### Kits

As set forth herein, the present disclosure provides methods for producing and administering FLT3 CAR cells and an antibody or antigen binding fragment thereof comprising an antigen binding domain that recognizes and binds PD-1 and/or PD-L1. In one particular aspect, the present disclosure provides kits for performing these methods as well as instructions for carrying out the methods of the present disclosure such as collecting cells and/or tissues, and/or performing the screen/transduction/etc., and/or analyzing the results.

In one aspect the kit comprises, or alternatively consists essentially of, or yet further consists of, any one of the isolated nucleic acids disclosed herein and/or one or more vectors comprising said nucleic acid and/or isolated allogeneic cells, preferably T-cells or NK-cells, and/or instructions on the procuring of autologous cells from a patient. In another aspect, disclosed herein are kits comprising, or alternatively consisting essentially of, or yet further consisting of the composition as disclosed herein and optionally, instructions for use. Such a kit may also comprise, or alternatively consist essentially of, or yet further comprise media and other reagents appropriate for the transduction and/or selection and/or activation and/or expansion of FLT3 CAR expressing cells, such as those disclosed herein.

In one aspect the kit comprises, or alternatively consists essentially of, or yet further consists of, an isolated CAR expressing cell or population thereof. In some embodiments, the cells of this kit may require activation and/or expansion prior to administration to a subject in need thereof. In further embodiments, the kit may further comprise, or consist essentially thereof, media and reagents, such as those covered in the disclosure above, to activate and/or expand the isolated CAR expressing cell. In some embodiments, the cell is to be used for FLT3 CAR therapy. In further embodiments, the kit comprises instructions on the administration of the isolated cell to a patient in need of FLT3 CAR therapy.

The kits of this disclosure can also comprise, e.g., a buffering agent, a preservative or a protein-stabilizing agent. The kits can further comprise components necessary for detecting the detectable label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

The following examples are illustrative of procedures which can be used in various instances in carrying the disclosure into effect.

### Example 1—FLT3 Car and Secreted PD-1/PD-L1

FLT3 CAR T-cells not only induce a time-dependent and dose-dependent cytotoxicity against FLT3(+) AML cell line, but also kill up to 40% FLT3(+) primary AML patient blasts in as little as 4 hours. More importantly, Applicant had found that CAR NK-cells or NK-cell in prolonged culture expressed substantial amounts of checkpoint protein PD-1, which is an inhibitory signal on cancer patient NK-cells, whereas the AML blasts expressed PD-L1 on cell surface. From this previous bispecific platforms and other groups, the major concern of bispecific antibody (“biAb”) is the short half-life, limiting the bioavailability and efficacy. Therefore, Applicant sought to overcome this technical limitation and provide a synergistic cytolytic activity against AML through increased engagement, increased activation and antagonizing checkpoints inhibition.

### Efficacy of CAR NK-Cells

The efficacy of the CAR NK-cells constitutively secreting anti-PD-1-PD-L1 biAb can be assessed in vitro. FLT3 CAR-NK clones with continuous and high secretion of PD-1-PD-L1 bispecific antibody have been generated and an increase in cytotoxicity against AML cell lines and patient blasts can be tested in vitro.

To do so, an anti-PD-1-PD-L1 biAb was generated and incorporated into the FLT3 CAR lentiviral vector. The biAb contains scFvs from anti-PD-1 and anti-PD-L1 antibodies with a HMA linker. The anti-PD-1 scFv-HMA-anti-PD-L1 scFv fragment expression is driven under CMV promoter and secretion signal peptide isolated from human immunoglobulin molecules.

In order to test the efficacy of the CAR NK-cells constitutively secreting anti-PD-1-PD-L1 biAb in vitro, FLT3 CAR carrying novel anti-PD-1-PD-L1 bispecific antibodies are transduced into cells. Since a single gene transfer via lentiviral system is better than two rounds of transduction in term of efficacy and safety, a clinical grade bicistronic expression vector expressing both FLT3 and the biAb is preferred. CAR T-cells transduced with such bicistronic FLT3 vectors induce potent and specific cytolytic activities against FLT3(+) AML cells. Not to be bound by theory, FLT3 CAR are believed to redirect NK-cells against FLT3(+) targets while anti-PD-1-PD-L1 biAb engage both effector and target and act as a checkpoint inhibitor.

The bicistronic expression vector and induction of cell surface expression of FLT3 CAR and continuous secretion of anti-PD-1-PD-L1 bispecific antibody can also be assessed The half-life and bioavailability of biAb is assessed due to its small size. Not to be bound by theory, insertion of the secretory biAb gene into FLT3 CAR vector is believed improve the half-life and bioavailability of the bispecific antibody.

FLT3 CAR expression can be determined by staining with anti-Fab antibody and flow cytometry. The protein expression of the biAb is assessed by blocking the secretory pathway with brefeldin A and the cell is permeabilized and stained with anti-6×Histidine (6×His (SEQ ID NO: 17)) antibody. An ELISA screen for 6×His (SEQ ID NO: 17) can be used to screen out the high expresser candidates for anti-PD-1-PD-L1 biAb. FLT3 CAR are detected on the cell surface. When transduced cells are stained intracellularly for anti-6×His antibody (“6×His” disclosed as SEQ ID NO: 17), the biAb is detected by flow cytometry and quantified with ELISA. At least one higher expresser of both FLT3 CAR and anti-PD-1-PD-L1 biAb is preferred and selected. Applicant notes that, in some cases, artificial expression of the two proteins might stress the cells and initiate protein misfolding response and massive cell death might occur. This can be overcome by the use of clinically safe promoters with different driving strength to optimize expression.

Applicant also can determine whether FLT3 CAR-PD-1-PD-L1 biAb treatment better than FLT3 CAR alone in vitro. FLT3 CAR induces up to 40% cytotoxicity against patient AML blasts. The level of cytotoxicity can further be augmented if inhibitory checkpoints are blocked by the biAb.

A side-by-side comparison of cytotoxicity against the AML cell line MOLM-13, K562 and U937, which have the high, intermediate and low expression of PD-L1, respectively to compare the cytotoxicity with or without CAR or anti-PD-1-PD-L1 biAb can also be assessed. The IFN-γ production in co-cultures with or without CAR or bispecific antibody can be measured and compared. To prove the engagement, the pre-labelled effector cells, which overexpress secretory biAb insert (anti-PD-1-PD-L1 CAR NK-cells), and pre-labelled AML cells are co-cultured for 30 min. The cells are then harvested and stained with a fluorophore labelled with phalloidin. The stained cells are analyzed with an Imagestream X Marker II imaging flow cytometer. The frequency of immune-synapse is quantified and compared with control cells after stringent gating strategy. To determine the effect of blocking PD-1 on NK-cells and CAR signaling pathways, phosphorylation of SHP2 (downstream of PD-1 signaling), AKT (downstream of CD28 signaling), and zap70, downstream of TCR signaling are measured. The interaction of endogenous PD-1 expression on CAR NK-cells and PD-L1 expression on AML cells is antagonized and an increase in specific lysis is observed in co-cultures in the combined treatment group. The frequency of immune-synapse in co-culture with anti-PD-1-PD-L1 CAR NK-cells is higher than the negative control including empty vector control and no target control. The lower expression of SHP2 is detected in the CAR NK with biAb, whereas the AKT and zap70 will be phosphorylated, after the treatment of anti-PD-1-PD-L1 biAb. While NK-cells or T-cells express both PD-1 and PD-L1 and, thus, anti-PD-1-PD-L1 biAb may engage the PD-1 or PD-L1 on the effector cells, the CAR on the effector cells retarget only to the FLT3(+) cell which are limited to malignant AML and myeloid cells. This provides specificity and safety.

### CAR-NK Bispecific Cells Prolong Survival

CAR-NK clones (NK92) with secreted PD-1-PD-L1 (biAb-FLT3 CAR NK) were tested to determine if the cells prolong survival in AML patient derived xenograft and leukemic stem cell mouse models. Applicant has found that AML mice treated with CAR-NK with secreted PD-1-PD-L1 biAb survive longer than those with leukemia alone or treated with CAR alone.

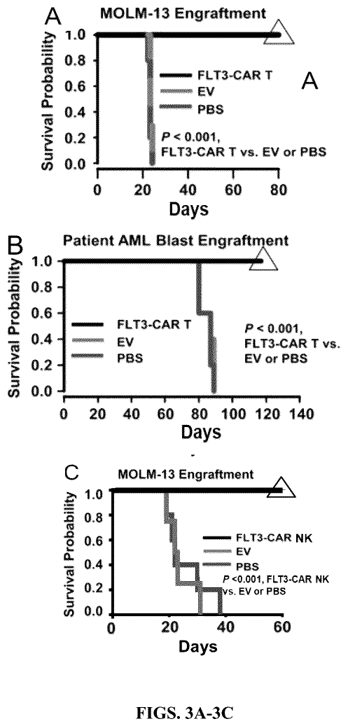

Applicant also assessed whether FLT3 CAR NK-cell induced significant cytolytic activity among AML cell lines and patient blasts. FLT-3 CARs are constructed and expressed it on human primary CD3(+) T and NK-cells. The CAR NK-cells specifically killed FLT3(+) AML cell lines MOLM-3, and EOL-1, but not the negative control cell line U937 (FIG. 2A). Similarly, the CAR T-cells induced cytolytic activity against FLT3(+) blasts from AML patients, but not FLT3(−) AML blasts (FIG. 2B). There is no significant killing against human normal cells including CD34(+) haemopoietic stem cells, dendritic cells, NK and B-cells, although they expressed FLT-3 (FIG. 2C). This suggested FLT3 CAR T- or NK-cells may be safe.

FLT3 CAR T-cells prolong survival of mouse bearing MOLM-13 AML cells and primary AML blasts from patients. NOD-SCID^{IL-2 γc-/-} mice injected with AML cell lines or primary AML blasts were used as AML patient blasts models. All the mice bearing MOLM-13 cells and FLT3 CAR T-cells survived over 80 days post MOLM-13 AML cell implantation (FIG. 3A), compared to control mice that received empty vector-transduced T-cells that died approximately 20 days post MOLM-13 AML cell implantation. Similarly, mice with human AML blast alone died at around 90 days. In contrast, those received FLT3 CAR T-cells survived for more than 120 days without symptoms of AML (FIG. 3B). Applicant's data shows that FLT3 CAR is expressed on human NK-cells. Similar results have been determined for FLT3 CAR NK-cells (FIG. 3C).

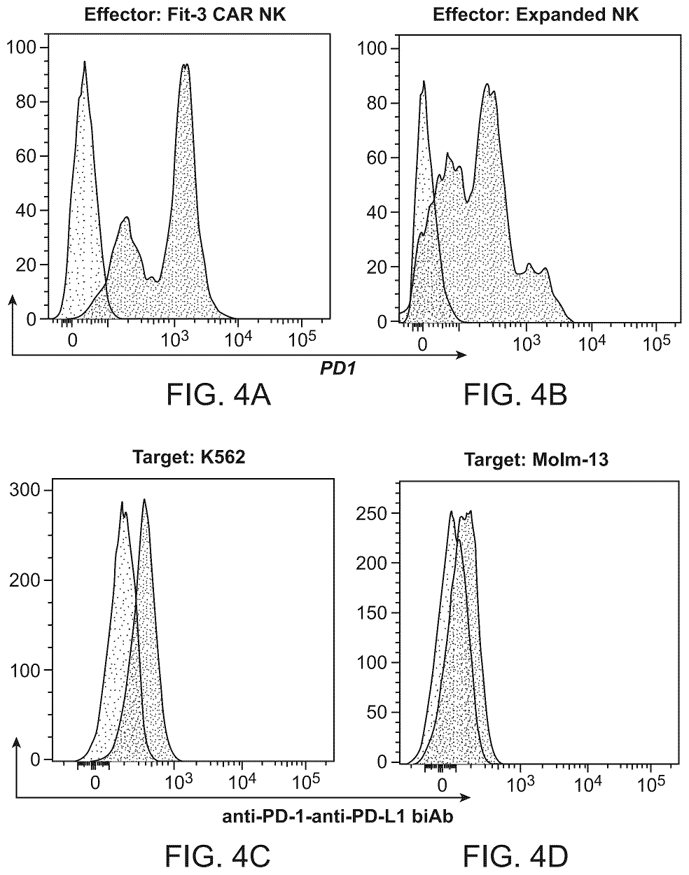

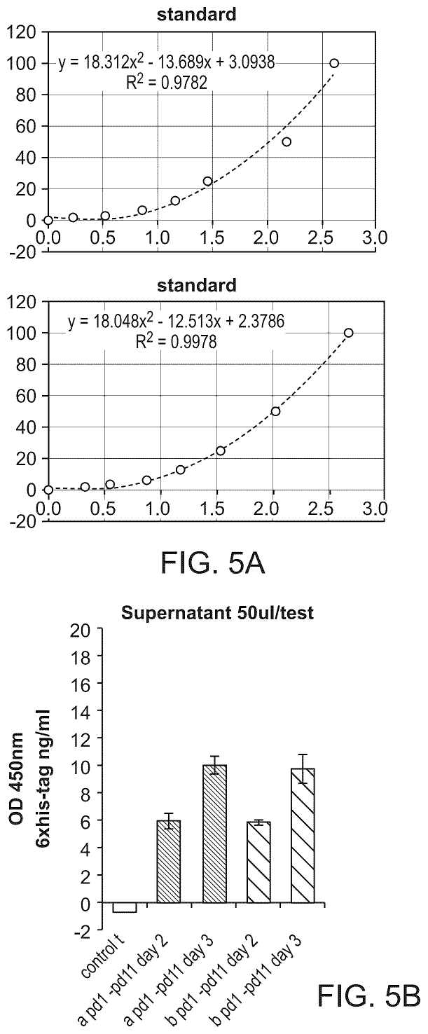

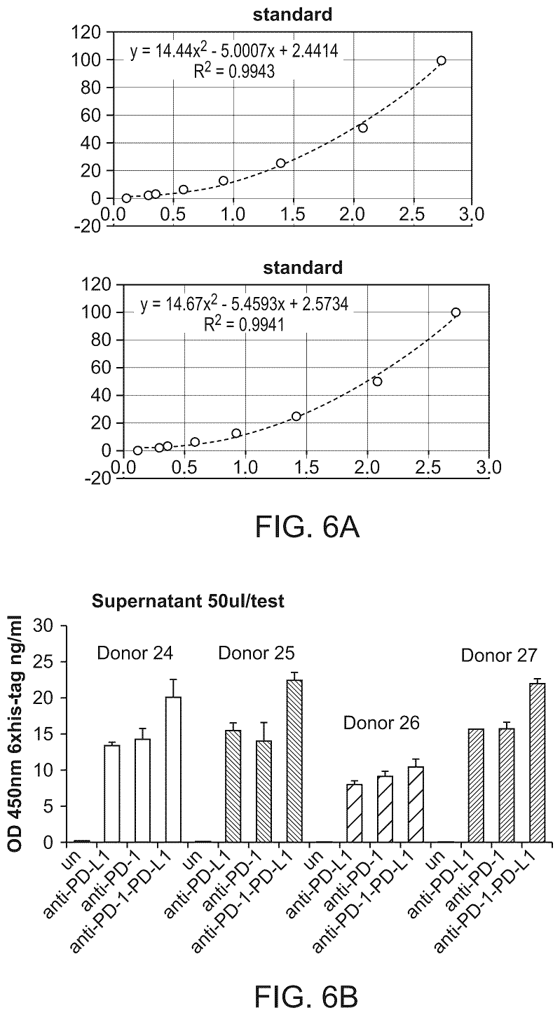

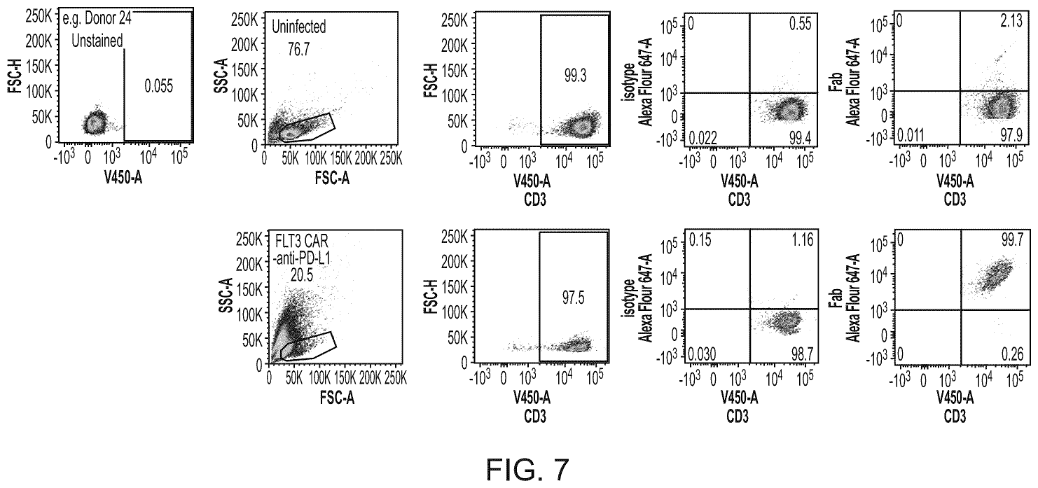

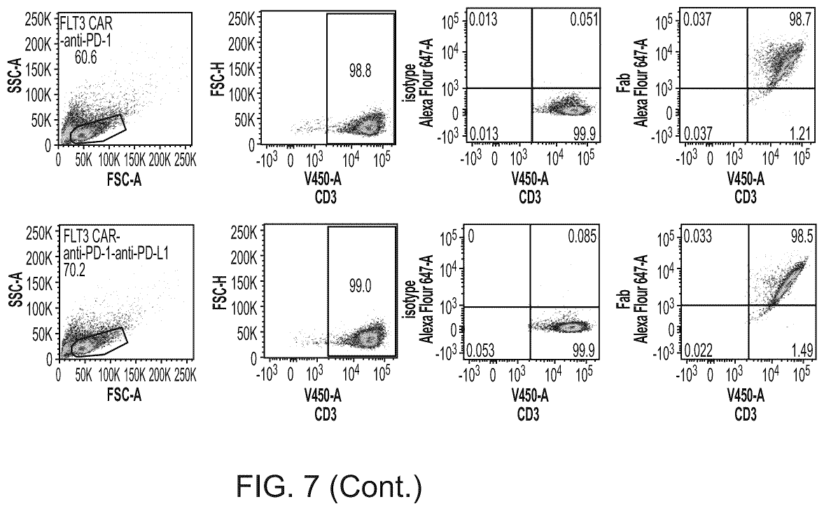

FLT3 CAR T-cells express PD-1 during expansion phase, and AML blasts express PD-L1. Applicant found that FLT3 CAR NK-cells express a high level of PD-1 in culture. The percentage of PD-1(+) CAR NK-cells was over 90% after 7 days in culture (FIG. 4A). Primary NK-cells without CAR transduction also expressed PD-1 in the presence of gamma chain cytokines (FIG. 4B). In addition, AML cell lines express PD-L1 at differing levels. The high expressers include K562; and the low expressers include Molm-13 (FIG. 4C-D). These results suggest that PD-1-PD-L1 axis plays a potential role in the cytotoxicity of CAR-NK against AML cells. Applicant also found that 10 ng/mL biAb could be detected in the culture supernatants of the T-cells transduced with biAb vector 3 days post-transduction (FIG. 4E).

These results demonstrated the FLT3 CAR T or NK-cells could protect most of the AML mice from death. Not to be bound by theory, it is believed that by antagonizing PD-1 and PD-L1 with secreted biAb, the CAR NK-cells protect mice from AML LSC (Leukemia stem cells in AML).

Applicant also tested whether FLT3 CAR-PD-1-PD-L1 biAb treatment better than FLT3 CAR alone in AML mouse. The data shows that although the FLT3 CAR T-cells can prolong survival in AML mice model, the CAR T-cells express a high level of PD-1. Applicant also detected increased PD-1 expression in CAR NK-cells. Therefore, Applicant believes that antagonizing PD-1-PD-L1 with biAb enhances the functions of CAR and eradicate AML cells in AML mouse model created using either cell line or patient derived xenograft.

Nod scid gamma (NSG) mice are inoculated with luciferase-transduced MOLM-13, a FLT3(+) AML cell line. A week after the inoculation, FLT3 CAR-NK with or without anti-PD-1-PD-L1 biAb are injected intravenously. The kinetics of injected AML cells are followed weekly by IVIS Lumina II by measuring the bioluminescence signals from AML cells. At particular time-point (guided from the in vivo imaging), Applicant harvested the blood, bone marrow and spleen, and determine the absolute number of AML and NK-cells. PD-1 expression on CAR NK-cells is determined by flow cytometry in the peripheral blood sampled every week. The experiment can be repeated by injecting primary AML patient blast samples stratified by the percentage of FLT3 expression. Expression of PD-L1 on the AML cells injected can also be determined. The concentration of anti-PD-1-PD-L1 biAb is measured regularly by ELISA CAR NK-cells with anti-PD-1-PD-L1 biAb persist or proliferate better than those without in vivo. The PD-1 or PD-L1 expression on CAR NK- and AML cells, respectively, is not detected in CAR-NK with the biAb. In addition, the bioluminescence from AML cells is lowest in biAb-FLT3 CAR NK group. A significant longer disease-free survival is shown in mice receiving biAb-FLT3 CAR NK treatment. The anti-PD-1-PD-L1 biAb are tested regularly in the plasma samples. In some instances, the effect of the biAb may depend on the effect of other immune cells in PBMCs. In such cases, Applicant can co-inject autologous PBMCs and biAb-FLT3 CAR NK-cells into the AML mouse model and compare those without injected PBMCs.

Applicant also determined whether the PD-1-PD-L1 biAb-FLT3 CAR NK-cells kill the leukemia stem cells (LSC) and protect mice from relapse. Leukemic stem cells were first described in AML as a small subset of AML blasts usually CD34(+)CD38(−)CD123(+) that could regenerate into a mass population of AML blasts. This is clinically relevant as standard treatment spares this compartment in AML, leading to relapse and resistance against the treatment in the AML patient. It is well known that enriched leukemic stem cells population carries internal tandem duplication of the FLT3 gene.

Cytotoxicity assays are performed in vitro using samples enriched for LSC. Samples from AML patients are enriched by sorting for CD34(+)CD38(−)CD123(+) cells. FLT3 CAR-NK or biAb-FLT3 CAR NK-cells are enriched by sorting for CD34(+)CD38(+) cells. Applicant also measures the IFN-γ production from the two groups of cells and compare with cells transduced with target alone/effector alone. In vivo, the NOD-SCIDIL2γc−/− mice (both female and male) are irradiated at 100 Rad the day before the leukemic cell injection. One day later, LSC as defined as CD34(+)CD38(−)CD123(+) and negative control CD34(+)CD38(+) are freshly sorted by flow cytometric cell sorter (BD Aria III) and 1×10⁶ sorted LSC or control cells will be injected via tail vein. Meanwhile, primary NK-cells are transduced with the constructs for FLT3 CAR alone, biAb NK alone, or biAb-FLT3 CAR. The mice are then randomly assigned into 5 different groups: 1. AML alone, 2. AML+NK-cell-empty vector, 3. AML+FLT3 CAR NK, 4. AML+biAb-NK, and 5. AML+biAb-FLT3 CAR-NK. 1×10⁶ transduced NK-cells are injected intravenously one day after LSC infusion. After 12-14 weeks, the engraftments are checked by sampling peripheral blood. The survival of the mice is monitored. At the end-point, the frequency of LSC is analyzed by flow cytometry. The biAb-FLT3 CAR NK-cells lyse the LSC at least as efficient as the cells in the non-LSC compartment. The treatment with both CAR and biAb prevents relapse of the AML. A longer disease-free survival is observed. In some cases, LSC may all be killed by the CAR NK-cells infused a day after infusion, rendering it impossible to determine the effect of LSC re-population. In such cases, Applicant can repeat the experiment but infuse NK-cells 12-14 weeks after LSC engraftment.

### Safety

The safety of the biAb-FLT3 CAR NK-cell is tested in vivo using humanized AML patient-derived mouse model. No severe cytokine storm was seen in mice treated with FLT3 CAR-NK and anti-PD-1-PD-L1 biAb.

Applicant observed that FLT3 CAR T-cells do not kill CD34(+) stem cells. Not to be bound by theory, Applicants believe biAb-FLT3 CAR NK preserve normal stem cells and provide specific cytotoxicity against FLT3(+) AML cell. However, since FLT3 is expressed on both myeloid and stem cells, FLT3 CAR NK-cells can be tested to determine whether normal hematopoietic stem cells are depleted and risk cytopenia.

Applicant also test whether FLT3 CAR-NK with secretory biAb will kill normal FLT3(+) cells. Not to be bound by theory, it is believed that antagonizing PD-1/PD-L1 provides a synergistic effect on CAR activation. This may increase the risk of cytotoxicity against all normal FLT3(+) cells, even if they may only weakly express FLT3. Accordingly, cytotoxicity assays are performed using biAb-FLT3 CAR NK as effectors and normal CD34(+) stem cells isolated from umbilical cord blood as target at different effector-to-target ratio.

To validate in vitro data, NSG-SGM3 (NSGS) mice that express human IL3, GM-CSF, and SCF are used. Both human CD34(+) stem cells and FLT3 CAR NK-cells or biAb-FLT3 CAR NK are injected intravenously with 5×10⁴ cells per mouse. One month or three months later, all the mice are sacrificed and the engraftment of human CD45(+) cells in bone marrow is determined. The anti-PD-1-PD-L1 biAb does not affect the FLT3 CAR NK-cells on normal hematopoietic stem cells. Furthermore, the percentage of CD45(+) cells in mice administered with FLT3 CAR-NK with secretory anti-PD-1-PD-L1 biAb is same or similar as those that receive only FLT3 CAR.

### Statistical Considerations

For all power calculations, tests are two-sided. Applicant uses log transformations for most continuous measures, and arc-sin square root transformations for percentages, both of which have excellent variance stabilizing properties for pooling variance. Even though the Bonferroni method was used to control for type-I error in the power calculations, Holm's or a more powerful method will be used in the data analyses when pairwise comparisons are performed. Kaplan-Meier curves are plotted to display the survival results. Linear mixed effect models are used to detect the trend of changes on IFN-γ production from the blood samples repeatedly taken during mouse survival study. Two sample t-tests will be used for the analyses. To study differences in the number of CAR NK-cells between groups, eight mice are included in each group, which will achieve 80% power to detect at least a 2-fold effect (α=0.05/3 to adjust for the two treatment groups and one AML alone group, CV=50%). Two sample t-test will be used for the analysis.

### DISCUSSION

Applicant's results establish how anti-PD-1-PD-L1 biAb affect the CAR-NK functions, resulting in developing a novel cellular therapeutic strengthened by CAR and anti-PD-1-PD-L1 biAb against AML. Not to be bound by theory, this approach is believed to be complementary to the persistence of CAR-NK functionality in AML microenvironment. The combination of the advantages of both CAR and biAb technologies makes the biAb more powerful in vivo. Further studies were conducted with secreted anti-PD-1 and anti-PD-L1 alone, i.e., not as a bispecific construct.

Applicant's anti-PD-1-PD-L1 biAb combines the CAR and bispecific antibody technologies, where the constitutively secreted biAb resolve potential issues in the field such as short half-life and costly protein production and purification. The anti-PD-1-PD-L1 biAb contains neutralizing capacity to checkpoints on both effector and target cells. It is unique and first in the field. Checkpoint inhibitors have systemic toxicity. In contrast, anti-PD-1-PD-L1 biAb is expected to have low toxicity because the antagonization effects limited to only cancer cell in close proximity of the effector cells. Enhancing CAR NK-cell functions by antagonizing PD-1/PD-L1 for cellular therapy is innovative and has not been reported. This study offers increased sustainability of the therapeutic effects. Applicant's studies benefit other cellular therapies using NK-cells as well. Cellular expression of PD-1-PD-L1 biAb on CAR-NK is novel. The secretory design of the PD-1-PD-L1 biAb in the expression vector render the ubiquitous secretion of PD-1-PD-L1 bispecific antibody. This bypass the protein production and administration hassle for the bispecific antibody. This in turn greatly increases the bioavailability of this small bispecific antibody.

### Example 2—PD-1 and PD-L1 Antibodies

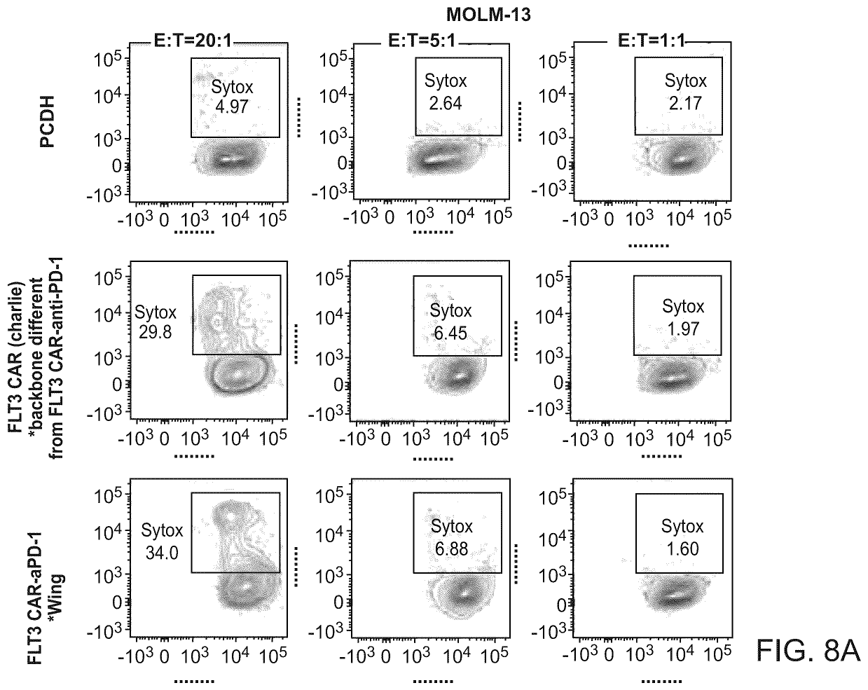

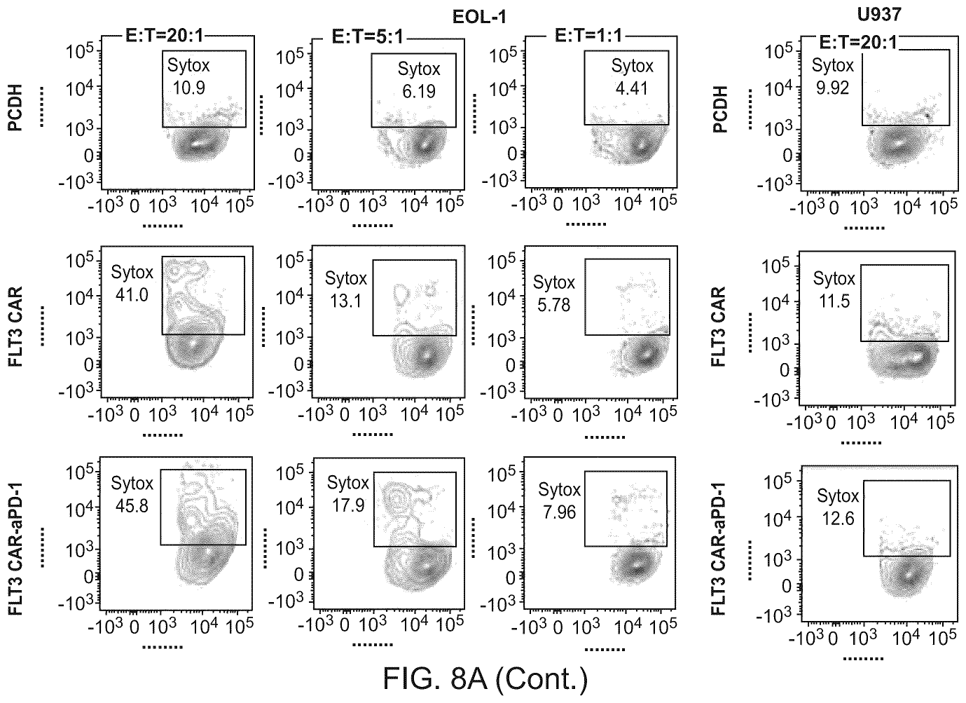

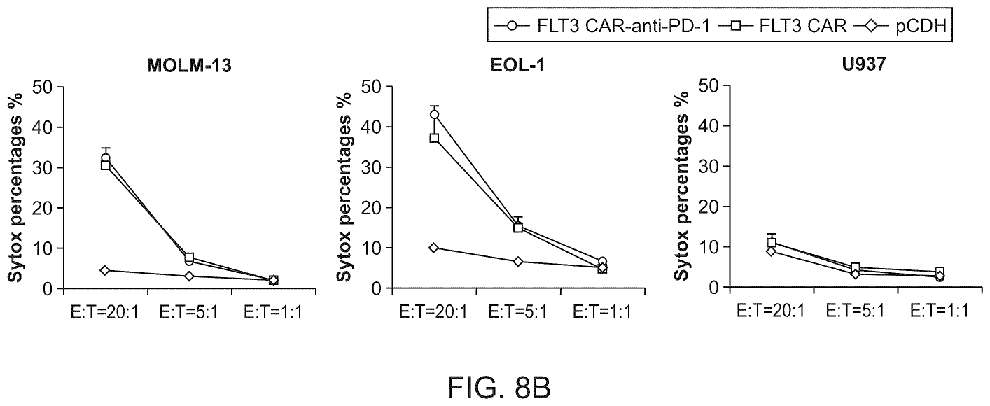

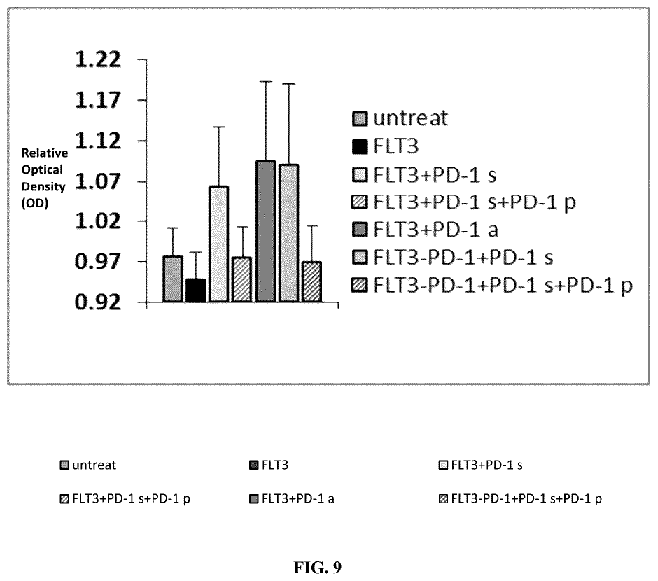

Similar success was demonstrated with PD-1 and PD-L1 antibodies (i.e. not bispecific) (FIGS. 8A-8B and FIG. 9). The above studies are replicated for PD-1 and PD-L1 antibodies (i.e. not bispecific).

### Example 3—FLT3 Inhibitor Co-Administration

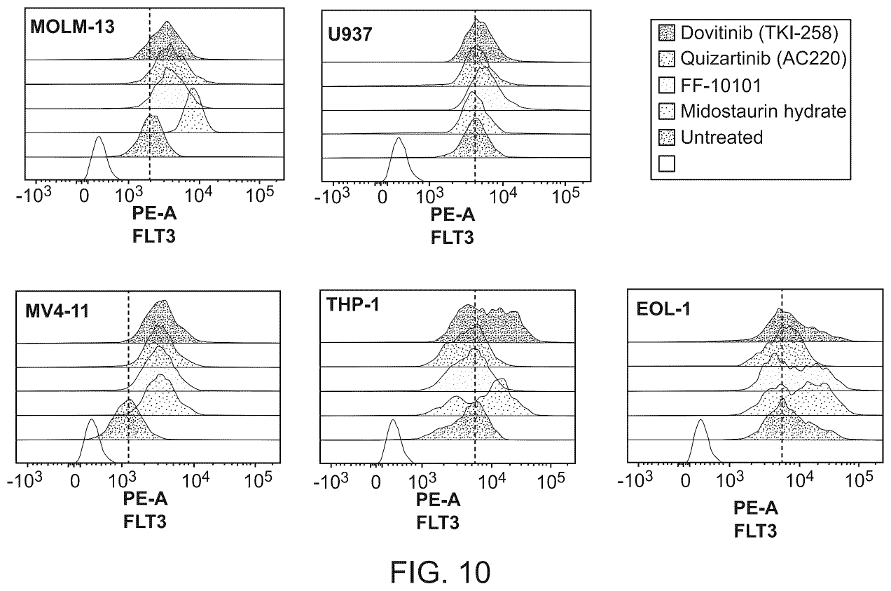

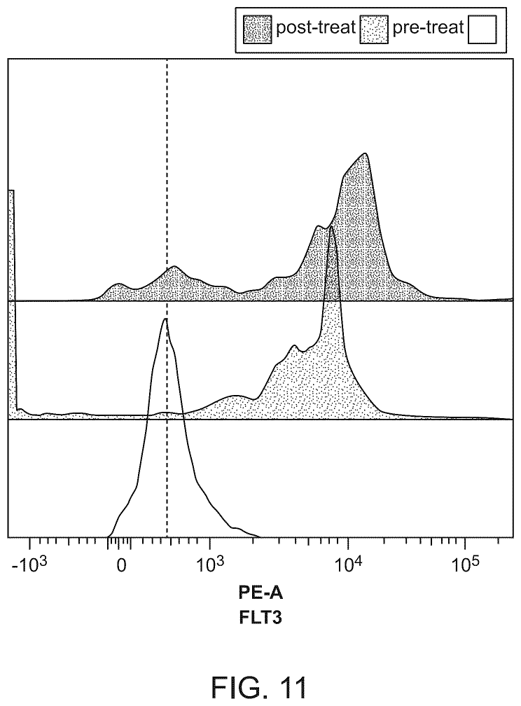

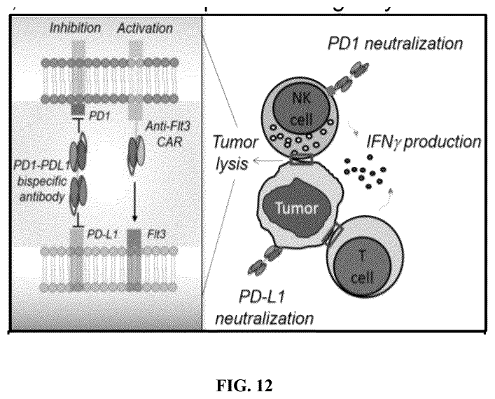

Further experiments were conducted to determine whether FLT3 expression was increased upon administration of an FLT3 inhibitor. The results are shown in FIG. 10 and FIG. 11. FIG. 10 shows the detection of surface FLT3 expression in MOLM-13, U937, THP-1, MV4-11, and EOL-1 AML cell lines treated with 10 μM of Midostaurin, FF-10101, Quizartinib (AC220), and Dovitinib (TKI-258) FFLT3 inhibitors for 48 hours. FLT3 surface expression was upregulated after treatments with FLT3 inhibitors. FIG. 11 depicts the detection of surface FLT3 expression by flow cytometry in peripheral blood samples of patients who were treated with Midostaurin for 48 hours. FLT3 surface expression was upregulated after the treatment.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. An isolated nucleic acid or a vector comprising:
a. a polynucleotide encoding a chimeric antigen receptor (CAR) comprising: (a) an antigen binding domain of an FLT3 antibody; (b) a hinge domain; (c) a transmembrane domain; (d) and an intracellular domain; and
b. a polynucleotide encoding an antibody or antigen binding fragment thereof comprising an antigen binding domain that recognizes and binds PD-1 or PD-L1 or both PD-1 and PD-L1.

2. The isolated nucleic acid or the vector of claim 1, further comprising (e) a polynucleotide encoding a signaling domain.

3. The isolated nucleic acid or the vector of claim 1, wherein the CAR further comprises an inducible or a constitutively active element, optionally wherein the inducible or the constitutively active element controls the expression of a polynucleotide encoding an immunoregulatory molecule or a cytokine.

4. (canceled)

5. The isolated nucleic acid or the vector of claim 3, wherein the immunoregulatory molecule or cytokine comprises one or more of B7.1, CCL19, CCL21, CD40L, CD137L, GITRL, GM-CSF, IL-12, IL-2, low-toxicity IL-2, IL-15, IL-18, IL-21, LEC, or OX40L, optionally wherein the immunoregulatory molecule or cytokine comprises one or more of the following: IL-12; GM-CSF; IL-12 and GM-CSF; IL-12 and one or more of IL-2 and low-toxicity IL-2; one or more of IL-2 and low-toxicity IL-2; IL-12 and IL-15; IL-15; IL-12 and IL-21; IL-21; IL-12 and B7.1; B7.1; IL-12 and OX40L; OX40L; IL-12 and CD40L; CD40L; IL-12 and GITRL; GITRL; IL-12 and IL-18; IL-18; one or more of IL-2 and low-toxicity IL-2 and one or more of CCL19, CCL21, and LEC; IL-15 and one or more of CCL19, CCL21, and LEC; IL-21 and one or more of CCL19, CCL21, and LEC; GM-CSF and one or more of CCL19, CCL21, and LEC; OX40L and one or more of CCL19, CCL21, and LEC; CD137L and one or more of CCL19, CCL21, and LEC; B7.1 and one or more of CCL19, CCL21, and LEC; CD40L and one or more of CCL19, CCL21, and LEC; or GITRL and one or more of CCL19, CCL21, and LEC.

6. (canceled)

7. The isolated nucleic acid or vector of claim 1, wherein the hinge domain comprises a CD8 α hinge domain.

8. The isolated nucleic acid or vector of claim 1, wherein the transmembrane domain comprises a CD8a transmembrane domain.

9. The isolated nucleic acid or vector of claim 1, wherein the costimulatory signaling region comprises a CD28 costimulatory signaling region or a 4-1BB costimulatory signaling region or both a CD28 costimulatory signaling region and a 4-1BB costimulatory signaling region.

10. The isolated nucleic acid or the vector of claim 1, wherein the CAR comprises: (a) an antigen binding domain of a FLT3 antibody; (b) a CD8 α hinge domain; (c) a CD8 α transmembrane domain; and (d) a CD28 costimulatory signaling region or a 4-1BB costimulatory signaling region or both a CD28 costimulatory signaling region and a 4-1BB costimulatory signaling region.

11. The isolated nucleic acid or the vector of claim 2, wherein the CAR comprises: (a) an antigen binding domain of a FLT3 antibody; (b) a CD8 α hinge domain; (c) a CD8 α transmembrane domain; (d) a CD28 costimulatory signaling region or a 4-1BB costimulatory signaling region or both a CD28 costimulatory signaling region and a 4-1BB costimulatory signaling region; and (e) a CD3 zeta signaling domain.

12. The isolated nucleic acid or vector of claim 1, wherein the antigen binding domain of the FLT3 antibody comprises a heavy chain variable region comprising:
a CDHR1 having the amino acid sequence
(SYWMH (SEQ ID NO: 1))
or
(NYGLH (SEQ ID NO: 2))
or an equivalent of each thereof,
a CDHR2 having the amino acid sequence
(EIDPSDSYKDYNQKFKD (SEQ ID NO: 3))
or
(VIWSGGSTDYNAAFIS (SEQ ID NO: 4))
or an equivalent of each thereof,
and
a CDHR3 having the amino acid sequence
(AITTTPFDF (SEQ ID NO: 5))
or
(GGIYYANHYYAMDY (SEQ ID NO: 6))
or an equivalent of each thereof,
and/or
a light chain variable region comprising:
a CDLR1 having the amino acid sequence
(RASQSISNNLH (SEQ ID NO: 7))
or
(KSSQSLLNSGNQKNYM (SEQ ID NO: 8))
or an equivalent of each thereof,
a CDLR2 having the amino acid sequence
(YASQSIS (SEQ ID NO: 9))
or
(GASTRES (SEQ ID NO: 10))
or an equivalent of each thereof,
and
a CDLR3 having the amino acid sequence
(QQSNTWPYT (SEQ ID NO: 11))
or
(QNDHSYPLT (SEQ ID NO: 12))
or an equivalent of each thereof.

13. The isolated nucleic acid or vector of claim 1, wherein the antigen binding domain that recognizes and binds PD-1 or PD-L1 or both PD-1 and PD-L1 comprises one or more of the following: a PD-1 antagonist, a PD-L1 antagonist, CDR regions of an antibody to PD-1 or PD-L1, a heavy chain and a light chain variable region of an antibody to PD-1 or PD-L1, a single chain variable fragment (scFv) comprising an antigen binding domain of a PD-1 antibody, a scFv comprising an antigen binding domain of a PD-L1 antibody, or an equivalent of each thereof.

14.-16. (canceled)

17. The isolated nucleic acid or vector of claim 1, wherein the antibody that recognizes and binds PD-1 or PD-L1 or both PD-1 and PD-L1 is a bispecific antibody,
optionally wherein the bispecific antibody comprises a PD-1 antagonist, a PD-L1 antagonist and an optional linker, or
wherein the bispecific antibody comprises CDR regions of an antibody to PD1, CDR regions of an antibody to PD-L1 and an optional linker, or
wherein the bispecific antibody comprises a heavy chain and light chain variable region of an antibody to PD1, a heavy chain and light chain variable region of an antibody to PD-L1 and an optional linker, or
wherein the bispecific antibody comprises an scFv comprising an antigen binding domain of an antibody to PD1, an scFv comprising an antigen binding domain of an antibody to PD-L1 and an optional linker.

18.-21. (canceled)

22. The isolated nucleic acid or vector of claim 1, wherein the vector is a plasmid or wherein the vector is a viral vector selected from a retroviral vector, a lentiviral vector, an adenoviral vector, or an adeno-associated viral vector, optionally wherein the vector is bicistronic or wherein the isolated nucleic acid or vector further comprises a promoter or enhancer operatively linked to the polynucleotide encoding the antibody or the antigen binding fragment that recognizes and binds PD-1 or PD-L1 or both PD-1 and PD-L1.

23.-25. (canceled)

26. An isolated cell comprising the isolated nucleic acid or vector of claim 1, wherein the cell is a prokaryotic cell or a eukaryotic cell selected from an animal cell, a mammalian cell, a bovine cell, a feline cell, a canine cell, a murine cell, an equine cell, a human cell, an immune cell, a T-cell, a B-cell, a NK-cell, a dendritic cell, a myeloid cell, a monocyte, or a macrophage, optionally wherein the eukaryotic cell has been activated.

27.-32. (canceled)

33. An expanded population of cells of claim 26.

34. (canceled)

35. An antibody or an antigen binding fragment thereof or a polypeptide comprising either or both of:
(i) a single chain variable fragment sequence (scFv) comprising an amino acid sequence of (Q V Q L V Q S G V E V K K P G A S V K V S C K A S G Y T F T N Y Y M Y W V R Q A P G Q G L E W M G G I N P S N G G T N F N E K F K N R V T L T T D S S T T T A Y M E L K S L Q F D D T A V Y Y C A R R D Y R F D M G F D Y W G Q G T T V T V S S G G G G S G G G G S G G G G S D I Q M T Q S P S S L S A S V G D R V T I T C R A S Q D V S T A V A W Y Q Q K P G K A P K L L I Y S A S F L Y S G V P S R F S G S G S G T D F T L T I S S L Q P E D F A T Y Y C Q Q Y L Y H P A T F G Q G T K V E I K R (SEQ ID NO: 13) or an equivalent thereof, optionally encoded by the nucleotide sequence comprising a nucleic acid sequence (CAGGTCCAATTGGTACAGAGCGGCGTCGAAGTAAAGAAGCCTGGAGCCAGCGTTA AAGTTTCTTGCAAGGCTTCAGGATATACTTTCACTAACTACTATATGTACTGGGTAC GGCAGGCTCCAGGGCAAGGGTTGGAGTGGATGGGAGGGATCAATCCTTCTAACGGC GGCACTAACTTTAACGAAAAATTTAAAAATAGGGTGACCCTCACAACTGACTCAAG TACGACTACAGCATACATGGAACTCAAATCTCTCCAATTCGATGACACGGCTGTCTA TTATTGCGCGAGAAGAGACTATCGCTTCGATATGGGGTTTGATTATTGGGGGCAAGG TACTACGGTTACCGTCAGCTCCGGGGGTGGCGGCTCCGGCGGCGGTGGGTCAGGTG GAGGAGGGTCTGACATTCAGATGACGCAATCCCCAAGCTCTCTGTCCGCGTCAGTGG GCGACCGAGTTACAATCACATGCCGCGCTTCTCAAGATGTGTCAACCGCTGTCGCCT GGTACCAACAGAAGCCTGGGAAGGCCCCTAAGCTTCTCATCTACTCAGCTTCTTTTC TGTACTCAGGGGTACCGTCTAGATTCTCAGGATCCGGTAGTGGGACGGACTTCACAT TGACCATAAGTTCCTTGCAGCCTGAGGATTTCGCTACATATTATTGCCAACAGTACC TTTACCATCCTGCCACTTTTGGCCAGGGTACTAAGGTCGAGATCAAACGG (SEQ ID NO: 14)), or an equivalent thereof; or
(ii) a scFv comprising an amino acid sequence (EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVT VSAGGGGSGGGGSGGGGSEIVLTQSPATLSLSPGERATLSCRASKGVSTSGYSYLHWYQ QKPGEAPRLLIYLASYLESGVPARFSGSGSGTDFTLTISSLEPEDFAVYYCQHSRDLPLTF GGGTKVEIK (SEQ ID NO: 15)) or an equivalent thereof, optionally encoded by the nucleotide sequence:
(GAAGTTCAGTTGGTCGAGTCAGGAGGAGGCCTGGTGCAACCCGGGGGCT
CACTCCGGTTGTCCTGTGCTGCTTCAGGATTTACGTTTTCTGACTCATGG
ATACATTGGGTGCGCCAAGCCCCGGGCAAGGGGCTGGAATGGGTGGCCTG
GATCTCTCCGTATGGGGGTTCCACCTACTATGCTGATTCAGTAAAAGGAC
GGTTCACTATAAGCGCGGATACAAGTAAGAATACTGCCTATCTTCAAATG
AATTCTCTTCGCGCCGAGGATACAGCGGTATATTATTGCGCTAGACGACA
TTGGCCAGGGGGCTTTGACTATTGGGGGCAGGGTACTCTTGTGACCGTTA
GTGCGGGAGGTGGTGGCAGCGGTGGAGGCGGCTCCGGGGGTGGTGGTTCA
GAAATTGTCCTGACTCAATCCCCTGCCACATTGAGTTTGAGCCCAGGAGA
GAGAGCAACTCTGTCATGCCGGGCGTCAAAAGGTGTCAGTACGTCAGGCT
ACTCCTATCTTCATTGGTATCAGCAGAAACCGGGAGAAGCGCCGCGCCTT
CTCATATACCTGGCTAGTTACCTTGAGAGTGGCGTCCCGGCCCGGTTTAG
TGGGAGTGGGTCTGGGACTGATTTTACGCTGACAATCAGCAGTCTTGAGC
CAGAGGACTTCGCGGTTTACTATTGCCAACATTCACGCGATTTGCCCCTC
ACCTTCGGCGGTGGAACGAAGGTTGAAATAAAA (SEQ ID NO:
16)), or an equivalent thereof.

36.-58. (canceled)

59. A method of producing a CAR expressing cell comprising transducing an isolated cell with the isolated nucleic acid or the vector of claim 1, and wherein the isolated cells are selected from: T-cells, B-cells, NK-cells, dendritic cells, myeloid cells, monocytes, or macrophages.

60. (canceled)

61. A method of inhibiting the growth of a cancer or tumor expressing FLT3, optionally acute myeloid leukemia (AML) in a subject, comprising contacting the cancer or tumor with the isolated cell of claim 26.

62. A method of inhibiting the growth of a cancer or tumor expressing FLT3 in a subject, optionally acute myeloid leukemia (AML), comprising measuring expression of PD-1 or PD-L1 or both PD-1 and PD-L1 in the subject and administering the isolated cell of claim 26 to a subject expressing PD-1 or PD-L1 or both PD-1 and PD-L1.

63. A method of inhibiting the growth of a cancer or tumor in a subject, optionally acute myeloid leukemia (AML), comprising measuring expression of PD-1 or PD-L1 or both PD-1 and PD-L1 in the subject and administering the antibody or antigen binding fragment or polypeptide of claim 35 to a subject expressing PD-1 or PD-L1 or both PD-1 and PD-L1.

64.-70. (canceled)
